(19) United States
(12) Patent Application Publication
(10) Pub. No.: US 2010/0168079 A1
(43) Pub. Date: Jul. 1, 2010

(54) Biaryl Benzylamine Derivatives

(76) Inventors: Daniela Angst, Basel (CH); Birgit Bollbuck, Basel (CH); Philipp Janser, Basel (CH); Jean Quancard, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/643,517

(22) Filed: Dec. 21, 2009

Related U.S. Application Data
(60) Provisional application No. 61/142,668, filed on Jan. 6, 2009.

(30) Foreign Application Priority Data
Dec. 23, 2008 (EP) 08172684.6

(51) Int. Cl. A61K 31/397; C07C 229/40; A61K 31/198; C07D 205/04; C07C 271/22; A61K 31/27; A61P 37/00; A61P 29/00; A61P 17/06; A61P 1/00; A61P 25/28

(52) U.S. Cl. 514/210.17; 562/448; 514/563; 562/449; 562/450; 548/953; 560/27; 514/487

(57) ABSTRACT

The present invention relates to biaryl-benzylamine compounds, to processes for their production, to their use as pharmaceuticals and to pharmaceutical compositions comprising them.

The present invention relates in particular to biaryl-benzylamine compounds, to processes for their production, to their use as pharmaceuticals and to pharmaceutical compositions comprising them.

More particularly, the present invention provides a compound of formula (I) or a salt thereof;

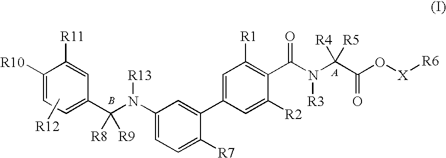

wherein
R1 is C₁-C₆ alkyl, halo, halo C₁-C₆ alkyl;
R2 is H, C₁-C₆ alkyl or halo;
R3 is H, or C₁-C₆ alkyl;
R4 is C₁-C₆ alkyl, optionally substituted by halogen, hydroxyl, C₁-C₆ alkoxy or NR′R″, wherein
R′ and R″ are each independently selected from H, acyl and C₁-C₆ alkyl;
X is a bond or is C₁-C₆ alkylene optionally interrupted by 1-2 O-atoms;
R5 is H or C₁-C₆ alkyl; or
R4 and R5 together with the carbon atom to which they are attached form a 3-6 membered carbocyclic ring which is optionally interrupted by NR15;
R6 is H; C₁-C₆ alkyl, optionally interrupted by 1-2 O-atoms; or C₁-C₆ alkyl substituted by NR16R17;
R7 is H or halo;
R8 is C₁-C₆ alkyl, optionally substituted by halo;
R9 is H, or C₁-C₆ alkyl, optionally substituted by halo;
R10 is C₁-C₆ alkoxy, OH, halo, cyano, or C₁-C₆ alkyl, optionally substituted by halo;
R11 is C₁-C₆ alkoxy, OH, halo, cyano, or C₁-C₆ alkyl, optionally substituted by halo;
R12 is H, C₁-C₆ alkoxy, OH, halo, cyano, or C₁-C₆ alkyl, optionally substituted by halo;
R13 is H or C₁-C₆ alkyl; and R15, R16 and R17 are independently selected from H, acyl and C₁-C₆ alkyl.

In another embodiment the invention provides a compound of formula (Ia) or a salt thereof;

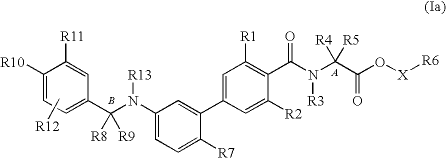

wherein
R1 is C₁-C₆ alkyl;
R2 is H or C₁-C₆ alkyl;
R3 is H, or C₁-C₆ alkyl;
R4 is C₁-C₆ alkyl, optionally substituted by hydroxyl;
R5 is H or C₁-C₆ alkyl; or
R4 and R5 together with the carbon atom to which they are attached form a 3-6 membered carbocyclic ring which is optionally interrupted by NR15;
X is a bond or is C₁-C₆ alkylene, optionally interrupted by 1-2 O-atoms;
R6 is H; C₁-C₆ alkyl, optionally interrupted by 1-2 O-atoms; or C₁-C₆ alkyl substituted by NR16R17;
R7 is H or halo;
R8 is C₁-C₆ alkyl, optionally substituted by halo;
R9 is H, or C₁-C₆ alkyl, optionally substituted by halo;
R10 is halo, or C₁-C₆ alkyl, optionally substituted by halo;
R11 is C₁-C₆ alkyl, optionally substituted by halo;
R12 is H, C₁-C₆ alkoxy, OH, halo, cyano, or C₁-C₆ alkyl, optionally substituted by halo;
R13 is H or C₁-C₆ alkyl; and
R15, R16 and R17 are independently selected from H, acyl and C₁-C₆ alkyl.

In another embodiment the invention provides a compound of formula (Ib) or a salt thereof,

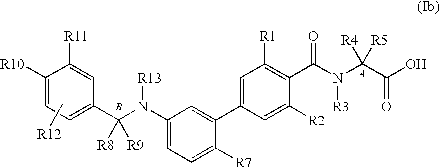

wherein
R1 is C₁-C₆ alkyl;
R2 is H or C₁-C₆ alkyl;

### R3 is H;

R4 is C₁-C₆ alkyl, optionally substituted by hydroxy;
R5 is H or C₁-C₆ alkyl; or
R4 and R5 together with the carbon atom to which they are attached form a 3-6 membered carbocyclic ring;
R7 is H or halo;
R8 is C₁-C₆ alkyl, optionally substituted by halo;
R9 is H, or C₁-C₆ alkyl, optionally substituted by halo;
R10 is halo, or C₁-C₆ alkyl, optionally substituted by halo;
R11 is C₁-C₆ alkyl, optionally substituted by halo;
R12 is H, C₁-C₆ alkoxy, OH, halo, cyano, or C₁-C₆ alkyl, optionally substituted by halo; and

### R13 is H.

In another embodiment the invention provides a compound of formula (Ic) or a salt thereof,

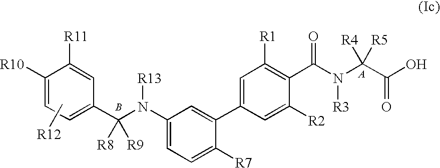

wherein
R1 is C₁-C₆ alkyl;
R2 is C₁-C₆ alkyl;

### R3 is H;

R4 is C₁-C₆ alkyl, optionally substituted by hydroxy;
R5 is H or C₁-C₆ alkyl; or
R4 and R5 together with the carbon atom to which they are attached form a 3-6 membered carbocyclic ring;

### R7 is H;

R8 is C₁-C₆ alkyl, optionally substituted by halo;
R9 is H, or C₁-C₆ alkyl, optionally substituted by halo;
R10 is halo, or C₁-C₆ alkyl, optionally substituted by halo;
R11 is C₁-C₆ alkyl, optionally substituted by halo;
R12 is H or alkyl; and

### R13 is H.

With regard to a compound of formula (I) the following significances represent further embodiments of the invention independently, collectively or in any combination or in any sub-combination thereof:
1. R1 is methyl, chloro or trifluoromethyl;
2. R2 is H; chloro or methyl;
3. R1 and R2 are both methyl;
4. R3 is H;
5. R3 is methyl;
6. R4 is methyl;
7. X is a bond or C₁-C₆ alkylene, and in particular C₁-C₂ alkylene;
8. R5 is H;
9. R4 and R5 together with the carbon atom to which they are attached form a 3-5 membered carbocyclic ring;
10. R6 is H;
11. R7 is H;
12. R7 is fluoro;
13. R8 is methyl, ethyl or trifluoromethyl;
14. R9 is H;
15. R8 and R9 are both methyl;
16. R10 is chloro, fluoro, methyl, or trifluoromethyl;
17. R10 is halo, in particular chloro;
18. R11 is methyl;
19. R12 is H or methyl;
20. R13 is H;
21. R13 is methyl;
22. The stereochemistry of the carbon atom in a compound of formula (I) in position A represents the (S)-configuration provided R5 is hydrogen (carbon atom to which is attached a group R4, and R5 is hydrogen);
23. The stereochemistry of the carbon atom in a compound of formula (I) in position B represents the (R)-configuration provided R9 is hydrogen and R8 represents C₁-C₆ alkyl such as methyl, or ethyl; or the stereochemistry of the carbon atom in a compound of formula (I) in position B represents the (S)-configuration provided R9 is hydrogen and R8 is halo C₁-C₆ alkyl such as trifluoromethyl;
24. R8 is selected from halo C₁-C₆ alkyl, and C₁-C₆ alkyl and R9 is H;
25. R4 is C₁-C₆ alkyl and R5 is hydrogen, or R4 and R5 together with the carbon atom to which they are attached form a 3-5 membered carbocyclic ring;
26. X is a bond and R6 is H.

In another embodiment the invention provides a compound of formula (I) comprising the following combinations of preferences independently, collectively or in any combination or in any sub-combination thereof as provided in the above section:
i. Item 3, 4, 8 and 11, and the other variables are as defined for formula (I),
ii. Item 3, 4, 8 and 12, and the other variables are as defined for formula (I),
iii. Item 3, 4, 6, 8, 10 and 11, and the other variables are as defined for formula (I),
iv. Item 3, 4, 6, 8, 10 and 12, and the other variables are as defined for formula (I),
v. Item 3, 4, 9, and 10, and the other variables are as defined for formula (I),
vi. Item 3, 9, and 10, and the other variables are as defined for formula (I),
vii. Item 3, 6, 8, 10 and 12, and the other variables are as defined for formula (I),
viii. Item 3, 4, 6, 8, 10 and 11, and the other variables are as defined for formula (I),
ix. Item 1, 2, 4, 6, 8, 10 and 17, and the other variables are as defined for formula (I),
x. Item 1, 2, 9, 10 and 11, and the other variables are as defined for formula (I),
xi. Item 1, 2, 4, 8 and 11, and the other variables are as defined for formula (I),
xii. Item 1, 2, 4, 8 and 12, and the other variables are as defined for formula (I),
xiii. Item 1, 2, 4, 6, 8, 10 and 11, and the other variables are as defined for formula (I),
xiv. Item 1, 2, 4, 6, 8, 10 and 12, and the other variables are as defined for formula (I),
xv. Item 1, 2, 4, 9, and 10, and the other variables are as defined for formula (I),
xvi. Item 1, 2, 9, and 10, and the other variables are as defined for formula (I),
xvii. Item 1, 2, 6, 8, 10 and 12, and the other variables are as defined for formula (I),
xviii. Item 1, 2, 4, 6, 8, 10 and 11, and the other variables are as defined for formula (I),
xix. Item 1, 2, 4, 6, 8, 10 and 17, and the other variables are as defined for formula (I),
xx. Item 1, 2, 9, 10 and 11, and the other variables are as defined for formula (I).

The compounds of the invention may exist in free form or in salt form, in particular in pharmaceutically acceptable salt form, e.g. addition salts with e.g. organic or inorganic acids, for example, hydrochloric acid or acetic acid, or salts obtainable when a compound of formula (I) comprises COON, with a base, e.g. alkali salts such as sodium or potassium, or unsubstituted or substituted ammonium salts, e.g. N-methyl-D-glucamine or D-glucamine.

Compounds of the present invention in particular compounds of formula (I) comprising a pharmaceutically acceptable salt are provided in another aspect.

By a pharmaceutically-acceptable and -cleavable ester or a physiologically hydrolysable derivative of a compound of formula (I) is meant a compound which is hydrolysable under physiological conditions to yield a compound of formula (I) and a by-product which is itself physiologically acceptable, e.g. an ester which is hydrolyzed to yield a compound of formula (I) and a non-toxic alcohol at the desired dosage levels. The compounds of formula (I) may represent such pharmaceutically-acceptable and -cleavable esters or physiologically hydrolysable derivatives. For example an ester of formula (I), e.g. R6=alkyl, may be typically converted into an acid of formula (I), e.g. R6=H. The present invention typically encompasses both the uncleaved and/or cleaved as well the unhydrolysed and/or hydrolysed compounds/derivatives.

For the avoidance of doubt, the terms listed below are to be understood to have the following meaning throughout the present description and claims:

The term “lower”, when referring to organic radicals or compounds means a compound or radical which may be branched or unbranched with up to and including 7 carbon atoms.

An alkyl may be branched, unbranched or cyclic. C₁-C₆ alkyl represents, for example: methyl, ethyl, propyl, butyl, isopropyl, isobutyl, tertiary butyl or 2,2-dimethylpropyl. In accordance to the foregoing, a cycloalkyl represents a cyclic hydrocarbon containing from 3 to 12 ring atoms preferably from 3 to 6 ring atoms. Cycloalkyl represents, for example: cyclopropyl, cyclobutyl, cyclopentyl, or cyclohexyl. The cycloalkyl may optionally be substituted.

An alkoxy group may be branched or unbranched. C₁-C₆ alkoxy represents, for example: methoxy, ethoxy, propoxy, butoxy, isopropoxy, isobutoxy or tertiary butoxy. Alkoxy includes cycloalkyloxy and cycloalkyl-alkyloxy.

An alkene, alkenyl or alkenoxy group is branched or unbranched and contains 2 to 10 carbon atoms, preferably 2 to 4 carbon atoms and contains at least one carbon-carbon double bond. Alkene, alkenyl or alkenoxy represents for example vinyl, prop-1-enyl, allyl, butenyl, isopropenyl or isobutenyl and the oxy equivalents thereof.

An alkyne or alkynyl group is branched or unbranched and contains 2 to 10 carbon atoms, preferably 1 to 4 carbon atoms and contains at least one carbon-carbon triple bond. Lower alkyne or lower alkynyl or lower alkenyloxy represents for example ethynyl or propynyl.

In the present application, oxygen containing substituents, e.g. alkoxy, alkenyloxy, alkynyloxy, carbonyl, etc. encompass their sulphur containing homologues, e.g. thioalkyl, alkyl-thioalkyl, thioalkenyl, alkenyl-thioalkyl, thioalkynyl, thiocarbonyl, sulphone, sulphoxide etc.

Halo or halogen represents chloro, fluoro, bromo or iodo. Preferably halo or halogen represents chloro or fluoro.

Haloalkyl refers to an alkyl as defined herein, that is substituted by one or more halo groups as defined herein. Preferably the haloalkyl can be monohaloalkyl, dihaloalkyl or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Preferably, the polyhaloalkyl contains up to 12, or 10, or 8, or 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

As used herein acyl is a radical R_{d}CO wherein R_{d} is H, C₃₋₆cycloalkyl, C₃₋₆cycloalkyloxy, C₁₋₆alkoxy, phenyl, phenyloxy, benzyl or benzyloxy, preferably acyl is C₁₋₆alkyl-CO, C₁₋₆alkoxy-CO, benzyloxy-CO or benzyl-CO, more preferably C₁₋₆alkyl-CO or C₁₋₄alkoxy-CO, particularly C₁₋₄alkyl-CO, C₁₋₄alkoxy-CO, t-butoxycarbonyl or acetyl (CH₃CO).

Aryl represents carbocyclic aryl or biaryl.

Carbocyclic aryl is an aromatic cyclic hydrocarbon containing from 6 to 18 ring atoms. It can be monocyclic, bicyclic or tricyclic, for example naphthyl, phenyl, or phenyl mono-, di- or trisubstituted by one, two or three substituents.

Heterocyclic aryl or heteroaryl is an aromatic monocyclic or bicyclic hydrocarbon containing from 5 to 18 ring atoms one or more of which are heteroatoms selected from O, N or S. Preferably there are one to three heteroatoms. Heterocyclic aryl represents, for example: pyridyl, indolyl, quinoxalinyl, quinolinyl, isoquinolinyl, benzothienyl, benzofuranyl, benzothiophenyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, oxadiazolyl, benzimidazolyl, benzthiazolyl, benzoxazolyl, Heterocyclic aryl also includes such substituted radicals.

Heterocycloalkyl represents a mono-, di- or tricyclic hydrocarbon which may be saturated or unsaturated and which contains one or more, preferably one to three heteroatoms selected from O, N or S. Preferably it contains between three and 18 ring atoms, more preferably between 3 and 8 ring atoms. Heterocycloalkyl represents for example morpholinyl, piperazinyl, piperidinyl, imidazolidinyl, pyrrolidinyl, pyrazolidinyl.

A 3-6 membered carbocyclic ring interrupted by nitrogen or a NR15 group is azacycloalkyl and may be substituted one or more times by C₁-C₆ alkyl such as methyl, ethyl, propyl and the like, said C₁-C₆ alkyl may be attached to a carbon and/or to a nitrogen atom of said azacycloalkyl, and said azacycloalkyl may be attached to the remaining portion of the molecule of formula (I) as defined above. Examples of 3-6 membered azacycloalkyl include piperazinyl, piperidinyl, imidazolidinyl, pyrrolidinyl and azetidinyl.

As used herein, the term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, sodium, potassium, ammonium, calcium, magnesium, iron, silver, zinc, copper, and the like; particularly preferred are the ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like, specifically such as, isopropylamine, benzathine, cholinate, diethanolamine, diethylamine, lysine, piperazine, procaine, N-methyl-D-glucamine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts Properties, Selection, and Use” by Stahl and Wermuth (Wiley-VCH, Weinheim, Germany, 2002).

The present invention includes all pharmaceutically acceptable isotopically-labeled compounds of the invention, i.e. compounds of formula (I), wherein (1) one or more atoms are replaced by atoms having the same atomic number, but an atomic mass or mass number different from the atomic mass or mass number usually found in nature, and/or (2) the isotopic ratio of one or more atoms is different from the naturally occurring ratio.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁸I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labeled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by the S1P receptor, or (ii) associated with the S1P receptor activity, or (ii) characterized by abnormal activity of the S1P receptor; or (2) reducing or inhibiting the activity of the S1P receptor or (3) reducing or inhibiting the expression of the S1P receptor. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of the SIP receptor; or at least partially reducing or inhibiting the expression of the SIP receptor. The meaning of the term “a therapeutically effective amount” as illustrated in the above embodiment for the SIP receptor also applies by the same means to any other relevant proteins/peptides/enzymes.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “inhibition” or “inhibiting” refers to the reduction or suppression of a given condition, symptom, or disorder, or disease, or a significant decrease in the baseline activity of a biological activity or process.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (ag. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Compounds of the present invention are either obtained in the free form, as a salt thereof, or as prodrug derivatives thereof.

When both a basic group and an acid group are present in the same molecule, the compounds of the present invention may also form internal salts, e.g., zwitterionic molecules.

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, that contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers etc.

Typically, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with carrier. Carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

In a further aspect the invention provides a process for preparing a compound of formula (I) in free or salt form, comprising:
a) For compounds of formula (I) wherein R9 and R13 are H, the step of reductive amination between an aniline of formula (II) and a ketone of formula (III) using standard reducing agents, e.g. decaborane, sodium cyanoborohydride or sodium triacetoxyborohydride, followed by an optional deprotection step:

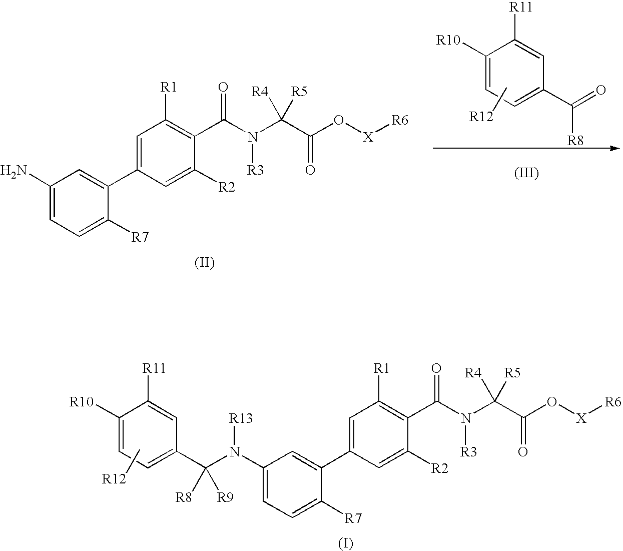

b) For compounds of formula (I) wherein R9 is H, the step of in situ double reductive amination between an aniline of formula (II) and a ketone of formula (III) followed by an aldehyde of formula (IV), wherein R′″ is H or C₁-C₅ alkyl, using standard reducing agents, e.g. decaborane, sodium cyanoborohydride or sodium triacetoxyborohydride, followed by an optional deprotection step:

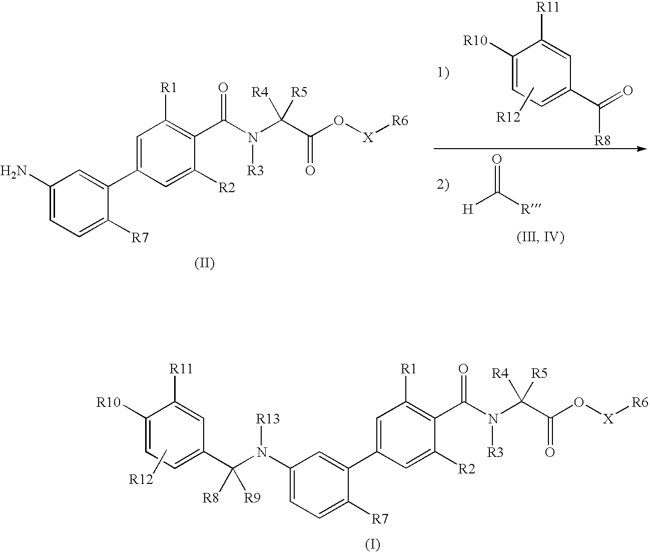

c) For compounds of formula (I) the step of coupling a carboxylic acid of formula (V) with an optionally protected amine of formula (VI) or a salt thereof using standard coupling reagents, e.g. TBTU or HATU, and a base, e.g. Hünig's base or triethylamine, followed by an optional deprotection step:

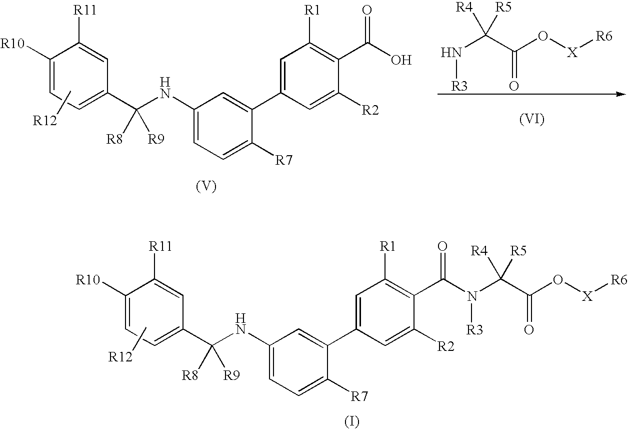

d) For compounds of formula (I) the step of palladium-catalyzed Suzuki coupling of a boronic acid derivative of formula (VIII) with a halide of formula (VII) or a salt thereof using standard palladium catalysts, e.g. Pd(PPh₃)₄ or PdCl₂(PPh₃)₂ or Pd(OAc)₂ with 2-dicyclohexylphosphino-2′,6′-dimethoxybiphenyl, and a base, e.g. sodium bicarbonate or potassium phosphate, followed by an optional deprotection step:

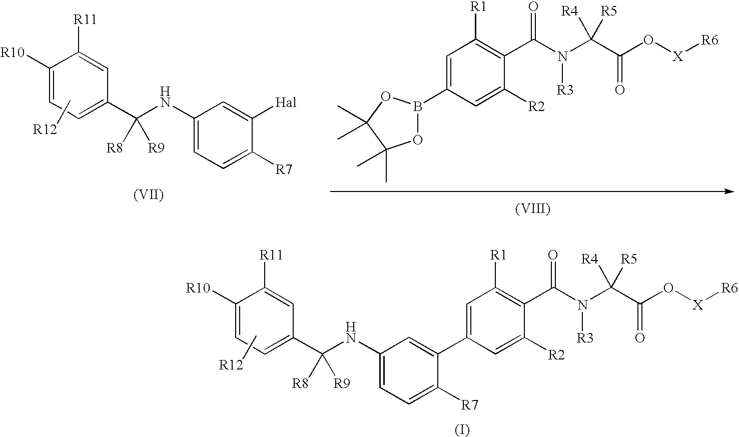

In a further aspect the invention provides a process for preparing a compound of formula (II) in free or salt form, comprising:
a) For compounds of formula (II) the step of palladium-catalyzed Suzuki coupling of a boronic acid derivative of formula (IX) with a bromide of formula (X) or a salt thereof using standard palladium catalysts, e.g. Pd(PPh₃)₄ or PdCl₂(PPh₃)₂ or Pd(OAc)₂ with 2-dicyclohexylphosphino-2′,6′-dimethoxybiphenyl, and a base, e.g. sodium bicarbonate or potassium phosphate:

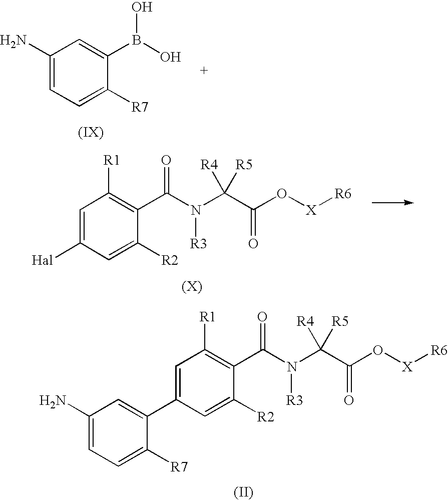

In a further aspect the invention provides a process for preparing a compound of formula (V) in free or salt form, comprising:
a) For compounds of formula (V) the step of palladium-catalyzed Suzuki coupling of a boronic acid derivative of formula (XII) with a halide of formula (XI) or a salt thereof using standard palladium catalysts, e.g. Pd(PPh₃)₄ or PdCl₂(PPh₃)₂ or Pd(OAc)₂ and 2-dicyclohexylphosphino-2′,6′-dimethoxybiphenyl, and a base, e.g. sodium bicarbonate or potassium phosphate:

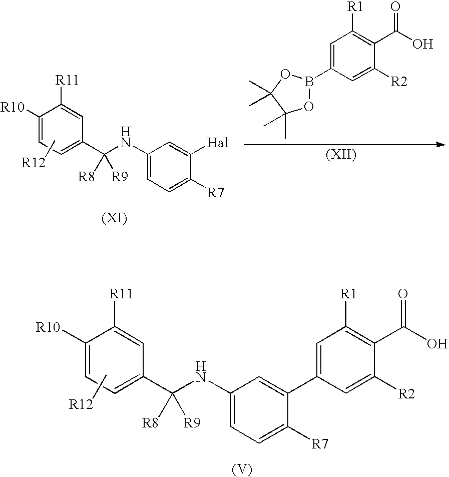

In a further aspect the invention provides a process for preparing a compound of formula (VII) in free or salt form, comprising:
a) For compounds of formula (VII) the step of palladium-catalyzed Buchwald-Hartwig coupling of an amine of formula (XIII) or a salt thereof with a di-halide of formula (XIV) using standard palladium catalysts, e.g. PEPPSI-IPr®, and a base, e.g. potassium t-butoxide:

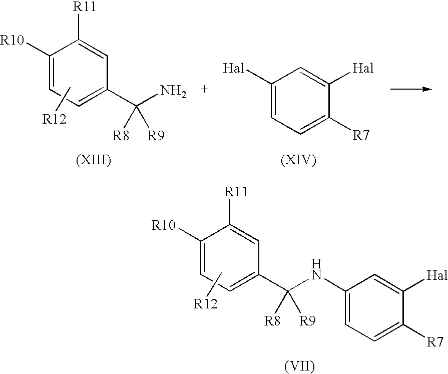

In a further aspect, the invention provides a process for preparing the compounds of the invention by a reaction sequence involving (i) the reaction of a ketone with an aniline (as shown in the below scheme) with a reductive step furnishing a racemic intermediate optionally followed by a chiral separation to furnish the chiral intermediate, or (ii) alternatively by an enantioselective step in accordance to Dong Pei et al. Organic Letters, 2006, 5913-5915, furnishing a chiral intermediate, followed by (iii) a Suzuki-type coupling with an appropriate boronic acid or ester, followed by (iv) an amide coupling with an appropriate amino ester, and optionally followed by (v) deprotection step to the carboxylic acid derivatives as shown in the below Scheme, wherein the reaction conditions are typically in accordance to those provided hereinabove or in the experimental section and wherein the variables have the definitions provided hereinabove, in particular as defined for formula (I):

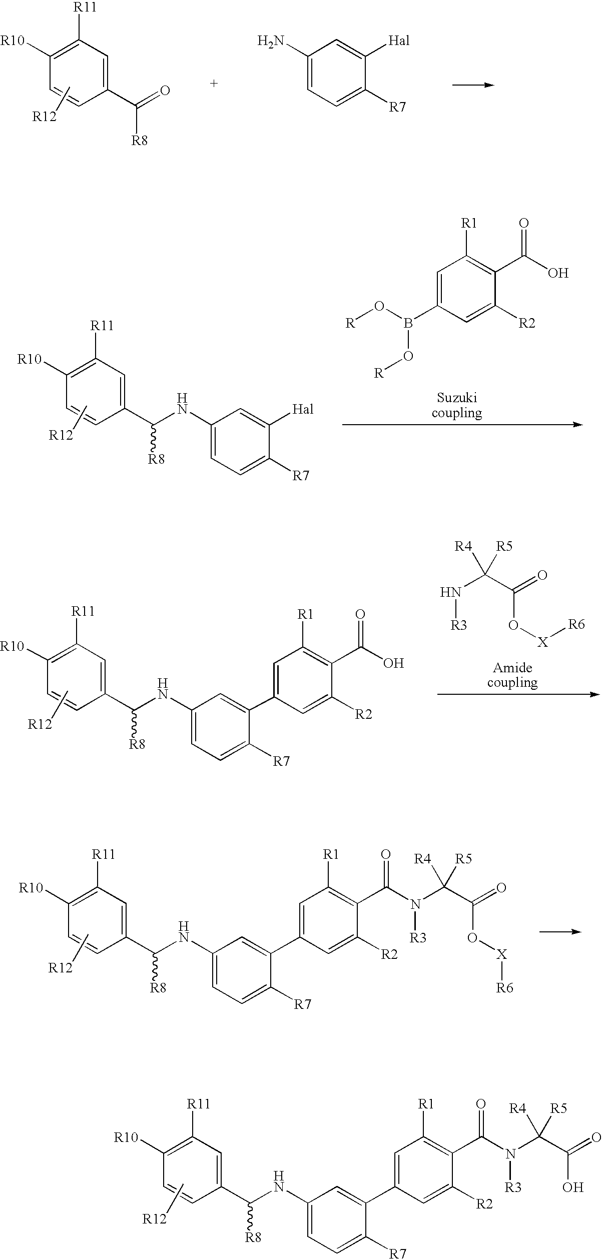

As used in the above process descriptions, an optional deprotection step means typically the hydrolysis of an ester under basic conditions, using e.g. LiOH or NaOH or KOH in a mixture of water and an organic solvent, e.g. THF or EtOH, or the cleavage of acid labile groups e.g. tertiary-butyl esters or BOC-protected amines, under acidic conditions, using e.g. TFA or HCl in an aprotic organic solvent e.g. dichloromethane or diethylether.

The compounds of the invention can be recovered from the reaction mixture and purified in conventional manner. Isomers, such as enantiomers, may be obtained in conventional manner, e.g. by fractional crystallization typically using chiral auxiliaries or optionally by separation involving chiral phases or by asymmetric synthesis from corresponding asymmetrically substituted, e.g. optically active starting materials.

Preferred compounds of formula (I) are:
(S)-2-({3′-[1-(3-Chloro-4-methoxy-phenyl)-ethylamino]-3,5-dimethyl-biphenyl-4-carbonyl}-amino)-propionic acid
(S)-2-({3′-[(4-Chloro-3-methyl-phenyl)-ethylamino]-3,5-dimethyl-biphenyl-4-carbonyl}-amino)-propionic acid
(S)-2-({3′-[(R)-1-(4-Chloro-3-methyl-phenyl)-ethylamino]-3,5-dimethyl-biphenyl-4-carbonyl}-amino)-propionic acid
(S)-2-({3′-[(S)-1-(4-Chloro-3-methyl-phenyl)-ethylamino]-3,5-dimethyl-biphenyl-4-carbonyl}-amino)-propionic acid
(S)-2-({3′-[1-(4-Chloro-3-methyl-phenyl)-propylamino]-3,5-dimethyl-biphenyl-4-carbonyl}-amino)-propionic acid
(S)-2-({3′-[(R)-1-(4-Chloro-3-methyl-phenyl)-propylamino]-3,5-dimethyl-biphenyl-4-carbonyl}-amino)-propionic acid
1-({3′-[1-(4-Chloro-3-methyl-phenyl)-ethylamino]-3,5-dimethyl-biphenyl-4-carbonyl}-amino)-cyclopropanecarboxylic acid
1-({3′-[(1-(4-Chloro-3-methyl-phenyl)-ethylamino]-3,5-dimethyl-biphenyl-4-carbonyl}-amino)-cyclopropanecarboxylic acid
(S)-2-({3′-[1-(4-Chloro-3-fluoro-phenyl)-ethylamino]-3,5-dimethyl-biphenyl-4-carbonyl}-amino)-propionic acid
(S)-2-({3′-[1-(3,4-Dichlorophenyl)-ethylamino]-3,5-dimethyl-biphenyl-4-carbonyl}-amino)-propionic acid
(S)-2-({3′-[1-(3,4-Dimethylphenyl)-ethylamino]-3,5-dimethyl-biphenyl-4-carbonyl}-amino)-propionic acid
(R)-2-({3′-[1-(4-Chloro-3-methyl-phenyl)-ethylamino]-3,5-dimethyl-biphenyl-4-carbonyl}-amino)-propionic acid
(S)-2-({3′-[1-(4-Chloro-2,5-dimethyl-phenyl)-ethylamino]-3,5-dimethyl-biphenyl-4-carbonyl}-amino)-propionic acid
(S)-2-({3′-[1-(4-Chloro-3-trifluoromethyl-phenyl)-ethylamino]-3,5-dimethyl-biphenyl-4-carbonyl}-methyl-amino)-propionic acid
(S)-2-({3′-[1-(4-Fluoro-3-methoxy-phenyl)-ethylamino]-3,5-dimethyl-biphenyl-4-carbonyl}-amino)-propionic acid
(S)-2-({3′-[1-(4-Chloro-2-fluoro-5-methyl-phenyl)-ethylamino]-3,5-dimethyl-biphenyl-4-carbonyl}-amino)-propionic acid
(S)-2-({3′-[1-(4-Fluoro-3-trifluoromethoxy-phenyl)-ethylamino]-3,5-dimethyl-biphenyl-4-carbonyl}-amino)-propionic acid
(S)-2-({3′-[1-(3-Fluoro-4-methoxy-phenyl)-ethylamino]-3,5-dimethyl-biphenyl-4-carbonyl}-amino)-propionic acid
(S)-2-({3′-[1-(3-Fluoro-5-methyl-phenyl)-ethylamino]-3,5-dimethyl-biphenyl-4-carbonyl}-amino)-propionic acid
(S)-2-({3′-[1-(3,4-Dichloro-phenyl)-propylamino]-3,5-dimethyl-biphenyl-4-carbonyl}-amino)-propionic acid
(S)-2-({3-[1-(4-Chloro-3-methyl-phenyl)-ethylamino]-3,5-dimethyl-biphenyl-4-carbonyl}-amino)-3-hydroxy-propionic acid
(S)-2-({5′-[1-(4-Chloro-3-methyl-phenyl)-ethylamino]-2′-fluoro-3,5-dimethyl-biphenyl-4-carbonyl}-amino)-propionic acid
(S)-2-({5′-[(R)-1-(4-Chloro-3-methyl-phenyl)-ethylamino]-2′-fluoro-3,5-dimethyl-biphenyl-4-carbonyl}-amino)-propionic acid
(S)-2-({5′-[1-(4-Chloro-3-methyl-phenyl)-propylamino]-2′-fluoro-3,5-dimethyl-biphenyl-4-carbonyl}-amino)-propionic acid
1-({5′-[1-(4-Chloro-3-methyl-phenyl)-propylamino]-2′-fluoro-3,5-dimethyl-biphenyl-4-carbonyl}-amino)-cyclopropanecarboxylic acid
1-({3′-[1-(4-Chloro-3-methyl-phenyl)-ethylamino]-2′-fluoro-3,5-dimethyl-biphenyl-4-carbonyl}-amino)-cyclopropanecarboxylic acid
1-({3′-[1-(4-Chloro-3-methyl-phenyl)-propylamino]-3,5-dimethyl-biphenyl-4-carbonyl}-amino)-cyclopropanecarboxylic acid
(S)-2-({3′-[1-(3,5-Dimethyl-phenyl)-ethylamino]-3,5-dimethyl-biphenyl-4-carbonyl}-amino)-propionic acid
(S)-2-({3′-[1-(4-Chloro-3,5-dimethyl-phenyl)-ethylamino]-3,5-dimethyl-biphenyl-4-carbonyl}-amino)-propionic acid
(S)-2-({3′-[1-(4-Methoxy-3-methyl-phenyl)-ethylamino]-3,5-dimethyl-biphenyl-4-carbonyl}-amino)-propionic acid
(S)-2-({3′-[1-(4-Methoxy-phenyl)-ethylamino]-3,5-dimethyl-biphenyl-4-carbonyl}-amino)-propionic acid
(S)-2-({3′-[1-(3,4-Dimethoxy-phenyl)-ethylamino]-3,5-dimethyl-biphenyl-4-carbonyl}-amino)-propionic acid
(S)-2-({3′-[1-(4-Methoxy-3-trifluoromethyl-phenyl)-ethylamino]-3,5-dimethyl-biphenyl-4-carbonyl}-amino)-propionic acid
(S)-2-({3′-[1-(3,5-Difluoro-4-methoxy-phenyl)-ethylamino]-3,5-dimethyl-biphenyl-4-carbonyl}-amino)-propionic acid
(S)-2-({3′-[1-(2-Fluoro-4-methoxy-phenyl)-ethylamino]-3,5-dimethyl-biphenyl-4-carbonyl}-amino)-propionic acid
(S)-2-({3′-[(3-Chloro-5-fluoro-4-methoxy-phenyl)-ethylamino]-3,5-dimethyl-biphenyl-4-carbonyl}-amino)-propionic acid
(S)-2-({3′-[(4-Methoxy-3,5-dimethyl-phenyl)-ethylamino]-3,5-dimethyl-biphenyl-4-carbonyl}-amino)-propionic acid
(S)-2-({3′-[1-(4-Chloro-3-methyl-phenyl)-ethylamino]-3-methyl-biphenyl-4-carbonyl}-amino)-propionic acid
(S)-2-({3′-[(S)-1-(4-Chloro-3-methyl-phenyl)-ethylamino]-3-methyl-biphenyl-4-carbonyl}-amino)-propionic acid
(S)-2-({3′-[(R)-1-(4-Chloro-3-methyl-phenyl)-ethylamino]-3-methyl-biphenyl-4-carbonyl}-amino)-propionic acid
(S)-2-({3′-[(4-Chloro-2,5-dimethyl-phenyl)-ethylamino]-3-methyl-biphenyl-4-carbonyl}-amino)-propionic acid
(S)-2-({3′-[1-(4-Chloro-3-methyl-phenyl)-ethylamino]-3-ethyl-biphenyl-4-carbonyl}-amino)-propionic acid
(S)-2-({3′-[1-(4-Chloro-3-methyl-phenyl)-ethylamino]-3-ethyl-biphenyl-4-carbonyl}-methyl-amino)-propionic acid
(S)-2-({3′-[1-(4-Chloro-3,5-dimethyl-phenyl)-ethylamino]-3-ethyl-biphenyl-4-carbonyl}-amino)-propionic acid
(S)-2-({3′-[1-(4-Chloro-3,5-dimethyl-phenyl)-ethylamino]-3-ethyl-biphenyl-4-carbonyl}-methyl-amino)-propionic acid
(S)-2-({3-Chloro-3′-[1-(4-chloro-3-methyl-phenyl)-ethylamino]-biphenyl-4-carbonyl}-amino)-propionic acid
(S)-2-({3-Chloro-3′-[1-(4-chloro-3-methyl-phenyl)-ethylamino]-biphenyl-4-carbonyl}-methyl-amino)-propionic acid
(S)-2-({3′-[1-(4-Chloro-3-methyl-phenyl)-ethylamino]-3-fluoro-biphenyl-4-carbonyl}-amino)-propionic acid
(S)-2-({3′-[(4-Chloro-3-methyl-phenyl)-ethylamino]-3-fluoro-biphenyl-4-carbonyl}-methyl-amino)-propionic acid
(S)-2-({3′-[1-(4-Chloro-3-methyl-phenyl)-ethylamino]-3,5-difluoro-biphenyl-4-carbonyl}-amino)-propionic acid
(S)-2-({3′-[1-(4-Chloro-3-methyl-phenyl)-ethylamino]-3,5-difluoro-biphenyl-4-carbonyl}-methyl-amino)-propionic acid
(S)-2-({3′-Chloro-3′-[1-(4-chloro-3,5-dimethyl-phenyl)-ethylamino]-biphenyl-4-carbonyl}-amino)-propionic acid
(S)-2-({3-Chloro-3′-[1-(4-chloro-3,5-dimethyl-phenyl)-ethylamino]-biphenyl-4-carbonyl}-methyl-amino)-propionic acid
(S)-2-({3′-[(4-Chloro-3,5-dimethyl-phenyl)-ethylamino]-3-fluoro-biphenyl-4-carbonyl}-amino)-propionic acid
(S)-2-({3′-[1-(4-Chloro-3,5-dimethyl-phenyl)-ethylamino]-3-fluoro-biphenyl-4-carbonyl}-methyl-amino)-propionic acid
(S)-2-({3′-[1-(4-Chloro-3,5-dimethyl-phenyl)-ethylamino]-3,5-difluoro-biphenyl-4-carbonyl}-amino)-propionic acid
(S)-2-({3′-[(4-Chloro-3,5-dimethyl-phenyl)-ethylamino]-3,5-difluoro-biphenyl-4-carbonyl}-methyl-amino)-propionic acid
(S)-2-({3′-[(4-Chloro-3-methyl-phenyl)-propylamino]-3-methyl-biphenyl-4-carbonyl}-methyl-amino)-propionic acid
(S)-2-({3′-[1-(4-Chloro-3,5-dimethyl-phenyl)-ethylamino]-3-methyl-biphenyl-4-carbonyl}-methyl-amino)-propionic acid
(S)-2-({3′-[1-(4-chloro-3-methyl-phenyl)-propylamino]-biphenyl-4-carbonyl}-amino)-propionic acid
(S)-2-({3′-[1-(4-Chloro-3′-3-methyl-phenyl)-propylamino]-biphenyl-4-carbonyl}-methyl-amino)-propionic acid
(S)-2-({3′-[1-(4-Chloro-3-methyl-phenyl)-ethylamino]-3-trifluoromethyl-biphenyl-4-carbonyl}-amino)-propionic acid
(S)-2-({3′-[1-(4-Chloro-3-methyl-phenyl)-ethylamino]-3-trifluoromethyl-biphenyl-4-carbonyl}-methyl-amino)-propionic acid
(S)-2-({3′-[1-(4-Chloro-3,5-dimethyl-phenyl)-ethylamino]-3-trifluoromethyl-biphenyl-4-carbonyl}-amino)-propionic acid
(S)-2-({3′-[1-(4-Chloro-3,5-dimethyl-phenyl)-ethylamino]-3-trifluoromethyl-biphenyl-4-carbonyl}-methyl-amino)-propionic acid
(S)-2-({3′-[(4-Chloro-3-methyl-phenyl)-propylamino]-3-trifluoromethyl-biphenyl-4-carbonyl}-amino)-propionic acid
(S)-2-({3′-[1-(4-Chloro-3-methyl-phenyl)-propylamino]-3-trifluoromethyl-biphenyl-4-carbonyl}-methyl-amino)-propionic acid
({3′-[1-(4-Chloro-3-methyl-phenyl)-ethylamino]-3,5-dimethyl-biphenyl-4-carbonyl}-amino)-acetic acid
(S)-2-({3′-[1-(4-Chloro-3-methyl-phenyl)-ethylamino]-3,5-dimethyl-biphenyl-4-carbonyl}-amino)-3-methoxy-propionic acid
(S)-6-Amino-2-({3′-[1-(4-chloro-3-methyl-phenyl)-ethylamino]-3,5-dimethyl-biphenyl-4-carbonyl}-amino)-hexanoic acid
2-({3′-[1-(4-Chloro-3-methyl-phenyl)-ethylamino]-3,5-dimethyl-biphenyl-4-carbonyl}-amino)-2-methyl-propionic acid
1-({3′-[1-(4-Chloro-3-methyl-phenyl)-ethylamino]-3,5-dimethyl-biphenyl-4-carbonyl}-amino)-cyclobutanecarboxylic acid
(S)-2-({3′-[(R)-1-(4-Chloro-3-methyl-phenyl)-ethylamino]-3,5-dimethyl-biphenyl-4-carbonyl}-methyl-amino)-propionic acid
(S)-2-({3′-[(4-Chloro-3-methyl-phenyl)-2,2,2-trifluoro-ethylamino]-3,5-dimethyl-biphenyl-4-carbonyl}-amino)-propionic acid
(S)-2-({3′-[(4-Chloro-3-methyl-phenyl)-2,2-difluoro-ethylamino]-3,5-dimethyl-biphenyl-4-carbonyl}-amino)-propionic acid
(S)-2-({3′-[1-(4-Chloro-3-methyl-phenyl)-ethylamino]-3,5-dimethyl-biphenyl-4-carbonyl}-methyl-amino)-propionic acid
(S)-2-({3′-[1-(4-Chloro-phenyl)-ethylamino]-3-methyl-biphenyl-4-carbonyl}-methyl-amino)-propionic acid
(S)-2-({3′-[1-(3,4-Dichloro-phenyl)-ethylamino]-3-methyl-biphenyl-4-carbonyl}-methyl-amino)-propionic acid
3-({3′-[1-(4-Chloro-3-methyl-phenyl)-ethylamino]-3,5-dimethyl-biphenyl-4-carbonyl}-amino)-azetidine-3-carboxylic acid
(S)-2-(3′-{[1-(4-Chloro-3-methyl-phenyl)-ethyl]-methyl-amino}-3,5-dimethyl-biphenyl-4-carbonyl)-amino]-propionic acid
1-({3′-[(R)-1-(4-Chloro-3-methyl-phenyl)-ethylamino]-3,5-dimethyl-biphenyl-4-carbonyl}-methyl-amino)-cyclopropanecarboxylic acid
(S)-2-({3′-[1-(4-Chloro-3-methyl-phenyl)-1-methyl-ethylamino]-3,5-dimethyl-biphenyl-4-carbonyl}-amino)-propionic acid
(S)-2-({3′-[1-(4-Chloro-3-methyl-phenyl)-1-methyl-ethylamino]-3,5-dimethyl-biphenyl-4-carbonyl}-methyl-amino)-propionic acid
1-({3′-[1-(4-Chloro-3-methyl-phenyl)-1-methyl-ethylamino]-3,5-dimethyl-biphenyl-4-carbonyl}-amino)-cyclopropanecarboxylic acid
1-({3′-[1-(4-Chloro-3-methyl-phenyl)-1-methyl-ethylamino]-3,5-dimethyl-biphenyl-4-carbonyl}-methyl-amino)-cyclopropanecarboxylic acid
(S)-2-({5′-[1-(4-Chloro-3-methyl-phenyl)-propylamino]-2′-fluoro-3-methyl-biphenyl-4-carbonyl}-methyl-amino)-propionic acid
(S)-2-({3′-[1-(4-chloro-3-methyl-phenyl)-ethylamino]-2′-fluoro-biphenyl-4-carbonyl}-methyl-amino)-propionic acid
(S)-2-({3′Chloro-5′-[1-(4-chloro-3-methyl-phenyl)-propylamino]-2′-fluoro-biphenyl-4-carbonyl}-methyl-amino)-propionic acid
(S)-2-({5-[1-(4-Chloro-3-methyl-phenyl)-propylamino]-2′-fluoro-3-trifluoromethyl-biphenyl-4-carbonyl}-methyl-amino)-propionic acid
(S)-2-({3′-[1-(4-Chloro-3-methyl-phenyl)-2-methyl-propylamino]-3,5-dimethyl-biphenyl-4-carbonyl}-amino)-propionic acid
(S)-2-({3′-[(4-Chloro-3-methyl-phenyl)-2,2,2-trifluoro-ethylamino]-3-trifluoromethyl-biphenyl-4-carbonyl}-methyl-amino)-propionic acid
(S)-2-({3′-[(4-Chloro-3-methyl-phenyl)-2,2,2-trifluoro-ethylamino]-3-trifluoromethyl-biphenyl-4-carbonyl}-amino)-propionic acid
1-({3-[1-(4-Chloro-3-methyl-phenyl)-2,2,2-trifluoro-ethylamino]-3-trifluoromethyl-biphenyl-4-carbonyl}-methyl-amino)-cyclopropanecarboxylic acid
(S)-2-({3-Chloro-3′-[1-(4-chloro-3-methyl-phenyl)-2,2,2-trifluoro-ethylamino]-biphenyl-4-carbonyl}-amino)-propionic acid
1-({3-Chloro-3′-[1-(4-chloro-3-methyl-phenyl)-2,2,2-trifluoro-ethylamino]-biphenyl-4-carbonyl}-amino)-cyclopropanecarboxylic acid
(S)-2-({3-Chloro-3′-[(S)-1-(4-chloro-3-methyl-phenyl)-2,2,2-trifluoro-ethylamino]-biphenyl-4-carbonyl}-methyl-amino)-propionic acid
(S)-2-({3-Chloro-3′-[1-(4-chloro-3-methyl-phenyl)-1-methyl-ethylamino]-biphenyl-4-carbonyl}-amino)-propionic acid
(S)-2-({3-Chloro-3′-[1-(4-chloro-3-methyl-phenyl)-1-methyl-ethylamino]-biphenyl-4-carbonyl}-methyl-amino)-propionic acid
1-({3′-[1-(4-Chloro-3,5-dimethyl-phenyl)-ethylamino]-3,5-dimethyl-biphenyl-4-carbonyl}-amino)-cyclopropanecarboxylic acid
(S)-2-({3′-[(R)-1-(4-Chloro-3,5-dimethyl-phenyl)-ethylamino]-3,5-dimethyl-biphenyl-4-carbonyl}-amino)-propionic acid
(S)-2-({3′-[(R)-1-(4-Chloro-3,5-dimethyl-phenyl)-propylamino]-3,5-dimethyl-biphenyl-4-carbonyl}-amino)-propionic acid
1-({3′-[1-(4-Chloro-3,5-dimethyl-phenyl)-propylamino]-3,5-dimethyl-biphenyl-4-carbonyl}-amino)-cyclopropanecarboxylic acid
(S)-2-({3′-[1-(4-Chloro-3-methyl-phenyl)-2,2-difluoro-ethylamino]-3,5-dimethyl-biphenyl-4-carbonyl}-methyl-amino)-propionic acid
(S)-2-[(3′-{[(R)-1-(4-Chloro-3-methyl-phenyl)-propyl]-methyl-amino]-3,5-dimethyl-biphenyl-4-carbonyl}-amino)-propionic acid
(S)-2-[(3′-{[(R)-1-(4-Chloro-3-methyl-phenyl)-propyl]-methyl-amino]-3,5-dimethyl-biphenyl-4-carbonyl}-methyl-amino)-propionic acid
(S)-2-({3′-[(S)-1-(4-Chloro-3-methyl-phenyl)-2,2,2-trifluoro-ethylamino]-3,5-dimethyl-biphenyl-4-carbonyl}-amino)-propionic acid
(S)-2-({3′-[(R)-1-(4-Chloro-3-methyl-phenyl)-propylamino]-3,5-dimethyl-biphenyl-4-carbonyl}-methyl-amino)-propionic acid
1-[(3′-{[(R)-1-(4-Chloro-3-methyl-phenyl)-propyl]-methyl-amino}-3,5-dimethyl-biphenyl-4-carbonyl)-amino]-cyclopropanecarboxylic acid
1-({3′-[(R)-1-(4-Chloro-3-methyl-phenyl)-propylamino]-3,5-dimethyl-biphenyl-4-carbonyl}-amino)-cyclopropanecarboxylic
1-[(3′-{[(R)-1-(4-Chloro-3-methyl-phenyl)-propyl]-methyl-amino}-3,5-dimethyl-biphenyl-4-carbonyl)-methyl-amino]-cyclopropanecarboxylic acid
1-({3′-[(R)-1-(4-Chloro-3-methyl-phenyl)-propylamino]-3,5-dimethyl-biphenyl-4-carbonyl}-methyl-amino)-cyclopropanecarboxylic acid
1-({3′-[(S)-1-(4-chloro-3-methyl-phenyl)-2,2,2-trifluoro-ethylamino]-3,5-dimethyl-biphenyl-4-carbonyl}-amino)-cyclopropanecarboxylic acid
(S)-2-({3′-[(R)-1-(4-Chloro-3-methyl-phenyl)-propylamino]-3,5-dimethyl-biphenyl-4-carbonyl}-methyl-amino)-propionic acid
(S)-3-Amino-2-({3′-[(R)-1-(4-chloro-3-methyl-phenyl)-ethylamino]-3,5-dimethyl-biphenyl-4-carbonyl}-amino)-propionic acid
1-({5′-[(R)-1-(4-Chloro-3-methyl-phenyl)-propylamino]-2′-fluoro-3,5-dimethyl-biphenyl-4-carbonyl}-amino)-cyclopropanecarboxylic acid
(S)-2-[(5′-{[(R)-1-(4-Chloro-3-methyl-phenyl)-propyl]-methyl-amino}-2′-fluoro-3,5-dimethyl-biphenyl-4-carbonyl)-amino]-propionic acid
(S)-2-({5′-[(R)-1-(4-Chloro-3-methyl-phenyl)-propylamino]-2′-fluoro-3,5-dimethyl-biphenyl-4-carbonyl}-methyl-amino)-propionic acid
(S)-2-[(5′-{[(R)-1-(4-Chloro-3-methyl-phenyl)-propyl]-methyl-amino]-2′-fluoro-3,5-dimethyl-biphenyl-4-carbonyl}-methyl-amino)-propionic acid

In another embodiment the present invention provides any one or more compound(s) according to formula (I) selected from the group consisting of:
2-({3′-[1-(3-Chloro-4-methoxy-phenyl)-ethylamino]-3,5-dimethyl-biphenyl-4-carbonyl}-amino)-propionic acid
2-({3′-[1-(4-Chloro-3-methyl-phenyl)-ethylamino]-3,5-dimethyl-biphenyl-4-carbonyl}-amino)-propionic acid
2-({3′-[1-(4-Chloro-3-fluoro-phenyl)-ethylamino]-3,5-dimethyl-biphenyl-4-carbonyl}-amino)-propionic acid
2-({3′-[1-(3,4-Dichlorophenyl)-ethylamino]-3,5-dimethyl-biphenyl-4-carbonyl}-amino)-propionic acid
2-({3′-[1-(3,4-Dimethylphenyl)-ethylamino]-3,5-dimethyl-biphenyl-4-carbonyl}-amino)-propionic acid
2-({3′-[1-(4-Chloro-3-methyl-phenyl)-ethylamino]-3,5-dimethyl-biphenyl-4-carbonyl}-amino)-propionic acid
2-({3′-[1-(4-Chloro-2,5-dimethyl-phenyl)-ethylamino]-3,5-dimethyl-biphenyl-4-carbonyl}-amino)-propionic acid
2-({3′-[1-(4-Chloro-3-trifluoromethyl-phenyl)-ethylamino]-3,5-dimethyl-biphenyl-4-carbonyl}-methyl-amino)-propionic acid
2-({3′-[1-(4-Fluoro-3-methoxy-phenyl)-ethylamino]-3,5-dimethyl-biphenyl-4-carbonyl}-amino)-propionic acid
2-({3′-[1-(4-Chloro-2-fluoro-5-methyl-phenyl)-ethylamino]-3,5-dimethyl-biphenyl-4-carbonyl}-amino)-propionic acid
2-({3′-[1-(4-Fluoro-3-trifluoromethoxy-phenyl)-ethylamino]-3,5-dimethyl-biphenyl-4-carbonyl}-amino)-propionic acid
2-({3′-[1-(3-Fluoro-4-methoxy-phenyl)-ethylamino]-3,5-dimethyl-biphenyl-4-carbonyl}-amino)-propionic acid
2-({3′-[1-(3-Fluoro-5-methyl-phenyl)-ethylamino]-3,5-dimethyl-biphenyl-4-carbonyl}-amino)-propionic acid
2-({3′-[1-(3,4-Dichloro-phenyl)-propylamino]-3,5-dimethyl-biphenyl-4-carbonyl}-amino)-propionic acid
2-({3′-[1-(4-Chloro-3-methyl-phenyl)-ethylamino]-3,5-dimethyl-biphenyl-4-carbonyl}-amino)-3-hydroxy-propionic acid
2-({3′-[1-(4-Chloro-3-methyl-phenyl)-ethylamino]-2′-fluoro-3,5-dimethyl-biphenyl-4-carbonyl}-amino)-propionic acid
2-({5′-[1-(4-Chloro-3-methyl-phenyl)-ethylamino]-2′-fluoro-3,5-dimethyl-biphenyl-4-carbonyl}-amino)-propionic acid
2-({5′-[1-(4-Chloro-3-methyl-phenyl)-propylamino]-2′-fluoro-3,5-dimethyl-biphenyl-4-carbonyl}-amino)-propionic acid
2-({3′-[1-(3,5-Dimethyl-phenyl)-ethylamino]-3,5-dimethyl-biphenyl-4-carbonyl}-amino)-propionic acid
2-({3′-[1-(4-Chloro-3,5-dimethyl-phenyl)-ethylamino]-3,5-dimethyl-biphenyl-4-carbonyl}-amino)-propionic acid
2-({3′-[1-(4-Methoxy-3-methyl-phenyl)-ethylamino]-3,5-dimethyl-biphenyl-4-carbonyl}-amino)-propionic acid
2-({3′-[1-(4-Methoxy-phenyl)-ethylamino]-3,5-dimethyl-biphenyl-4-carbonyl}-amino)-propionic acid
2-({3′-[1-(3,4-Dimethoxy-phenyl)-ethylamino]-3,5-dimethyl-biphenyl-4-carbonyl}-amino)-propionic acid
2-({3′-[1-(4-Methoxy-3-trifluoromethyl-phenyl)-ethylamino]-3,5-dimethyl-biphenyl-4-carbonyl}-amino)-propionic acid
2-({3′-[1-(3,5-Difluoro-4-methoxy-phenyl)-ethylamino]-3,5-dimethyl-biphenyl-4-carbonyl}-amino)-propionic acid
2-({3′-[1-(2-Fluoro-4-methoxy-phenyl)-ethylamino]-3,5-dimethyl-biphenyl-4-carbonyl}-amino)-propionic acid
2-({3′-[1-(3-Chloro-5-fluoro-4-methoxy-phenyl)-ethylamino]-3,5-dimethyl-biphenyl-4-carbonyl}-amino)-propionic acid
2-({3′-[1-(4-Methoxy-3,5-dimethyl-phenyl)-ethylamino]-3,5-dimethyl-biphenyl-4-carbonyl}-amino)-propionic acid
2-({3′-[1-(4-Chloro-3-methyl-phenyl)-ethylamino]-3-methyl-biphenyl-4-carbonyl}-amino)-propionic acid
2-({3′-[1-(4-Chloro-3-methyl-phenyl)-ethylamino]-3-methyl-biphenyl-4-carbonyl}-amino)-propionic acid
2-({3′-[(4-Chloro-3-methyl-phenyl)-ethylamino]-3-methyl-biphenyl-4-carbonyl}-amino)-propionic acid
2-({3′-[1-(4-Chloro-2,5-dimethyl-phenyl)-ethylamino]-3-methyl-biphenyl-4-carbonyl}-amino)-propionic acid
2-({3′-[1-(4-Chloro-3-methyl-phenyl)-ethylamino]-3-ethyl-biphenyl-4-carbonyl}-amino)-propionic acid
2-({3′-[(4-Chloro-3-methyl-phenyl)-ethylamino]-3-ethyl-biphenyl-4-carbonyl}-methyl-amino)-propionic acid
2-({3′-[(4-Chloro-3,5-dimethyl-phenyl)-ethylamino]-3-ethyl-biphenyl-4-carbonyl}-amino)-propionic acid
2-({3′-[1-(4-Chloro-3,5-dimethyl-phenyl)-ethylamino]-3-ethyl-biphenyl-4-carbonyl}-methyl-amino)-propionic acid
2-({3-Chloro-3′-[1-(4-chloro-3-methyl-phenyl)-ethylamino]-biphenyl-4-carbonyl}-amino)-propionic acid
2-({3-Chloro-3′-[1-(4-chloro-3-methyl-phenyl)-ethylamino]-biphenyl-4-carbonyl}-methyl-amino)-propionic acid
2-({3′-[(4-Chloro-3-methyl-phenyl)-ethylamino]-3-fluoro-biphenyl-4-carbonyl}-amino)-propionic acid
2-({3′-[1-(4-Chloro-3-methyl-phenyl)-ethylamino]-3-fluoro-biphenyl-4-carbonyl}-methyl-amino)-propionic acid
2-({3′-[1-(4-Chloro-3-methyl-phenyl)-ethylamino]-3,5-difluoro-biphenyl-4-carbonyl}-amino)-propionic acid
2-({3′-[1-(4-Chloro-3-methyl-phenyl)-ethylamino]-3,5-difluoro-biphenyl-4-carbonyl}-methyl-amino)-propionic acid
2-({3-Chloro-3′-[1-(4-chloro-3,5-dimethyl-phenyl)-ethylamino]-biphenyl-4-carbonyl}-amino)-propionic acid
2-({3-Chloro-3-[1-(4-chloro-3,5-dimethyl-phenyl)-ethylamino]-biphenyl-4-carbonyl}-methyl-amino)-propionic acid
2-({3′-[1-(4-Chloro-3,5-dimethyl-phenyl)-ethylamino]-3-fluoro-biphenyl-4-carbonyl}-amino)-propionic acid
2-({3′-[1-(4-Chloro-3,5-dimethyl-phenyl)-ethylamino]-3-fluoro-biphenyl-4-carbonyl}-methyl-amino)-propionic acid
2-({3′-[1-(4-Chloro-3,5-dimethyl-phenyl)-ethylamino]-3,5-difluoro-biphenyl-4-carbonyl}-amino)-propionic acid
2-({3′-[(4-Chloro-3,5-dimethyl-phenyl)-ethylamino]-3,5-difluoro-biphenyl-4-carbonyl}-methyl-amino)-propionic acid
2-({3′-[1-(4-Chloro-3-methyl-phenyl)-propylamino]-3-methyl-biphenyl-4-carbonyl}-methyl-amino)-propionic acid
2-({3′-[1-(4-Chloro-3,5-dimethyl-phenyl)-ethylamino]-3-methyl-biphenyl-4-carbonyl}-methyl-amino)-propionic acid
2-({3-Chloro-3′-[1-(4-chloro-3-methyl-phenyl)-propylamino]-biphenyl-4-carbonyl}-amino)-propionic acid
2-({3-Chloro-3′-[1-(4-chloro-3-methyl-phenyl)-propylamino]-biphenyl-4-carbonyl}-methyl-amino)-propionic acid
2-({3′-[1-(4-Chloro-3-methyl-phenyl)-ethylamino]-3-trifluoromethyl-biphenyl-4-carbonyl}-amino)-propionic acid
2-({3′-[1-(4-Chloro-3-methyl-phenyl)-ethylamino]-3-trifluoromethyl-biphenyl-4-carbonyl}-methyl-amino)-propionic acid
2-({3′-[1-(4-Chloro-3,5-dimethyl-phenyl)-ethylamino]-3-trifluoromethyl-biphenyl-4-carbonyl}-amino)-propionic acid
2-({3′-[1-(4-Chloro-3,5-dimethyl-phenyl)-ethylamino]-3-trifluoromethyl-biphenyl-4-carbonyl}-methyl-amino)-propionic acid
2-({3′-[1-(4-Chloro-3-methyl-phenyl)-propylamino]-3-trifluoromethyl-biphenyl-4-carbonyl}-amino)-propionic acid
2-({3′-[1-(4-Chloro-3-methyl-phenyl)-propylamino]-3-trifluoromethyl-biphenyl-4-carbonyl}-methyl-amino)-propionic acid
2-({3′-[1-(4-Chloro-3-methyl-phenyl)-ethylamino]-3,5-dimethyl-biphenyl-4-carbonyl}-amino)-acetic acid
2-({3′-[1-(4-Chloro-3-methyl-phenyl)-ethylamino]-3,5-dimethyl-biphenyl-4-carbonyl}-amino)-3-methoxy-propionic acid
6-Amino-2-({3′-[1-(4-chloro-3-methyl-phenyl)-ethylamino]-3,5-dimethyl-biphenyl-4-carbonyl}-amino)-hexanoic acid
2-({3′-[1-(4-Chloro-3-methyl-phenyl)-ethylamino]-3,5-dimethyl-biphenyl-4-carbonyl}-amino)-2-methyl-propionic acid
1-({3′-[1-(4-Chloro-3-methyl-phenyl)-ethylamino]-3,5-dimethyl-biphenyl-4-carbonyl}-amino)-cyclobutanecarboxylic acid
2-({3′-[1-(4-Chloro-3-methyl-phenyl)-ethylamino]-3,5-dimethyl-biphenyl-4-carbonyl}-methyl-amino)-propionic acid
2-({3′-[1-(4-Chloro-3-methyl-phenyl)-2,2,2-trifluoro-ethylamino]-3,5-dimethyl-biphenyl-4-carbonyl}-amino)-propionic acid
2-({3′-[1-(4-Chloro-3-methyl-phenyl)-2,2-difluoro-ethylamino]-3,5-dimethyl-biphenyl-4-carbonyl}-amino)-propionic acid
2-({3′-[1-(4-Chloro-3-methyl-phenyl)-ethylamino]-3,5-dimethyl-biphenyl-4-carbonyl}-methyl-amino)-propionic acid
2-({3′-[(4-Chloro-phenyl)-ethylamino]-3-methyl-biphenyl-4-carbonyl}-methyl-amino)-propionic acid
2-({3′-[(3,4-Dichloro-phenyl)-ethylamino]-3-methyl-biphenyl-4-carbonyl}-methyl-amino)-propionic acid
3-({3′-[1-(4-Chloro-3-methyl-phenyl)-ethylamino]-3,5-dimethyl-biphenyl-4-carbonyl}-amino)-azetidine-3-carboxylic acid
2-[(3′-{[1-(4-Chloro-3-methyl-phenyl)-ethyl]-methyl-amino}-3,5-dimethyl-biphenyl-4-carbonyl)-amino]-propionic acid
1-({3′-[1-(4-Chloro-3-methyl-phenyl)-ethylamino]-3,5-dimethyl-biphenyl-4-carbonyl}-methyl-amino)-cyclopropanecarboxylic acid
2-({3′-[(4-Chloro-3-methyl-phenyl)-1-methyl-ethylamino]-3,5-dimethyl-biphenyl-4-carbonyl}-amino)-propionic acid
2-({3′-[1-(4-Chloro-3-methyl-phenyl)-1-methyl-ethylamino]-3,5-dimethyl-biphenyl-4-carbonyl}-methyl-amino)-propionic acid
1-({3′-[1-(4-Chloro-3-methyl-phenyl)-1-methyl-ethylamino]-3,5-dimethyl-biphenyl-4-carbonyl}-amino)-cyclopropanecarboxylic acid
1-({3′-[1-(4-Chloro-3-methyl-phenyl)-1-methyl-ethylamino]-3,5-dimethyl-biphenyl-4-carbonyl}-methyl-amino)-cyclopropanecarboxylic acid
2-({5′41-(4-Chloro-3-methyl-phenyl)-propylamino}-2′-fluoro-3-methyl-biphenyl-4-carbonyl)-methyl-amino)-propionic acid
2-({3-Chloro-5′-[1-(4-chloro-3-methyl-phenyl)-ethylamino]-2′-fluoro-biphenyl-4-carbonyl}-methyl-amino)-propionic acid
2-({3-Chloro-5′-[1-(4-chloro-3-methyl-phenyl)-propylamino]-2′-fluoro-biphenyl-4-carbonyl}-methyl-amino)-propionic acid
2-({5′-[1-(4-Chloro-3-methyl-phenyl)-propylamino]-2′-fluoro-3-trifluoromethyl-biphenyl-4-carbonyl}-methyl-amino)-propionic acid
2-({3′-[1-(4-Chloro-3-methyl-phenyl)-2-methyl-propylamino]-3,5-dimethyl-biphenyl-4-carbonyl}-amino)-propionic acid
2-({3′-[(4-Chloro-3-methyl-phenyl)-2,2,2-trifluoro-ethylamino]-3-trifluoromethyl-biphenyl-4-carbonyl}-methyl-amino)-propionic acid
2-({3′-[1-(4-Chloro-3-methyl-phenyl)-2,2,2-trifluoro-ethylamino]-3-trifluoromethyl-biphenyl-4-carbonyl}-amino)-propionic acid
1-({3′-[1-(4-Chloro-3-methyl-phenyl)-2,2,2-trifluoro-ethylamino]-3-trifluoromethyl-biphenyl-4-carbonyl}-methyl-amino)-cyclopropanecarboxylic acid
2-({3-Chloro-3′-[1-(4-chloro-3-methyl-phenyl)-2,2,2-trifluoro-ethylamino]-biphenyl-4-carbonyl}-amino)-propionic acid
1-({3-Chloro-3′-[1-(4-chloro-3-methyl-phenyl)-2,2,2-trifluoro-ethylamino]-biphenyl-4-carbonyl}-amino)-cyclopropanecarboxylic acid
2-({3-Chloro-3′-[(S)-1-(4-chloro-3-methyl-phenyl)-2,2,2-trifluoro-ethylamino]-biphenyl-4-carbonyl}-methyl-amino)-propionic acid
2-({3-Chloro-3′-[1-(4-chloro-3-methyl-phenyl)-1-methyl-ethylamino]-biphenyl-4-carbonyl}-amino)-propionic acid
2-({3-Chloro-3′-[1-(4-chloro-3-methyl-phenyl)-1-methyl-ethylamino]-biphenyl-4-carbonyl}-methyl-amino)-propionic acid
1-({3′-[1-(4-Chloro-3,5-dimethyl-phenyl)-ethylamino]-3,5-dimethyl-biphenyl-4-carbonyl}-amino)-cyclopropanecarboxylic acid
2-({3′-[1-(4-Chloro-3,5-dimethyl-phenyl)-ethylamino]-3,5-dimethyl-biphenyl-4-carbonyl}-amino)-propionic acid
2-({3′-[1-(4-Chloro-3,5-dimethyl-phenyl)-propylamino]-3,5-dimethyl-biphenyl-4-carbonyl}-amino)-propionic acid
1-({3′-[1-(4-Chloro-3,5-dimethyl-phenyl)-propylamino]-3,5-dimethyl-biphenyl-4-carbonyl}-amino)-cyclopropanecarboxylic acid
2-({3′-[1-(4-Chloro-3-methyl-phenyl)-2,2-difluoro-ethylamino]-3,5-dimethyl-biphenyl-4-carbonyl}-methyl-amino)-propionic acid
2-[(3′-{[1-(4-Chloro-3-methyl-phenyl)-propyl]-methyl-amino}-3,5-dimethyl-biphenyl-4-carbonyl)-amino]-propionic acid
2-[(3′-{[1-(4-Chloro-3-methyl-phenyl)-propyl]-methyl-amino}-3,5-dimethyl-biphenyl-4-carbonyl)-methyl-amino]-propionic acid
2-({3′-[(S)-1-(4-Chloro-3-methyl-phenyl)-2,2,2-trifluoro-ethylamino]-3,5-dimethyl-biphenyl-4-carbonyl}-amino)-propionic acid
2-({3′-[1-(4-Chloro-3-methyl-phenyl)-propylamino]-3,5-dimethyl-biphenyl-4-carbonyl}-methyl-amino)-propionic acid
1-[(3′-{[1-(4-Chloro-3-methyl-phenyl)-propyl]-methyl-amino}-3,5-dimethyl-biphenyl-4-carbonyl)-amino]-cyclopropanecarboxylic acid
1-({3′-[(4-Chloro-3-methyl-phenyl)-propylamino]-3,5-dimethyl-biphenyl-4-carbonyl}-amino)-cyclopropanecarboxylic
1-[(3′-{[1-(4-Chloro-3-methyl-phenyl)-propyl]-methyl-amino}-3,5-dimethyl-biphenyl-4-carbonyl)-methyl-amino]-cyclopropanecarboxylic acid
1-({3′-[1-(4-Chloro-3-methyl-phenyl)-propylamino]-3,5-dimethyl-biphenyl-4-carbonyl}-methyl-amino)-cyclopropanecarboxylic acid
1-({3′-[1-(4-Chloro-3-methyl-phenyl)-2,2,2-trifluoro-ethylamino]-3,5-dimethyl-biphenyl-4-carbonyl}-amino)-cyclopropanecarboxylic acid
2-({3′-[1-(4-Chloro-3-methyl-phenyl)-propylamino]-3,5-dimethyl-biphenyl-4-carbonyl}-methyl-amino)-propionic acid
3-Amino-2-({3′-[1-(4-chloro-3-methyl-phenyl)-ethylamino]-3,5-dimethyl-biphenyl-4-carbonyl}-amino)-propionic acid
1-({5′-[1-(4-Chloro-3-methyl-phenyl)-propylamino]-2′-fluoro-3,5-dimethyl-biphenyl-4-carbonyl}-amino)-cyclopropanecarboxylic acid
2-[(5′-{[1-(4-Chloro-3-methyl-phenyl)-propyl]-methyl-amino}-2′-fluoro-3,5-dimethyl-biphenyl-4-carbonyl)-amino]-propionic acid
2-({5′-[1-(4-Chloro-3-methyl-phenyl)-propylamino]-2′-fluoro-3,5-dimethyl-biphenyl-4-carbonyl}-methyl-amino)-propionic acid
2-[(5-{[1-(4-Chloro-3-methyl-phenyl)-propyl]-methyl-amino}-2′-fluoro-3,5-dimethyl-biphenyl-4-carbonyl)-methyl-amino]-propionic acid

### EXPERIMENTAL SECTION

Abbreviations:
BOC: t-Butyloxycarbonyl
DCM: Dichloromethane
DIPEA: Ethyl-diisopropyl-amine, Hünig's base, DIEA
DME: 1,2-Dimethoxy-ethane
DMF: N,N-Dimethyl formamide
Ether: Diethylether, Ethoxy-ethane
EtOAc: Acetic acid ethyl ester
EtOH: Ethanol
HATU: N-[(Dimethylamino)-1H-1,2,3-triazolo[4,5-b]pyridin-1-ylmethylene]-N-methylmethanaminium Hexafluorophosphate N-Oxide
MeOH: Methanol
min: minutes
NMP: 2-methylpyrrolidone
TBME: 2-Methoxy-2-methyl-propane
TBTU: O-Benzotriazol-1-yl-N,N,N′,N′-tetramethyluronium tetrafluoroborate
TFA: Trifluoro-acetic acid
THF: Tetrahydrofuran
rt: Retention time

¹H-NMR spectra were recorded on a Varian Gemini 400 MHz or a Bruker 360 MHz NMR spectrometer. Significant peaks are tabulated in the order: multiplicity (s, singlet; d, doublet; t, triplet; q, quartet; m, multiplet; br, broad; v, very) and number of protons. Electron Spray Ionization (ESI) mass spectra were recorded on a Hewlett Packard 5989A or an Agilent 1100 Series mass spectrometer. Mass spectrometry results are reported as the ratio of mass over charge.

Detailed analytical HPLC chromatography methods referred to in the preparations and Examples below are outlined as follows:

### LC/MS Method 1:

Waters Acquity UPLC instrument equipped with diode array detector, Waters ZQ2000 mass spectrometer and Waters Xterra C18 (2.5 μm) 3×30 mm column. Peak detection is reported at 210 nm wavelength.

Solvent A: Water containing 5% (acetonitrile with 0.05% formic acid).

Solvent B: Acetonitrile containing 0.05% formic acid.

Gradient:

| | Solvent | Solvent | Flow rate |
|---|---|---|---|
| Time [minutes] | A [%] | B [%] | [mL/min] |
| 0 | 90 | 10 | 1.4 |
| 5 | 5 | 95 | 1.6 |
| 5.5 | 5 | 95 | 2.4 |
| 5.6 | 90 | 10 | 2.4 |
| 6 | 90 | 2 | 1.4 |

### LC/MS Method 2:

Waters Acquity UPLC instrument equipped with diode array detector, Waters SQD Single Stage Quadrupole mass spectrometer or Waters ZQ2000 mass spectrometer and Waters Acquity HSS T3 (1.8 μm) 2.1×50 mm column. Peak detection is reported at 210 nm wavelength.

Solvent A: Water containing 3 mM ammonium acetate and 0.05% formic acid.

Solvent B: Acetonitrile containing 0.04% formic acid.

Flow rate at 0.6 mL/minute

Gradient:

| Time [minutes] | Solvent A [%] | Solvent B [%] |
|---|---|---|
| 0 | 98 | 2 |
| 5 | 2 | 98 |
| 5.5 | 2 | 98 |
| 5.6 | 98 | 2 |
| 6 | 98 | 2 |

### UPLC Method:

Waters Acquity UPLC instrument equipped with diode array detector and Waters Acquity UPLC® BEH C18 (1.7 μm) 2.1×50 mm column. Peak detection is reported at 210 nm wavelength.

Solvent A: water (1800 mL), acetonitrile (200 mL), tetramethylammonium hydroxide (40 mL, 10% in water), phosphoric acid (4 mL)

Solvent B: water (500 mL), acetonitrile (1500 mL), tetramethylammonium hydroxide (40 mL, 10% in water), phosphoric acid (4 mL)

Flow rate at 0.75 mL/minute

Gradient:

| Time [minutes] | Solvent A [%] | Solvent B [%] |
|---|---|---|
| 0 | 95 | 5 |
| 2 | 5 | 95 |
| 2.7 | 5 | 95 |
| 2.75 | 95 | 5 |
| 3 | 95 | 5 |

### Preparative Chiral Separation:

Method A: Separation was performed using a Chiralpak AD-H 250×30 mm (5 μm) and n-hexane/EtOH or n-heptane/EtOH as mobile phase with a flow of 25 ml/min and UV detection (220 nm).

Method B: Separation was performed using a Chiralcel OJ 10×50 cm (20 μm) and n-heptane/EtOH as mobile phase with a flow of 100 mL/min and UV detection (220 nm).

### Analytical Chiral HPLC:

Method C: Analysis was performed using a Chiralpak AD-H 250×4.6 mm (5 μm) and n-hexane/EtOH as mobile phase with a flow of 1 mL/min and UV detection (220 nm).

Method D: Analysis was performed using a Chiralpak AD-H 250×4.6 mm (20 μm) and n-heptane/EtOH as mobile phase with a flow of 1 mL/min and UV detection (220 nm).

Method E: Analysis was performed using a Chiralcel OJ 250×4.6 mm (5 μm) and n-heptane/EtOH/MeOH as mobile phase with a flow of 0.9 mL/min and UV detection (220 nm).

All reagents, starting materials and intermediates utilized in these Examples are available from commercial sources or are readily prepared by methods known to those skilled in the art.

### Synthesis of Biaryl Benzylamine Derivatives

Agents of the invention may be prepared by a reaction sequence involving Suzuki-type coupling of an appropriate boronic acid or ester with an appropriate aryl halide, coupling with an appropriate amino ester, and reductive amination with an appropriate ketone followed by a deprotection step as shown in Scheme 1 below:

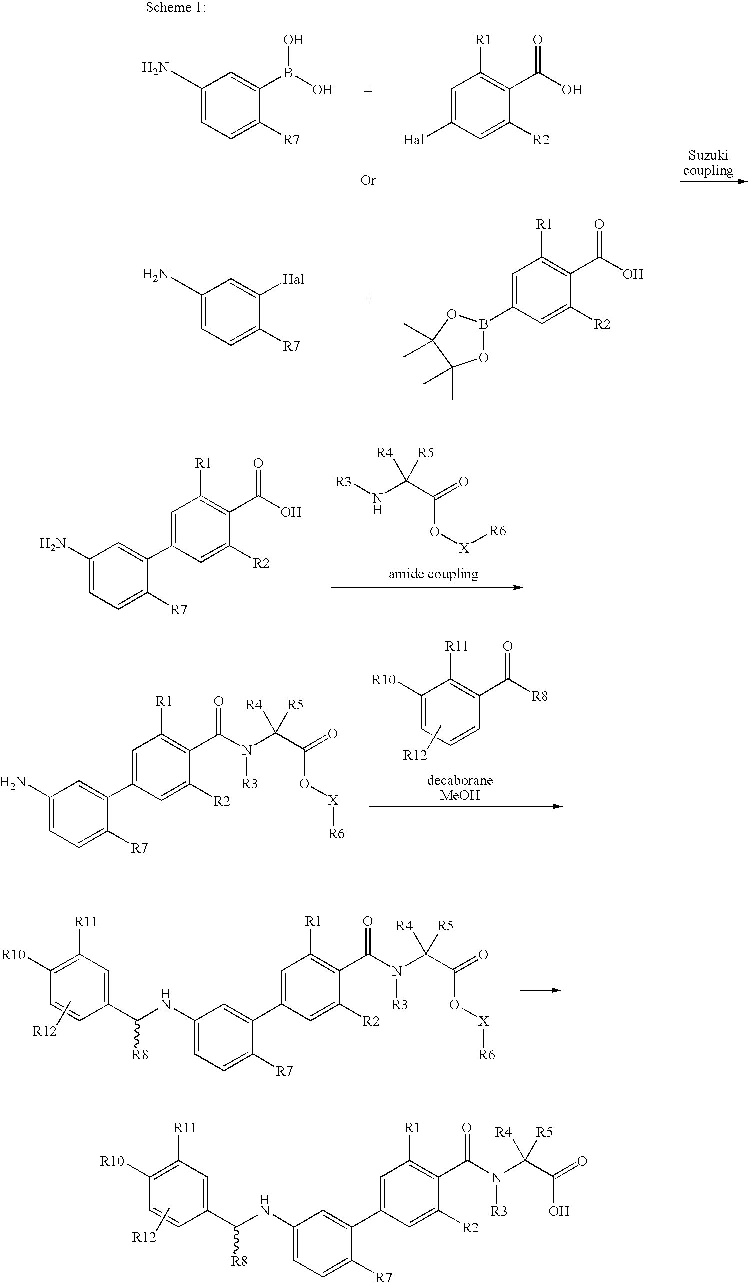

### Example EX1

### (S)-2-({3′-[1-(3-Chloro-4-methoxy-phenyl)-ethylamino]-3,5-dimethyl-biphenyl-4-carbonyl}-amino)-propionic acid

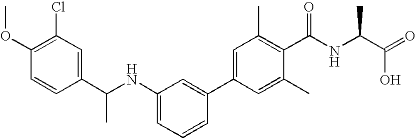

### (1) 3′-Amino-3,5-dimethyl-biphenyl-4-carboxylic acid, INT1

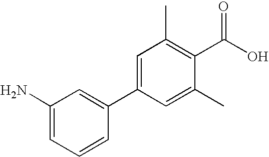

To a mixture of 4-bromo-2,6-dimethyl-benzoic acid (1.66 g, 7.23 mmol) and tetrakis-triphenylphosphinopalladium (25 mg, 0.022 mmol) in DME (200 mL) and aqueous sodium bicarbonate solution (10%, 45 mL, 50.6 mmol) was added (3-aminophenyl)-boronic acid (1.09 g, 7.95 mmol). The mixture was heated to 100° C. for 60 minutes. Upon cooling a brownish oily layer was formed which was carefully decanted. The solvents were then evaporated. Water was added and the mixture was washed with ether. The pH of the aqueous layer was adjusted to about 4 with 2M HCl upon which a slightly sticky solid precipitates. The solid was filtered off, re-dissolved in ethyl acetate and dried over sodium sulfate. Filtration and evaporation gave the title compound INT1 as a beige powder.

LC/MS method 2: MS (ESI): 242 [M+H]⁺, rt=1.67 min. ¹H-NMR (DMSO-d₆): δ (ppm) 7.27 (s, 2H), 7.1 (t, 1H), 6.84 (br s, 1H), 6.76 (d, 1H), 6.58 (m, 1H), 3.35 (br s, 2H), 2.33 (s, 3H).

### (2) (S)-2-[(3′-Amino-3,5-dimethyl-biphenyl-4-carbonyl)-amino]-propionic acid methyl ester, INT2

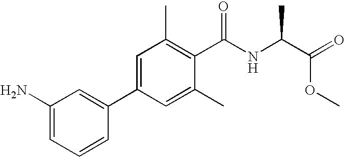

To a solution of INT1 (339 mg, 1.405 mmol) and alanine methyl ester hydrochloride (294 mg, 2.108 mmol) in DMF (7 mL) was added DIPEA (545 mg, 4.215 mmol) followed by TBTU (541 mg, 1.686 mmol) and the resulting mixture was stirred at room temperature overnight. The DMF was evaporated under reduced pressure and the residue dissolved in EtOAc. The organic layer was washed with 5% aqueous sodium bicarbonate, brine/water (1:1) and brine, dried over sodium sulfate, filtered and concentrated. The residue was purified by chromatography on silica gel (cyclohexane/EtOAc) to give INT2 as a white solid.

LC/MS method 1: MS (ESI): 327 [M+H]⁺, rt=0.75 min. ¹H-NMR (CDCl₃): δ (ppm) 7.24-7.18 (m, 3H), 6.99-6.91 (m, 1H), 6.86 (d, 1H), 6.71-6.61 (m, 1H), 6.26 (d, 1H), 4.93-4.79 (m, 1H), 3.80 (s, 3H), 3.74 (br s, 2H), 2.38 (s, 6H), 1.54 (d, 3H).

### (3) (S)-2-({3′-[1-(3-Chloro-4-methoxy-phenyl)-ethylamino]-3,5-dimethyl-biphenyl-4-carbonyl}-amino)-propionic acid methyl ester, INT3

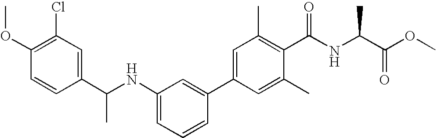

To a solution of INT2 (100 mg, 0.306 mmol) and 1-(3-chloro-4-methoxy-phenyl)-ethanone (62 mg, 0.337 mmol) in MeOH (3 mL) was added decaborane (18.7 mg, 0.153 mmol) and the resulting mixture was stirred under argon overnight. The solvent was evaporated and the residue was purified by chromatography on silica gel (cyclohexane/EtOAc) to give INT3 as a white solid.

(CDCl₃): δ (ppm) 7.40 (d, 1H), 7.24 (dd, 1H), 7.14 (t, 1H), 7.12 (s, 2H), 6.89 (d, 1H), 6.84 (d, 1H), 6.68 (t, 1H), 6.47 (dd, 1H), 6.24 (d, 1H), 4.90-4.82 (m, 1H), 4.46 (q, 1H), 3.88 (s, 3H), 3.79 (s, 3H), 2.36 (s, 6H), 1.53 (d, 3H), 1.51 (d, 3H).

### (4) (S)-2-({3′-[1-(3-Chloro-4-methoxy-phenyl)-ethylamino]-3,5-dimethyl-biphenyl-4-carbonyl}-amino)-propionic acid

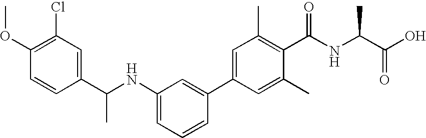

To a solution of INT3 (131 mg, 0.265 mmol) in THF/water (2.6 mL, 2:1) was added LiOH hydrate (22.2 mg, 0.529 mmol) and the resulting mixture was stirred at room temperature for 3 hours. The reaction mixture was diluted with EtOAc and water. 1M HCl (0.529 mL) was then added and the organic layer was separated, washed with brine, dried over sodium sulfate, filtered and concentrated. The residue was purified by chromatography on silica gel (cyclohexane/EtOAc) to give Example EX1 as a white solid.

LC/MS Method 2: MS (ESI): 481 [M+H]⁺, rt=2.93 min. ¹H-NMR (DMSO-d₆): δ (ppm) 12.46 (br s, 1H), 8.56 (d, 1H), 7.42 (s, 1H), 7.28 (s, 1H), 7.09 (s, 2H), 7.06 (d, 1H), 6.70 (s, 1H), 6.69 (d, 1H), 6.46 (d, 1H), 6.22 (d, 1H), 4.52-4.45 (m, 1H), 4.40-4.33 (m, 1H), 3.78 (s, 3H), 2.26 (s, 6H), 1.40 (d, 3H), 1.33 (d, 3H).

### Example EX2

### (S)-2-({3′-[1-(4-Chloro-3-methyl-phenyl)-ethylamino]-3,5-dimethyl-biphenyl-4-carbonyl}-amino)-propionic acid

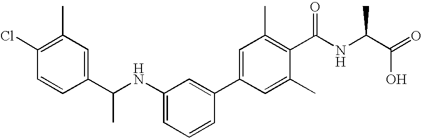

### (1) 1-(4-Chloro-3-methyl-phenyl)-ethanone, INT4

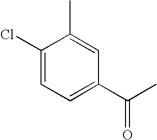

To a solution 4-chloro-3-methylbenzoic acid (38.4 g, 200 mmol) in THF (2 L) at 0° C. was added dropwise MeLi (500 mL, 1.6 M in ether) and the resulting mixture was stirred at room temperature for 4 hours. The mixture was then poured slowly on cooled 2N HCl (830 mL), the organic layer was separated and the aqueous layer was extracted with EtOAc (300 mL). The combined organic layers were washed with brine, dried over sodium sulfate, filtered and concentrated. The residue was purified by chromatography on silica gel (cyclohexane/EtOAc) to give INT4 as a pale yellow liquid.

LC/MS Method 1: MS (ESI): 169 [M+H]⁺, rt=1.23 min. ¹H-NMR (CDCl₃): δ (ppm) 7.82 (d, 1H), 7.71 (dd, 1H), 7.43 (d, 1H), 2.58 (s, 3H), 2.43 (s, 3H).

### (2) (S)-2-({3′-[1-(4-Chloro-3-methyl-phenyl)-ethylamino]-3,5-dimethyl-biphenyl-4-carbonyl}-amino)-propionic acid methyl ester, INT5

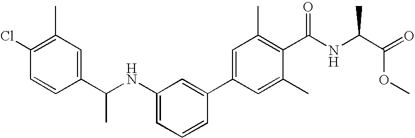

To a solution of INT2 (700 mg, 2.15 mmol) and INT4 (398 mg, 2.36 mmol) in MeOH (21 mL) was added decaborane (131 mg, 1.07 mmol) and the resulting mixture was stirred under argon overnight. The solvent was evaporated and the residue was purified by chromatography on silica gel (cyclohexane/EtOAc) to give INT5 as a white solid.

(CDCl₃): δ (ppm) 7.28 (d, 1H), 7.25 (d, 1H), 7.17-7.12 (m, 2H), 7.10 (s, 2H), 6.84 (d, 1H), 6.67 (t, 1H), 6.46 (dd, 1H), 6.22 (d, 1H), 4.90-4.82 (m, 1H), 4.46 (q, 1H), 3.79 (s, 3H) 2.36 (s, 9H), 1.53 (d, 3H), 1.51 (d, 3H).

### (3) (S)-2-({3′-[1-(4-Chloro-3-methyl-phenyl)-ethylamino]-3,5-dimethyl-biphenyl-4-carbonyl}-amino)-propionic acid

To a solution of INT5 (200 mg, 0.418 mmol) in THF/water (4 mL, 2:1) was added LiOH hydrate (35.0 mg, 0.836 mmol) and the resulting mixture was stirred at room temperature for 3 hours. The reaction mixture was diluted with EtOAc and water. 1M HCl (0.836 mL) was then added and the organic layer was separated, washed with brine, dried over sodium sulfate, filtered and concentrated. The residue was purified by chromatography on silica gel (cyclohexane/EtOAc) to give Example EX2 as a white solid.

LC/MS Method 2: MS (ESI): 465 [M+H]⁺, rt=3.20 min. ¹H-NMR (DMSO-d₆): δ (ppm) 12.45 (br s, 1H), 8.57 (d, 1H), 7.36 (d, 1H), 7.30 (d, 1H), 7.21 (d, 1H), 7.07 (s, 2H), 7.02 (t, 1H), 6.70 (s, 1H), 6.68 (d, 1H), 6.49 (d, 1H), 4.51-4.44 (m, 1H), 4.40-4.33 (m, 1H), 2.29 (s, 3H), 2.25 (s, 6H), 1.40 (d, 3H), 1.32 (d, 3H).

### Example EX3

### (S)-2-({3′-[(R)-1-(4-Chloro-3-methyl-phenyl)-ethylamino]-3,5-dimethyl-biphenyl-4-carbonyl}-amino)-propionic acid

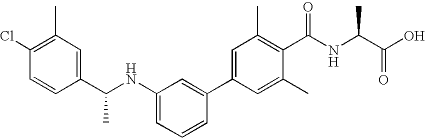

### (1) (S)-2-({3′-[(R)-1-(4-Chloro-3-methyl-phenyl)-ethylamino]-3,5-dimethyl-biphenyl-4-carbonyl}-amino)-propionic acid methyl ester, INT6

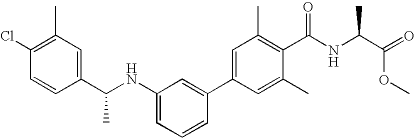

The title compound was obtained by preparative chiral separation of INT5 (Method A). Chiral HPLC method C: rt=9.07 min, n-hexane/EtOH (80:20).

¹H-NMR (CDCl₃): δ (ppm) 7.28 (d, 1H), 7.25 (d, 1H), 7.17-7.12 (m, 2H), 7.10 (s, 2H), 6.84 (d, 1H), 6.67 (t, 1H), 6.46 (dd, 1H), 6.22 (d, 1H), 4.90-4.82 (m, 1H), 4.46 (q, 1H), 3.79 (s, 3H) 2.36 (s, 9H), 1.53 (d, 3H), 1.51 (d, 3H).

### (2) (S)-2-({3′-[(R)-1-(4-Chloro-3-methyl-phenyl)-ethylamino]-3,5-dimethyl-biphenyl-4-carbonyl}-amino)-propionic acid

To a solution of INT6 (230 mg, 0.480 mmol) in THF/water (4.5 mL, 2:1) was added LiOH hydrate (40.3 mg, 0.960 mmol) and the resulting mixture was stirred at room temperature for 3 hours. The reaction mixture was diluted with EtOAc and water. 1M HCl (0.960 mL) was then added and the organic layer was separated, washed with brine, dried over sodium sulfate, filtered and concentrated. The residue was purified by chromatography on silica gel (cyclohexane/EtOAc) to give Example EX3 as a white solid.

LC/MS Method 2: MS (ESI): 465 [M+H]⁺, rt=3.19 min. ¹H-NMR (DMSO-d₆): δ (ppm) 12.45 (br s, 1H), 8.57 (d, 1H), 7.36 (d, 1H), 7.30 (d, 1H), 7.21 (d, 1H), 7.07 (s, 2H), 7.02 (t, 1H), 6.70 (s, 1H), 6.68 (d, 1H), 6.49 (d, 1H), 4.51-4.44 (m, 1H), 4.40-4.33 (m, 1H), 2.29 (s, 3H), 2.25 (s, 6H), 1.40 (d, 3H), 1.32 (d, 3H).

### Example EX4

### (S)-2-({3′-[(S)-1-(4-Chloro-3-methyl-phenyl)-ethylamino]-3,5-dimethyl-biphenyl-4-carbonyl}-amino)-propionic acid

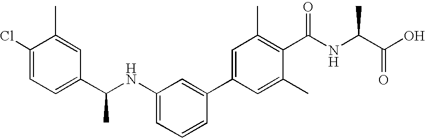

### (1) (S)-2-({3′-[(S)-1-(4-Chloro-3-methyl-phenyl)-ethylamino]-3,5-dimethyl-biphenyl-4-carbonyl}-amino)-propionic acid methyl ester, INT7

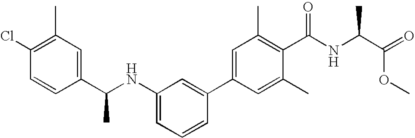

The title compound was obtained by preparative chiral separation of INT5 (Method A). Chiral HPLC: rt=12.11 min, n-hexane/EtOH (80:20).

¹H-NMR (CDCl₃): b (ppm) 7.28 (d, 1H), 7.25 (d, 1H), 7.17-7.12 (m, 2H), 7.10 (s, 2H), 6.84 (d, 1H), 6.67 (t, 1H), 6.46 (dd, 1H), 6.22 (d, 1H), 4.90-4.82 (m, 1H), 4.46 (q, 1H), 3.79 (s, 3H) 2.36 (s, 9H), 1.53 (d, 3H), 1.51 (d, 3H).

### (2) (S)-2-({3′-[(S)-1-(4-Chloro-3-methyl-phenyl)-ethylamino]-3,5-dimethyl-biphenyl-4-carbonyl}-amino)-propionic acid

To a solution of INT7 (200 mg, 0.418 mmol) in THF/water (4 mL, 2:1) was added LiOH hydrate (35.0 mg, 0.836 mmol) and the resulting mixture was stirred at room temperature for 3 hours. The reaction mixture was diluted with EtOAc and water. 1M HCl (0.836 mL) was then added and the organic layer was separated, washed with brine, dried over sodium sulfate, filtered and concentrated. The residue was purified by chromatography on silica gel (cyclohexane/EtOAc) to give Example EX4 as a white solid.

LC/MS Method 2: MS (ESI): 465 [M+H]⁺, rt=3.19 min. ¹H-NMR (DMSO-d₆): δ (ppm) 12.45 (br s, 1H), 8.57 (d, 1H), 7.36 (d, 1H), 7.30 (d, 1H), 7.21 (d, 1H), 7.07 (s, 2H), 7.02 (t, 1H), 6.70 (s, 1H), 6.68 (d, 1H), 6.49 (d, 1H), 4.51-4.44 (m, 1H), 4.40-4.33 (m, 1H), 2.29 (s, 3H), 2.25 (s, 6H), 1.40 (d, 3H), 1.32 (d, 3H).

### Example EX5

### (S)-2-({3′-[1-(4-Chloro-3-methyl-phenyl)-propylamino]-3,5-dimethyl-biphenyl-4-carbonyl}-amino)-propionic acid

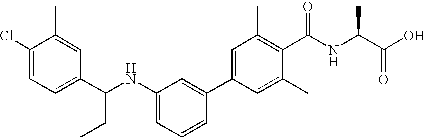

### (1) 1-(4-Chloro-3-methyl-phenyl)-propan-1-one, INT8

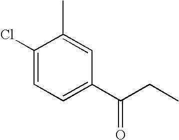

To a solution 4-chloro-3-methylbenzoic acid (8.31 g, 48.7 mmol) in DCM (50 mL) was added DMF (175 μL) and thionyl chloride (35.5 mL, 487 mmol) and the resulting mixture was refluxed for 1 hour. After cooling down, the mixture was evaporated to dryness under reduced pressure and taken up in THF (50 mL). The resulting solution was cooled down to 0° C. and triethylamine (13.5 mL, 97 mmol) was added followed by N,O-dimethylhydroxylamine hydrochloride (5.7 g, 58.4 mmol) and the resulting mixture was stirred at room temperature overnight. The crude was diluted in dichloromethane and washed with 1M KHSO₄, saturated aqueous sodium bicarbonate and brine, dried over sodium sulfate and concentrated. The resulting crude Weinreb amide (10.4 g, 48.7 mmol) was taken up in THF (440 mL) and cooled down to 0° C. under an argon atmosphere. EtMgBr (1N in TBME, 97 mmol) was then slowly added and the resulting mixture was stirred for 2 hours at 0° C. The reaction was then quenched with a saturated ammonium chloride solution. The THF was evaporated and the product was then extracted with EtOAc and washed with brine. The residue was purified by chromatography on silica gel (cyclohexane/EtOAc) to give INT8 as a yellow solid.

¹H-NMR (CDCl₃): δ (ppm) 7.93 (d, 1H), 7.76 (dd, 1H), 7.55 (d, 1H), 3.01 (q, 2H), 2.39 (s, 3H) 1.06 (t, 3H).

### (2) (S)-2-({3′-[1-(4-Chloro-3-methyl-phenyl)-propylamino]-3,5-dimethyl-biphenyl-4-carbonyl}-amino)-propionic acid methyl ester, INT9

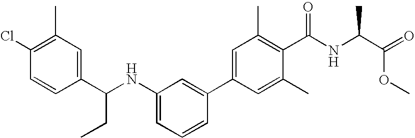

To a solution of INT2 (700 mg, 2.15 mmol) and INT8 (470 mg, 2.57 mmol) in MeOH (20 mL) was added decaborane (157 mg, 1.29 mmol) and the resulting mixture was stirred under argon overnight. The solvent was evaporated and the residue was purified by chromatography on silica gel (cyclohexane/EtOAc) to give INT9 as a white solid.

LC/MS Method 2: MS (ESI): 493 [M+H]⁺, rt=3.30 min. ¹H-NMR (DMSO-d₆): δ (ppm) 8.75 (d, 1H), 7.37 (s, 1H), 7.33 (d, 1H), 7.22 (d, 1H), 7.11 (s, 2H), 7.03 (t, 1H), 6.74 (s, 1H), 6.70 (d, 1H), 6.47 (d, 1H), 6.24 (d, 1H), 4.48-4.42 (m, 1H), 4.25 (q, 1, 3.66 (s, 3H), 2.29 (s, 3H), 2.26 (s, 6H), 1.84-1.75 (m, 1H), 1.70-1.63 (m, 1H), 1.33 (d, 3H), 0.90 (t, 3H).

### (3) (S)-2-({3′-[1-(4-Chloro-3-methyl-phenyl)-propylamino]-3,5-dimethyl-biphenyl-4-carbonyl}-amino)-propionic acid

To a solution of INT9 (117 mg, 0.237 mmol) in THF/water (2.5 mL, 2:1) was added LiOH hydrate (19.9 mg, 0.475 mmol) and the resulting mixture was stirred at room temperature for 3 hours. The reaction mixture was diluted with EtOAc and water. 1M HCl (0.475 mL) was then added and the organic layer was separated, washed with brine, dried over sodium sulfate, filtered and concentrated. The residue was purified by chromatography on silica gel (cyclohexane/EtOAc) to give Example EX5 as a white solid.

LC/MS Method 2: MS (ESI): 479 [M+H]⁺, rt=2.98 min. ¹H-NMR (DMSO-d₆): δ (ppm) 12.51 (br s, 1H), 8.75 (d, 1H), 7.37 (s, 1H), 7.32 (d, 1H), 7.22 (dd, 1H), 7.10 (s, 2H), 7.03 (t, 1H), 6.74 (s, 1H), 6.69 (d, 1H), 6.47 (d, 1H), 6.23 (d, 1H), 4.40-4.34 (m, 1H), 4.25 (q, 1H), 2.29 (s, 3H), 2.26 (s, 6H), 1.83-1.75 (m, 1H), 1.70-1.62 (m, 1H), 1.32 (d, 3H), 0.90 (t, 3H).

### Example EX6

### (S)-2-({3′-[(R)-1-(4-Chloro-3-methyl-phenyl)-propylamino]-3,5-dimethyl-biphenyl-4-carbonyl}-amino)-propionic acid

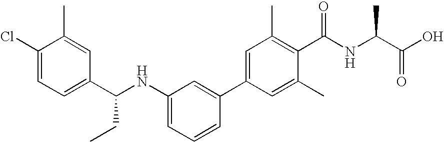

### (1) (S)-2-({3′-[(R)-1-(4-Chloro-3-methyl-phenyl)-propylamino]-3,5-dimethyl-biphenyl-4-carbonyl}-amino)-propionic acid methyl ester, INT10

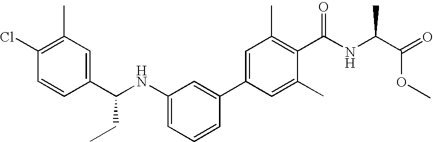

The title compound was obtained by preparative chiral separation of INT9 (Method A). Chiral HPLC method D: rt=8.86 min, heptane/EtOH (80:20).

LC/MS Method 2: MS (ESI): 493 [M+H]⁺, rt=3.30 min. ¹H-NMR (DMSO-d₆): δ (ppm) 8.69 (d, 1H), 7.34 (s, 1H), 7.28 (d, 1H), 7.19 (d, 1H), 7.08 (s, 2H), 7.00 (t, 1H), 6.72 (s, 1H), 6.67 (d, 1H), 6.45 (d, 1H), 6.19 (d, 1H), 4.47-4.40 (m, 1H), 4.23 (q, 1H), 3.64 (s, 3H), 2.28 (s, 3H), 2.25 (s, 6H), 1.84-1.73 (m, 1H), 1.71-1.62 (m, 1H), 1.33 (d, 3H), 0.89 (t, 3H).

### (2) (S)-2-({3′-[(R)-1-(4-Chloro-3-methyl-phenyl)-propylamino]-3,5-dimethyl-biphenyl-4-carbonyl}-amino)-propionic acid

To a solution of INT10 (320 mg, 0.649 mmol) in THF/water (4.5 mL, 2:1) was added LiOH hydrate (54.5 mg, 1.30 mmol) and the resulting mixture was stirred at room temperature for 3 hours. The reaction mixture was diluted with EtOAc and water. 1M HCl (1.30 mL) was then added and the organic layer was separated, washed with brine, dried over sodium sulfate, filtered and concentrated. The residue was purified by chromatography on silica gel (cyclohexane/EtOAc) to give Example EX6 as a white solid.

LC/MS Method 2: MS (ESI): 479 [M+H]⁺, rt=2.93 min. ¹H-NMR (DMSO-d₆): δ (ppm) 12.50 (br s, 1H), 8.61 (d, 1H), 7.37 (s, 1H), 7.32 (d, 1H), 7.22 (dd, 1H), 7.10 (s, 2H), 7.03 (t, 1H), 6.74 (s, 1H), 6.69 (d, 1H), 6.47 (d, 1H), 6.23 (d, 1H), 4.40-4.34 (m, 1H), 4.25 (q, 1H), 2.29 (s, 3H), 2.26 (s, 6H), 1.83-1.75 (m, 1H), 1.70-1.62 (m, 1H), 1.32 (d, 3H), 0.90 (t, 3H).

### Example EX7

### 1-({3′-[1-(4-Chloro-3-methyl-phenyl)-ethylamino]-3,5-dimethyl-biphenyl-4-carbonyl}-amino)-cyclopropanecarboxylic acid

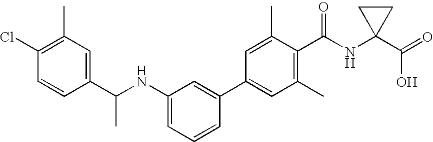

### (1) 1-[(3′-Amino-3,5-dimethyl-biphenyl-4-carbonyl)-amino]-cyclopropanecarboxylic acid ethyl ester, INT11

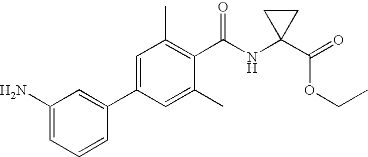

To a solution of INT1 (600 mg, 2.49 mmol) and 1-amino-cyclopropanecarboxylic acid ethyl ester hydrochloride (535 mg, 3.23 mmol) in DMF (12.5 mL) was added DIPEA (1.30 mL, 7.46 mmol) followed by TBTU (958 mg, 2.98 mmol) and the resulting mixture was stirred at room temperature overnight. The DMF was evaporated under reduced pressure and the residue dissolved in EtOAc. The organic layer was washed with 5% aqueous sodium bicarbonate, brine/water (1:1) and brine, dried over sodium sulfate, filtered and concentrated. The residue was purified by chromatography on silica gel (cyclohexane/EtOAc) to give INT11 as a white solid.

LC/MS method 2: MS (ESI): 353 [M+H]⁺, rt=1.90 min. ¹H-NMR (DMSO-d₆): δ (ppm) 8.91 (s, 1H), 7.20 (s, 2H), 7.07 (t, 1H), 6.80 (t, 1H), 6.73 (d, 1H), 6.54 (dd, 1H), 5.13 (s, 2H), 4.10 (q, 2H), 2.28 (s, 6H), 1.43 (q, 2H), 1.20 (t, 3H), 1.10 (q, 2H).

### (2) 1-({3′-[1-(4-Chloro-3-methyl-phenyl)-ethylamino]-3,5-dimethyl-biphenyl-4-carbonyl}-amino)-cyclopropanecarboxylic acid ethyl ester, INT12

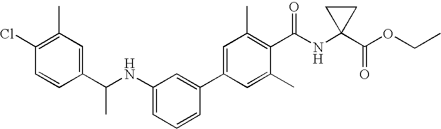

To a solution of INT11 (625 mg, 1.77 mmol) and INT4 (329 mg, 1.95 mmol) in MeOH (17 mL) was added decaborane (130 mg, 1.06 mmol) and the resulting mixture was stirred under argon overnight. The solvent was evaporated and the residue was purified by chromatography on silica gel (cyclohexane/EtOAc) to give INT12 as a white solid.

LC/MS method 2: MS (ESI): 505 [M+H]⁺, rt=3.29. ¹H-NMR (DMSO-d₆): δ (ppm) 8.85 (s, 1H), 7.36 (s, 1H), 7.30 (s, 1H), 7.20 (dd, 1H), 7.07 (s, 2H), 7.02 (t, 1H), 6.69 (d, 1H), 6.68 (d, 1H), 6.43 (dd, 1H), 6.23 (d, 1H), 4.51-4.44 (m, 1H), 4.09 (q, 2H), 2.29 (s, 3H), 2.26 (s, 6H), 1.42 (q, 2H), 1.40 (d, 3H), 1.20 (t, 3H), 1.10 (q, 2H).

### (3) 1-({3′-[1-(4-Chloro-3-methyl-phenyl)-ethylamino]-3,5-dimethyl-biphenyl-4-carbonyl}-amino)-cyclopropanecarboxylic acid

To a solution of INT12 (131 mg, 0.224 mmol) in THF/water (2 mL, 1:1) was added LiOH hydrate (37.6 mg, 0.894 mmol) and the resulting mixture was stirred at room temperature for 3 hours. The reaction mixture was diluted with EtOAc and water. 1M HCl (0.894 mL) was then added and the organic layer was separated, washed with brine, dried over sodium sulfate, filtered and concentrated. The residue was purified by chromatography on silica gel (cyclohexane/EtOAc) to give Example EX7 as a white solid.

LC/MS Method 2: MS (ESI): 477 [M+H]⁺, rt=2.89 min. ¹H-NMR (DMSO-d₆): δ (ppm) 12.35 (br s, 1H), 8.75 (s, 1H), 7.36 (s, 1H), 7.30 (s, 1H), 7.20 (dd, 1H), 7.05 (s, 2H), 7.01 (t, 1H), 6.68 (d, 1H), 6.68 (d, 1H), 6.43 (dd, 1H), 6.23 (d, 1H), 4.50-4.43 (m, 1H), 2.29 (s, 3H), 2.25 (s, 6H), 1.40 (d, 3H), 1.39 (q, 2H), 1.05 (q, 2H).

### Example EX8

### 1-({3′-[(R)-1-(4-Chloro-3-methyl-phenyl)-ethylamino]-3,5-dimethyl-biphenyl-4-carbonyl}-amino)-cyclopropanecarboxylic acid

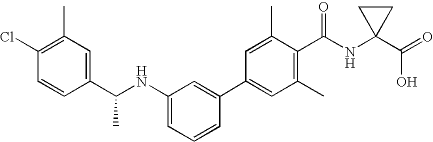

### (1) 1-({3′-[(R)-1-(4-Chloro-3-methyl-phenyl)-ethylamino]-3,5-dimethyl-biphenyl-4-carbonyl}-amino)-cyclopropanecarboxylic acid ethyl ester, INT13

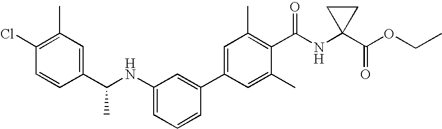

The title compound was obtained by preparative chiral separation of INT12 (Method A). Chiral HPLC method D: rt=12.68 min, heptane/EtOH (80:20).

LC/MS method 2: MS (ESI): 505 [M+H]⁺, rt=3.35 min. ¹H-NMR (DMSO-d₆): δ (ppm) 8.89 (s, 1H), 7.38 (s, 1H), 7.32 (s, 1H), 7.23 (dd, 1H), 7.09 (s, 2H), 7.04 (t, 1H), 6.71 (s, 1H), 6.70 (d, 1H), 6.45 (dd, 1H), 6.26 (d, 1H), 4.51-4.46 (m, 1H), 4.10 (q, 2H), 2.29 (s, 3H), 2.26 (s, 6H), 1.42 (q, 2H), 1.40 (d, 3H), 1.20 (t, 3H), 1.10 (q, 2H).

### (2) 1-({3′-[(R)-1-(4-Chloro-3-methyl-phenyl)-ethylamino]-3,5-dimethyl-biphenyl-4-carbonyl}-amino)-cyclopropanecarboxylic acid

To a solution of INT13 (397 mg, 0.786 mmol) in THF/water (8 mL, 1:1) was added LiOH hydrate (66.0 mg, 1.57 mmol) and the resulting mixture was stirred at room temperature for 2 hours. The reaction mixture was diluted with EtOAc and water. 1M HCl (1.57 mL) was then added and the organic layer was separated, washed with brine, dried over sodium sulfate, filtered and concentrated. The residue was purified by chromatography on silica gel (cyclohexane/EtOAc) to give Example EX8 as a white solid.

LC/MS Method 2: MS (ESI): 477 [M+H]⁺, rt=2.81 min. ¹H-NMR (DMSO-d₆): δ (ppm) 12.42 (br s, 1H), 8.78 (s, 1H), 7.38 (d, 1H), 7.32 (s, 1H), 7.22 (dd, 1H), 7.08 (s, 2H), 7.03 (t, 1H), 6.70 (d, 1H), 6.69 (d, 1H), 6.45 (dd, 1H), 6.26 (d, 1H), 4.51-4.45 (m, 1H), 2.29 (s, 3H), 2.25 (s, 6H), 1.40 (d, 3H), 1.39 (q, 2H), 1.05 (q, 2H).

### Example EX9

### (S)-2-({3′-[1-(4-Chloro-3-fluoro-phenyl)-ethylamino]-3,5-dimethyl-biphenyl-4-carbonyl}-amino)-propionic acid

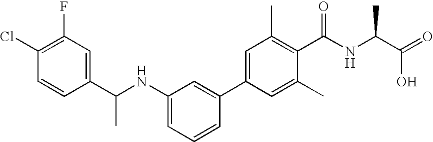

The title compound was prepared according to Scheme 1 following a procedure analogous to Example EX1 using the commercially available 1-(4-chloro-3-fluoro-phenyl)-ethanone in step 3.

LC/MS Method 2: MS (ESI): 469 [M+H]⁺, rt=2.67 min.

### Example EX10

### (S)-2-({3′-[(3,4-Dichlorophenyl)-ethylamino]-3,5-dimethyl-biphenyl-4-carbonyl}-amino)-propionic acid

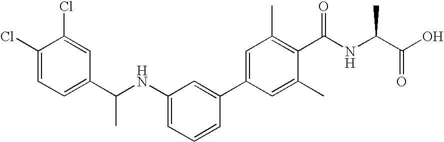

The title compound was prepared according to Scheme 1 following a procedure analogous to Example EX1 using the commercially available 1-(3,4-dichloro-phenyl)-ethanone in step 3.

LC/MS Method 2: MS (ESI): 485 [M+H]⁺, rt=3.18 min.

### Example EX11

### (S)-2-({3′-[1-(3,4-Dimethylphenyl)-ethylamino]-3,5-dimethyl-biphenyl-4-carbonyl}-amino)-propionic acid

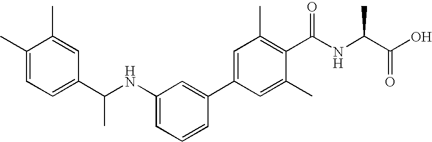

The title compound was prepared according to Scheme 1 following a procedure analogous to Example EX1 using the commercially available 1-(3,4-dimethyl-phenyl)-ethanone in step 3.

LC/MS Method 2: MS (ESI): 445 [M+H]⁺, rt=3.12 min.

### Example EX12

### (R)-2-({3′-[1-(4-Chloro-3-methyl-phenyl)-ethylamino]-3,5-dimethyl-biphenyl-4-carbonyl}-amino)-propionic acid

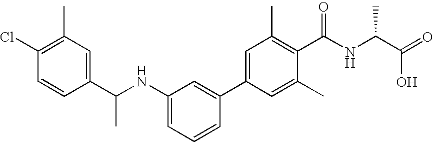

The title compound was prepared according to Scheme 1 following a procedure analogous to Example EX1 using D-alanine methyl ester hydrochloride in step 2 and INT4 in step 3.

LC/MS method 2: MS (ESI): 463 [M+H]⁺, rt=3.18 min.

### Example EX13

### (S)-2-({3′-[1-(4-Chloro-2,5-dimethyl-phenyl)-ethylamino]-3,5-dimethyl-biphenyl-4-carbonyl}-amino)-propionic acid

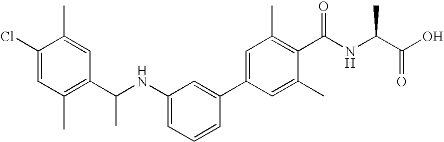

The title compound was prepared according to Scheme 1 following a procedure analogous to Example EX1 using INT15 (synthesis below) in step 3.

LC/MS Method 2: MS (ESI): 479 [M+H]⁺, rt=3.32 min.

### 1-(4-Chloro-2,5-dimethyl-phenyl)-ethanone, INT15

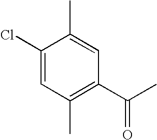

To a well stirred suspension of trichloroaluminum (11.38 g, 85.3 mmol) in carbon disulfide (60 mL) was added dropwise a solution of acetyl chloride (5.59 mL, 78.2 mmol) and 2-chloro-1,4-dimethyl-benzene (10.00 g, 71.1 mmol) in CS2 (82 mL) over 1 hour and the resulting mixture was stirred vigorously overnight. Water (100 mL) was added dropwise and the mixture was stirred for 1 hour at room temperature. Then, the organic layer was separated and the aqueous layer was further extracted with DCM. The combined organic layers were dried over sodium sulfate, filtered and concentrated. The residue was purified by chromatography on silica gel (cyclohexane/EtOAc) to give INT15 as a pale yellow solid.

LC/MS Method 1: MS (ESI): 183 [M+H]⁺, rt=1.35 min. ¹H-NMR (CDCl₃): δ (ppm) 7.55 (s, 1H), 7.22 (s, 1H), 2.55 (s, 3H), 2.47 (s, 3H), 2.38 (s, 3H).

### Example EX14

### (S)-2-({3′-[1-(4-Chloro-3-trifluoromethyl-phenyl)-ethylamino]-3,5-dimethyl-biphenyl-4-carbonyl}-methyl-amino)-propionic acid

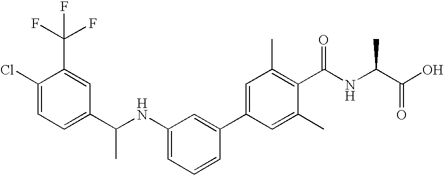

The title compound was prepared according to Scheme 1 following a procedure analogous to Example EX1 using the commercially available 1-(4-chloro-3-trifluoromethyl-phenyl)-ethanone in step 3.

LC/MS Method 2: MS (ESI): 519 [M+H]⁺, rt=3.21 min.

### Example EX15

### (S)-2-({3′-[1-(4-Fluoro-3-methoxy-phenyl)-ethylamino]-3,5-dimethyl-biphenyl-4-carbonyl}-amino)-propionic acid

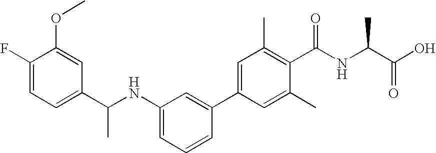

The title compound was prepared according to Scheme 1 following a procedure analogous to Example EX1 using the commercially available 1-(4-fluoro-3-methoxy-phenyl)-ethanone in step 3.

LC/MS Method 2: MS (ESI): 465 [M+H]⁺, rt=2.51 min.

### Example EX16

### (S)-2-({3′-[1-(4-Chloro-2-fluoro-5-methyl-phenyl)-ethylamino]-3,5-dimethyl-biphenyl-4-carbonyl}-amino)-propionic acid

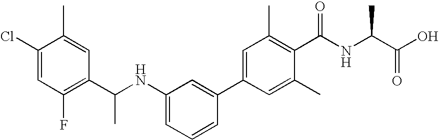

The title compound was prepared according to Scheme 1 following a procedure analogous to Example EX1 using the commercially available 1-(4-chloro-2-fluoro-5-methyl-phenyl)-ethanone in step 3.

LC/MS Method 2: MS (ESI): 483 [M+H]⁺, rt=2.94 min.

### Example EX17

### (S)-2-({3′-[1-(4-Fluoro-3-trifluoromethoxy-phenyl)-ethylamino]-3,5-dimethyl-biphenyl-4-carbonyl}-amino)-propionic acid

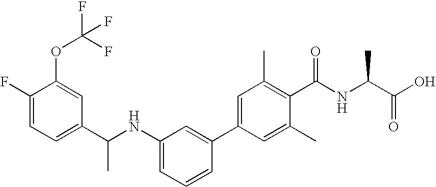

The title compound was prepared according to Scheme 1 following a procedure analogous to Example EX1 using the commercially available 1-(4-fluoro-3-trifluoromethoxy-phenyl)-ethanone in step 3.

LC/MS Method 2: MS (ESI): 519 [M+H]⁺, rt=2.84 min.

### Example EX18

### (S)-2-({3′-[1-(3-Fluoro-4-methoxy-phenyl)-ethylamino]-3,5-dimethyl-biphenyl-4-carbonyl}-amino)-propionic acid

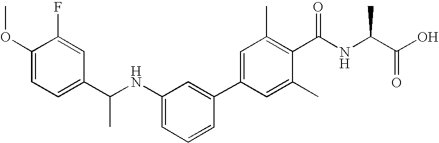

The title compound was prepared according to Scheme 1 following a procedure analogous to Example EX1 using the commercially available 1-(4-fluoro-3-trifluoromethoxy-phenyl)-ethanone in step 3.

LC/MS Method 2: MS (ESI): 465 [M+H]⁺, rt=2.51 min.

### Example EX19

### (S)-2-({3′-[1-(3-Fluoro-5-methyl-phenyl)-ethylamino]-3,5-dimethyl-biphenyl-4-carbonyl}-amino)-propionic acid

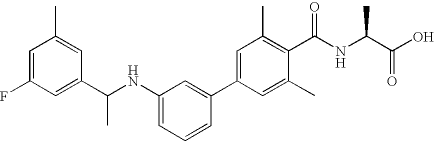

The title compound was prepared according to Scheme 1 following a procedure analogous to Example EX1 using the commercially available 1-(3-fluoro-5-methyl-phenyl)-ethanone in step 3.

LC/MS Method 2: MS (ESI): 449 [M+H]⁺, rt=2.68 min.

### Example EX20

### (S)-2-({3′-[1-(3,4-Dichloro-phenyl)-propylamino]-3,5-dimethyl-biphenyl-4-carbonyl}-amino)-propionic acid

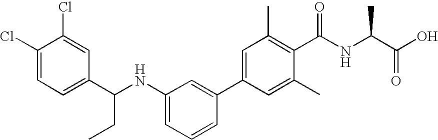

The title compound was prepared according to Scheme 1 following a procedure analogous to Example EX1 using the commercially available 1-(3,4-dichloro-phenyl)-propan-1-one in step 3.

LC/MS Method 2: MS (ESI): 499 [M+H]⁺, rt=3.34 min.

### Example EX21

### (S)-2-({3′-[1-(4-Chloro-3-methyl-phenyl)-ethylamino]-3,5-dimethyl-biphenyl-4-carbonyl}-amino)-3-hydroxy-propionic acid

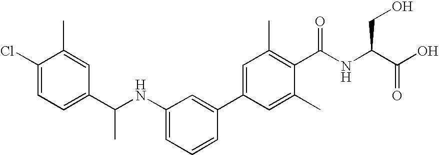

The title compound was prepared according to Scheme 1 following a procedure analogous to Example EX1 using serine methyl ester hydrochloride in step 2 and INT4 in step 3.

LC/MS Method 2: MS (ESI): 481 [M+H]⁺, rt=2.90 min.

### Example EX22

### (S)-2-({5′-[1-(4-Chloro-3-methyl-phenyl)-ethylamino]-2′-fluoro-3,5-dimethyl-biphenyl-4-carbonyl}-amino)-propionic acid

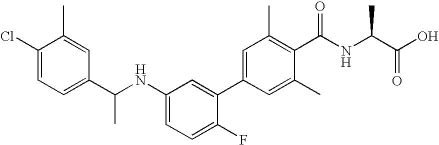

The title compound was prepared according to Scheme 1 following a procedure analogous to Example EX1 using (5-amino-2-fluoro-phenyl)-boronic acid in step 1 and INT4 in step 3.

LC/MS Method 2: MS (ESI): 483 [M+H]⁺, rt=3.21 min.

### Example EX23

### (S)-2-({5′-[(R)-1-(4-Chloro-3-methyl-phenyl)-ethylamino]-2′-fluoro-3,5-dimethyl-biphenyl-4-carbonyl}-amino)-propionic acid

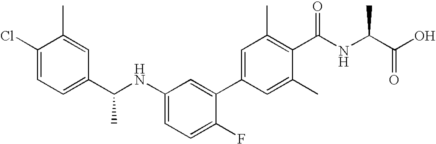

The title compound was prepared according to Scheme 1 following a procedure analogous to Example EX22 with an additional chiral separation step after step 3.

LC/MS Method 2: MS (ESI): 483 [M+H]⁺, rt=2.88 min.

### Example EX24

### (S)-2-({5′-[1-(4-Chloro-3-methyl-phenyl)-propylamino]-2′-fluoro-3,5-dimethyl-biphenyl-4-carbonyl}-amino)-propionic acid

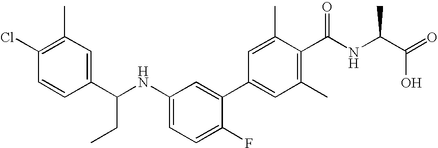

The title compound was prepared according to Scheme 1 following a procedure analogous to Example EX1 using (2-fluoro-5-amino-phenyl)-boronic acid in step 1 and INT8 in step 3.

LC/MS Method 2: MS (ESI): 497 [M+H]⁺, rt=3.06 min.

### Example EX25

### 1-({5′-[1-(4-Chloro-3-methyl-phenyl)-propylamino]-2′-fluoro-3,5-dimethyl-biphenyl-4-carbonyl}-amino)-cyclopropanecarboxylic acid

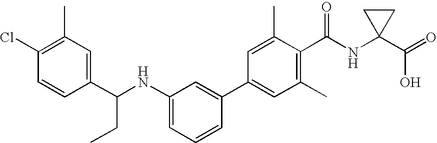

The title compound was prepared according to Scheme 1 following a procedure analogous to Example EX1 using (2-fluoro-5-amino-phenyl)-boronic acid in step 1, ethyl 1-amino-1-cyclopropanecarboxylate hydrochloride in step 2 and INT8 in step 3.

LC/MS Method 2: MS (ESI): 509 [M+H]⁺, rt=3.08 min.

### Example EX26

### 1-({5′-[1-(4-Chloro-3-methyl-phenyl)-ethylamino]-2′-fluoro-3,5-dimethyl-biphenyl-4-carbonyl}-amino)-cyclopropanecarboxylic acid

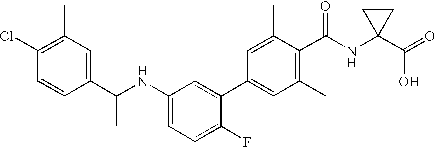

The title compound was prepared according to Scheme 1 following a procedure analogous to Example EX1 using (2-fluoro-5-amino-phenyl)-boronic acid in step 1, ethyl 1-amino-1-cyclopropanecarboxylate hydrochloride in step 2 and INT4 in step 3.

LC/MS Method 2: MS (ESI): 495 [M+H]⁺, rt=2.93 min.

### Example EX27

### 1-({3′-[1-(4-Chloro-3-methyl-phenyl)-propylamino]-3,5-dimethyl-biphenyl-4-carbonyl}-amino)-cyclopropanecarboxylic acid

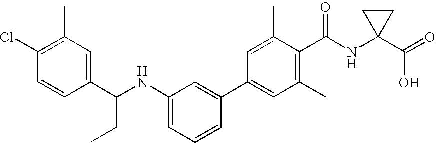

The title compound can be prepared according to Scheme 1 following a procedure analogous to Example EX1 using ethyl 1-amino-1-cyclopropanecarboxylate hydrochloride in step 2 and INT8 in step 3.

LC/MS Method 2: MS (ESI): 491 [M+H]⁺, rt=2.96 min.

### Example EX28

### (S)-2-({3′-[1-(3,5-Dimethyl-phenyl)-ethylamino]-3,5-dimethyl-biphenyl-4-carbonyl}-amino)-propionic acid

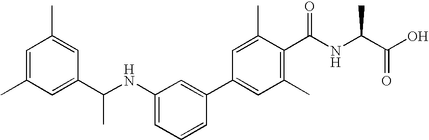

The title compound was prepared according to Scheme 1 following a procedure analogous to Example EX1 using 1-(3,5-dimethyl-phenyl)-ethanone in step 3.

LC/MS Method 2: MS (ESI): 445 [M+H]⁺, rt=2.74 min.

### Example EX29

### (S)-2-({3′-[1-(4-Chloro-3,5-dimethyl-phenyl)-ethylamino]-3,5-dimethyl-biphenyl-4-carbonyl}-amino)-propionic acid

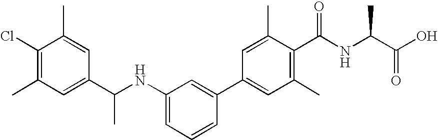

The title compound was prepared according to Scheme 1 following a procedure analogous to Example EX1 using 1-(4-chloro-3,5-dimethyl-phenyl)-ethanone in step 3.

LC/MS Method 2: MS (ESI): 479 [M+H]⁺, rt=2.93 min.

### Example EX30

### (S)-2-({3′-[1-(4-Methoxy-3-methyl-phenyl)-ethylamino]-3,5-dimethyl-biphenyl-4-carbonyl}-amino)-propionic acid

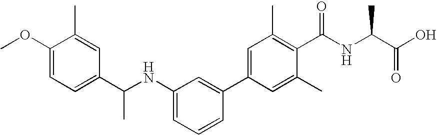

The title compound was prepared according to Scheme 1 following a procedure analogous to Example EX1 using 1-(4-methoxy-3-methyl-phenyl)-ethanone in step 3.

LC/MS Method 1: MS (ESI): 459 [M−H]⁻, rt=1.19 min.

### Example EX31

### (S)-2-({3′-[(4-Methoxy-phenyl)-ethylamino]-3,5-dimethyl-biphenyl-4-carbonyl}-amino)-propionic acid

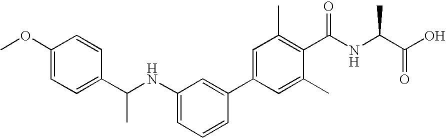

The title compound was prepared according to Scheme 1 following a procedure analogous to Example EX1 using 1-(4-methoxy-phenyl)-ethanone in step 3.

LC/MS Method 1: MS (ESI): 445 [M−H]⁻, rt=1.11 min.

### Example EX32

### (S)-2-({3′-[1-(3,4-Dimethoxy-phenyl)-ethylamino]-3,5-dimethyl-biphenyl-4-carbonyl}-amino)-propionic acid

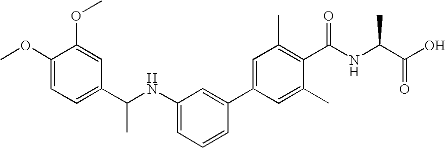

The title compound was prepared according to Scheme 1 following a procedure analogous to Example EX1 using 1-(3,4-dimethoxy-phenyl)-ethanone in step 3.

LC/MS Method 1: MS (ESI): 475 [M−H]⁻, rt=1.03 min.

### Example EX33

### (S)-2-({3′-[1-(4-Methoxy-3-trifluoromethyl-phenyl)-ethylamino]-3,5-dimethyl-biphenyl-4-carbonyl}-amino)-propionic acid

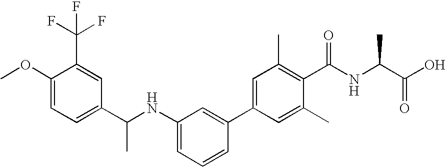

The title compound was prepared according to Scheme 1 following a procedure analogous to Example EX1 using 1-(4-methoxy-3-trifluoromethyl-phenyl)-ethanone in step 3.

LC/MS Method 1: MS (ESI): 513 [M−H]⁻, rt=1.33 min.

### Example EX34

### (S)-2-({3′-[1-(3,5-Difluoro-4-methoxy-phenyl)-ethylamino]-3,5-dimethyl-biphenyl-4-carbonyl}-amino)-propionic acid

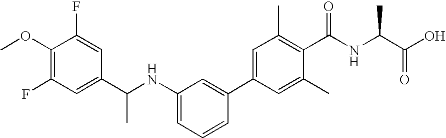

The title compound was prepared according to Scheme 1 following a procedure analogous to Example EX1 using 1-(3,5-difluoro-4-methoxy-phenyl)-ethanone in step 3.

LC/MS Method 1: MS (ESI): 481 [M−H]⁻, rt=1.32 min.

### Example EX35

### (S)-2-({3′-[1-(2-Fluoro-4-methoxy-phenyl)-ethylamino]-3,5-dimethyl-biphenyl-4-carbonyl}-amino)-propionic acid

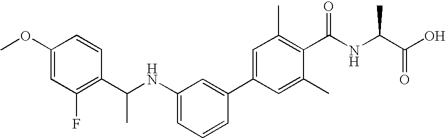

The title compound was prepared according to Scheme 1 following a procedure analogous to Example EX1 using 1-(2-fluoro-4-methoxy-phenyl)-ethanone in step 3.

LC/MS Method 1: MS (ESI): 463 [M−H]⁻, rt=1.27 min.

### Example EX36

### (S)-2-({3′-[(3-Chloro-5-fluoro-4-methoxy-phenyl)-ethylamino]-3,5-dimethyl-biphenyl-4-carbonyl}-amino)-propionic acid

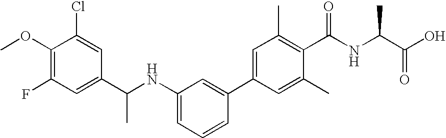

The title compound was prepared according to Scheme 1 following a procedure analogous to Example EX1 using 1-(3-chloro-5-fluoro-4-methoxy-phenyl)-ethanone in step 3.

LC/MS Method 1: MS (ESI): 497 [M−H]⁻, rt=1.38 min.

### Example EX37

### (S)-2-({3′-[1-(4-Methoxy-3,5-dimethyl-phenyl)-ethylamino]-3,5-dimethyl-biphenyl-4-carbonyl}-amino)-propionic acid

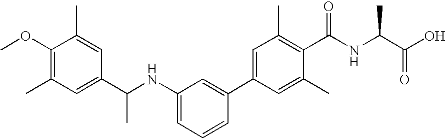

The title compound was prepared according to Scheme 1 following a procedure analogous to Example EX1 using 1-(4-methoxy-3,5-dimethyl-phenyl)-ethanone in step 3.

LC/MS Method 1: MS (ESI): 473 [M−H]⁻, rt=1.27 min.

### Example EX38

### (S)-2-({3′-[1-(4-Chloro-3-methyl-phenyl)-ethylamino]-3-methyl-biphenyl-4-carbonyl}-amino)-propionic acid

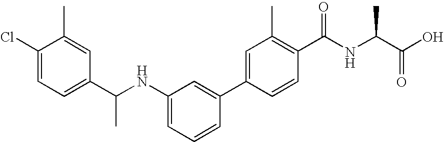

The title compound was prepared according to Scheme 1 following a procedure analogous to Example EX1 using 2-methyl-4-bromo-benzoic acid in step 1 and INT4 in step 3.

LC/MS Method 2: MS (ESI): 451 [M+H]⁺, rt=3.16 min.

### Example EX39

### (S)-2-({3′-[(S)-1-(4-Chloro-3-methyl-phenyl)-ethylamino]-3-methyl-biphenyl-4-carbonyl}-amino)-propionic acid

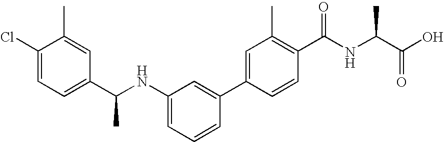

The title compound was prepared according to Scheme 1 following a procedure analogous to Example EX38 with an additional chiral separation (Method A) step after step 3:

LC/MS Method 2: MS (ESI): 451 [M+H]⁺, rt=3.16 min.

### Example EX40

### (S)-2-({3′-[(R)-1-(4-Chloro-3-methyl-phenyl)-ethylamino]-3-methyl-biphenyl-4-carbonyl}-amino)-propionic acid

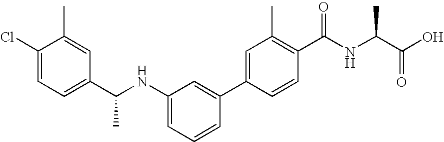

The title compound was prepared according to Scheme 1 following a procedure analogous to Example EX38 with an additional chiral separation (Method A) step after step 3:

LC/MS Method 2: MS (ESI): 451 [M+H]⁺, rt=3.16 min.

### Example EX41

### (S)-2-({3′-[(4-Chloro-2,5-dimethyl-phenyl)-ethylamino]-3-methyl-biphenyl-4-carbonyl}-amino)-propionic acid

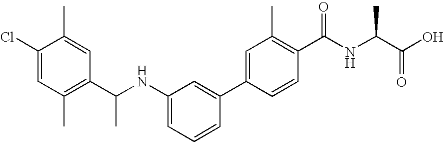

The title compound was prepared according to Scheme 1 following a procedure analogous to Example EX1 using 2-methyl-4-bromo-benzoic acid in step 1 and INT15 in step 3.

LC/MS Method 2: MS (ESI): 465 [M+H]⁺, rt=3.28 min.

### Example EX42

### (S)-2-({3′-[1-(4-Chloro-3-methyl-phenyl)-ethylamino]-3-ethyl-biphenyl-4-carbonyl}-amino)-propionic acid

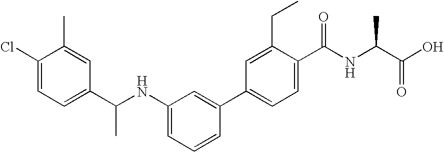

The title compound was prepared according to Scheme 1 following a procedure analogous to Example EX1 using 4-bromo-2-ethyl-benzoic acid in step 1 and INT4 in step 3.

LC/MS Method 2: MS (ESI): 465 [M+H]⁺, rt=2.81 min.

### Example EX43

### (S)-2-({3′-[1-(4-Chloro-3-methyl-phenyl)-ethylamino]-3-ethyl-biphenyl-4-carbonyl}-methyl-amino)-propionic acid

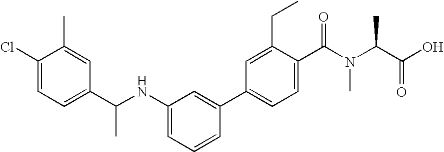

The title compound was prepared according to Scheme 1 following a procedure analogous to Example EX1 using 4-bromo-2-ethyl-benzoic acid in step 1, N-methyl alanine methyl ester hydrochloride in step 2 and INT4 in step 3.

LC/MS Method 2: MS (ESI): 479 [M+H]⁺, rt=2.91 min.

### Example EX44

### (S)-2-({3′-[1-(4-Chloro-3,5-dimethyl-phenyl)-ethylamino]-3-ethyl-biphenyl-4-carbonyl}-amino)-propionic acid

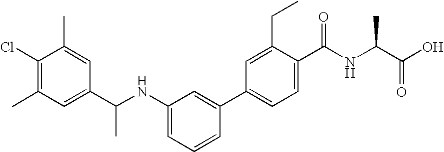

The title compound was prepared according to Scheme 1 following a procedure analogous to Example EX1 using 4-bromo-2-ethyl-benzoic acid in step 1 and 1-(4-chloro-3,5-dimethyl-phenyl)-ethanone in step 3.

LC/MS Method 2: MS (ESI): 479 [M+H]⁺, rt=2.95 min.

### Example EX45

### (S)-2-({3′-[1-(4-Chloro-3,5-dimethyl-phenyl)-ethylamino]-3-ethyl-biphenyl-4-carbonyl}-methyl-amino)-propionic acid

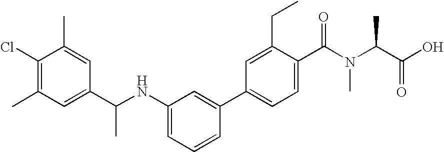

The title compound was prepared according to Scheme 1 following a procedure analogous to Example EX1 using 4-bromo-2-ethyl-benzoic acid in step 1, N-methyl alanine methyl ester hydrochloride in step 2 and 1-(4-chloro-3,5-dimethyl-phenyl)-ethanone in step 3.

LC/MS Method 2: MS (ESI): 493 [M+H]⁺, rt=3.04 min.

### Example EX46

### (S)-2-({3-Chloro-3′-[1-(4-chloro-3-methyl-phenyl)-ethylamino]-biphenyl-4-carbonyl}-amino)-propionic acid

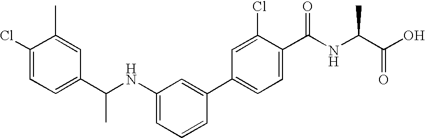

The title compound was prepared according to Scheme 1 following a procedure analogous to Example EX1 using 4-bromo-2-chloro-benzoic acid in step 1 and INT4 in step 3.

LC/MS Method 2: MS (ESI): 471 [M+H]⁺, rt=2.77 min.

### Example EX47

### (S)-2-({3-Chloro-3′-[1-(4-chloro-3-methyl-phenyl)-ethylamino]-biphenyl-4-carbonyl}-methyl-amino)-propionic acid

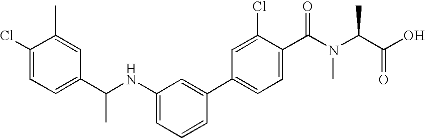

The title compound was prepared according to Scheme 1 following a procedure analogous to Example EX1 using 4-bromo-2-chloro-benzoic acid in step 1, N-methyl alanine methyl ester hydrochloride in step 2 and INT4 in step 3.

LC/MS Method 2: MS (ESD: 485 [M+H]⁺, rt=2.84 min.

### Example EX48

### (S)-2-({3′-[1-(4-Chloro-3-methyl-phenyl)-ethylamino]-3-fluoro-biphenyl-4-carbonyl}-amino)-propionic acid

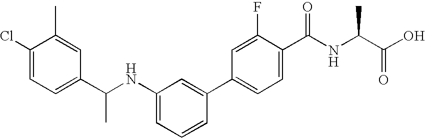

The title compound was prepared according to Scheme 1 following a procedure analogous to Example EX1 using 4-bromo-2-fluoro-benzoic acid in step 1 and INT4 in step 3.

LC/MS Method 2: MS (ESI): 455 [M+H]⁺, rt=2.78 min.

### Example EX49

### (S)-2-({3′-[1-(4-Chloro-3-methyl-phenyl)-ethylamino]-3-fluoro-biphenyl-4-carbonyl}-methyl-amino)-propionic acid

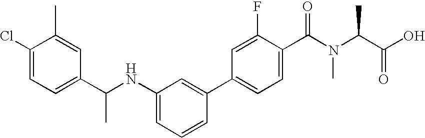

The title compound was prepared according to Scheme 1 following a procedure analogous to Example EX1 using 4-bromo-2-fluoro-benzoic acid in step 1, N-methyl alanine methyl ester hydrochloride in step 2 and INT4 in step 3.

LC/MS Method 2: MS (ESI): 469 [M+H]⁺, rt=2.76 min.

### Example EX50

### (S)-2-({3′-[1-(4-Chloro-3-methyl-phenyl)-ethylamino]-3,5-difluoro-biphenyl-4-carbonyl}-amino)-propionic acid

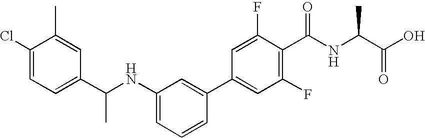

The title compound was prepared according to Scheme 1 following a procedure analogous to Example EX1 using 4-bromo-2,6-difluoro-benzoic acid in step 1 and INT4 in step 3.

LC/MS Method 2: MS (ESI): 473 [M+H]⁺, rt=2.74 min.

### Example EX51

### (S)-2-({3′-[1-(4-Chloro-3-methyl-phenyl)-ethylamino]-3,5-difluoro-biphenyl-4-carbonyl}-methyl-amino)-propionic acid

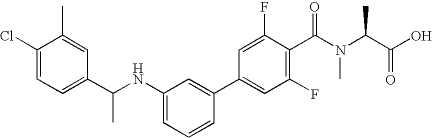

The title compound was prepared according to Scheme 1 following a procedure analogous to Example EX1 using 4-bromo-2,6-difluoro-benzoic acid in step 1, N-methyl alanine methyl ester hydrochloride in step 2 and INT4 in step 3.

LC/MS Method 2: MS (ESI): 487 [M+H]⁺, rt=2.83 min.

### Example EX52

### (S)-2-({3-Chloro-3′-[1-(4-chloro-3,5-dimethyl-phenyl)-ethylamino]-biphenyl-4-carbonyl}-amino)-propionic acid

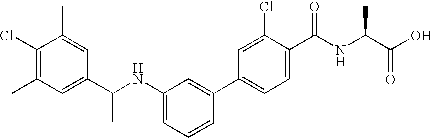

The title compound was prepared according to Scheme 1 following a procedure analogous to Example EX1 using 4-bromo-2-chloro-benzoic acid in step 1 and 1-(4-chloro-3,5-dimethyl-phenyl)-ethanone in step 3.

LC/MS Method 2: MS (ESI): 519 [M+H]⁺, rt=2.95 min.

### Example EX53

### (S)-2-({3-Chloro-3′-[1-(4-chloro-3,5-dimethyl-phenyl)-ethylamino]-biphenyl-4-carbonyl}-methyl-amino)-propionic acid

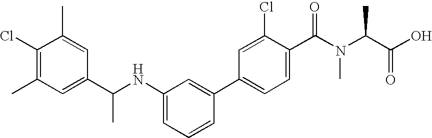

The title compound was prepared according to Scheme 1 following a procedure analogous to Example EX1 using 4-bromo-2-chloro-benzoic acid in step 1, N-methyl alanine methyl ester hydrochloride in step 2 and 1-(4-chloro-3,5-dimethyl-phenyl)-ethanone in step 3.

LC/MS Method 2: MS (ESI): 499 [M+H]⁺, rt=2.97 min.

### Example EX54

### (S)-2-({3′-[1-(4-Chloro-3,5-dimethyl-phenyl)-ethylamino]-3-fluoro-biphenyl-4-carbonyl}-amino)-propionic acid

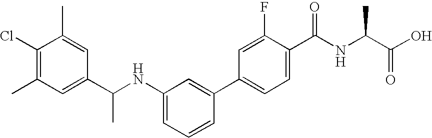

The title compound was prepared according to Scheme 1 following a procedure analogous to Example EX1 using 4-bromo-2-fluoro-benzoic acid in step 1 and 1-(4-chloro-3,5-dimethyl-phenyl)-ethanone in step 3.

LC/MS Method 2: MS (ESI): 469 [M+H]⁺, rt=2.90 min.

### Example EX55

### (S)-2-({3′-[1-(4-Chloro-3,5-dimethyl-phenyl)-ethylamino]-3-fluoro-biphenyl-4-carbonyl}-methyl-amino)-propionic acid

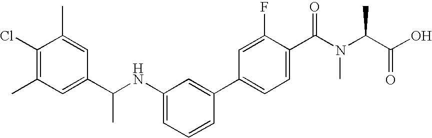

The title compound was prepared according to Scheme 1 following a procedure analogous to Example EX1 using 4-bromo-2-fluoro-benzoic acid in step 1, N-methyl alanine methyl ester hydrochloride in step 2 and 1-(4-chloro-3,5-dimethyl-phenyl)-ethanone in step 3.

LC/MS Method 2: MS (ESI): 483 [M+H]⁺, rt=2.90 min.

### Example EX56

### (S)-2-({3′-[1-(4-Chloro-3,5-dimethyl-phenyl)-ethylamino]-3,5-difluoro-biphenyl-4-carbonyl}-amino)-propionic acid

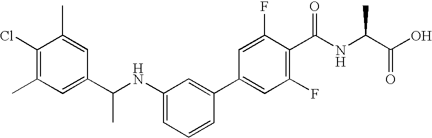

The title compound was prepared according to Scheme 1 following a procedure analogous to Example EX1 using 4-bromo-2,6-difluoro-benzoic acid in step 1 and 1-(4-chloro-3,5-dimethyl-phenyl)-ethanone in step 3.

LC/MS Method 2: MS (ESI): 487 [M+H]⁺, rt=2.85 min.

### Example EX57

### (S)-2-({3′-[1-(4-Chloro-3,5-dimethyl-phenyl)-ethylamino]-3,5-difluoro-biphenyl-4-carbonyl}-methyl-amino)-propionic acid

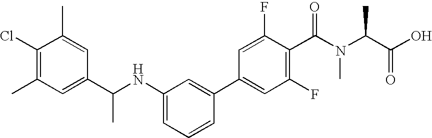

The title compound was prepared according to Scheme 1 following a procedure analogous to Example EX1 using 4-bromo-2,6-difluoro-benzoic acid in step 1, N-methyl alanine methyl ester hydrochloride in step 2 and 1-(4-chloro-3,5-dimethyl-phenyl)-ethanone in step 3.

LC/MS Method 2: MS (ESI): 501 [M+H]⁺, rt=2.97 min.

### Example EX58

### (S)-2-({3′-[1-(4-Chloro-3-methyl-phenyl)-propylamino]-3-methyl-biphenyl-4-carbonyl}-methyl-amino)-propionic acid

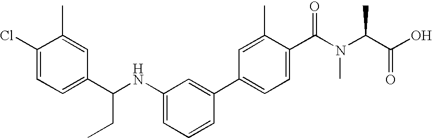

The title compound was prepared according to Scheme 1 following a procedure analogous to Example EX1 using 4-bromo-2-methyl-benzoic acid in step 1, N-methyl alanine methyl ester hydrochloride in step 2 and INT8 in step 3.

LC/MS Method 2: MS (ESI): 479 [M+H]⁺, rt=2.93 min.

### Example EX59

### (S)-2-({3′-[1-(4-Chloro-3,5-dimethyl-phenyl)-ethylamino]-3-methyl-biphenyl-4-carbonyl}-methyl-amino)-propionic acid

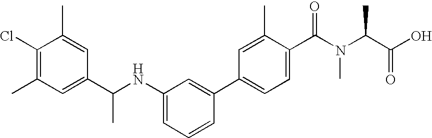

The title compound was prepared according to Scheme 1 following a procedure analogous to Example EX1 using 4-bromo-2-methyl-benzoic acid in step 1, N-methyl alanine methyl ester hydrochloride in step 2 and 1-(4-chloro-3,5-dimethyl-phenyl)-ethanone in step 3.

LC/MS Method 2: MS (ESI): 479 [M+H]⁺, rt=2.96 min.

### Example EX60

### (S)-2-({3-Chloro-3′-[1-(4-chloro-3-methyl-phenyl)-propylamino]-biphenyl-4-carbonyl}-amino)-propionic acid

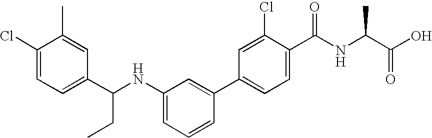

The title compound was prepared according to Scheme 1 following a procedure analogous to Example EX1 using 4-bromo-2-chloro-benzoic acid in step 1 and INT8 in step 3.

LC/MS Method 2: MS (ESI): 485 [M+H]⁺, rt=3.06 min.

### Example EX61

### (S)-2-({3-Chloro-3′-[1-(4-chloro-3-methyl-phenyl)-propylamino]-biphenyl-4-carbonyl}-methyl-amino)-propionic acid

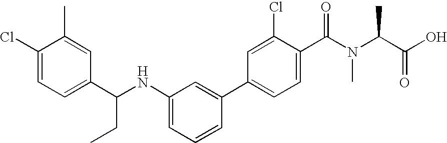

The title compound was prepared according to Scheme 1 following a procedure analogous to Example EX1 using 4-bromo-2-chloro-benzoic acid in step 1, N-methyl alanine methyl ester hydrochloride in step 2 and INT8 in step 3.

LC/MS Method 2: MS (ESI): 499 [M+H]⁺, rt=3.17 min.

### Example EX62

### (S)-2-({3′-[1-(4-Chloro-3-methyl-phenyl)-ethylamino]-3-trifluoromethyl-biphenyl-4-carbonyl}-amino)-propionic acid

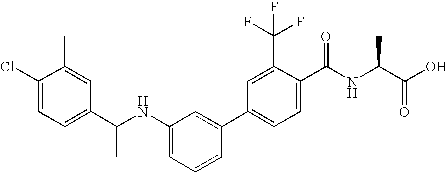

### (1) 3′-Amino-3-trifluoromethyl-biphenyl-4-carboxylic acid methyl ester, INT16

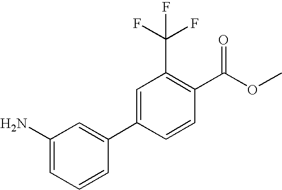

A flask was charged with PEPPSI-IPr® (43 mg, 0.063 mmol), potassium carbonate (2.172 g, 15.72 mmol), (3-aminophenyl)-boronic acid (1.169 g, 7.54 mmol) and 4-chloro-2-trifluoromethyl-benzoic acid methyl ester (1.5 g, 6.29 mmol) and was purged with nitrogen. Dioxane (17 mL) was added via syringe and the mixture was stirred at 60° C. over the weekend. The mixture was diluted with ethyl acetate (100 mL) and washed with water and brine. After drying over sodium sulfate, filtration and evaporation the crude was purified by chromatography on silica gel using cyclohexane and ethyl acetate (from 10% to 20%).

LC/MS Method 1: MS (ESI): 336.9 [M+H]⁺, rt=1.07 min. ¹H-NMR (CDCl₃): δ (ppm) 7.92 (br s, 1H), 7.86 (d, 1H), 7.77 (d, 1H), 7.26 (t, 1H), 6.98 (d, 1H), 6.9 (br s, 1H), 6.75 (d, 1H), 3.95 (s, 3H), 3.8 (v br s, 2H), 1.54 (s, 6H).

### (2) 3′-Amino-3-trifluoromethyl-biphenyl-4-carboxylic acid, INT17

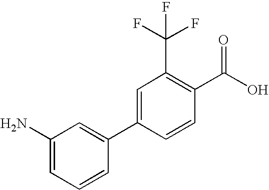

A solution of intermediate INT16 (620 mg, 2.1 mmol) in 50 mL of THF was treated with an aqueous 1M LiOH solution (8.4 mL, 8.4 mmol). The mixture was stirred overnight at 60° C. Most of the THF was then evaporated and water was added. The pH was adjusted to about 2-3 with 2N HCl until a white precipitate was observed. This mixture was extracted twice with ethyl acetate. The organic layers were combined, dried over sodium sulfate, filtered and evaporated to give an off-white powder.

LC/MS Method 1: MS (ESI): 322.9 [M+H]⁺, rt=0.73 min. ¹H-NMR (DMSO-d₆): δ (ppm) 7.91 (m, 3H), 7.16 (t, 1H), 6.93 (br s, 1H), 6.87 (d, 1H), 6.65 (d, 1H), 3.33 (br s, 2H).

### (3) (S)-2-[(3′-Amino-3-trifluoromethyl-biphenyl-4-carbonyl)-amino]-propionic acid methyl ester, INT18

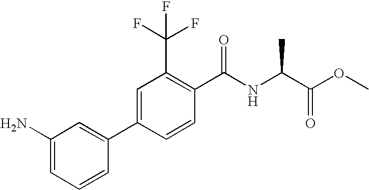

A solution of acid INT17 (230 mg, 0.818 mmol), alanine methyl ester hydrochloride (171 mg, 1.227 mmol), DIEA (0.457 ml, 2.62 mmol) and TBTU (341 mg, 1.063 mmol) in DMF (20 mL) was stirred at room temperature overnight. The DMF was removed under high vacuum. The residue was dissolved in ethyl acetate (25 mL) and washed with 1M sodium bicarbonate and brine. The organic layer was dried over sodium sulfate, filtered and evaporated. The crude was purified by chromatography on silica gel using cyclohexane and ethyl acetate (20%).

LC/MS Method 1: MS (ESI): 367.02 [M+H]⁺, rt=0.81 min.

UPLC: rt=1.16 min.

### (4) (S)-2-({3′-[1-(4-Chloro-3-methyl-phenyl)-ethylamino]-3-trifluoromethyl-biphenyl-4-carbonyl}-amino)-propionic acid methyl ester, INT19

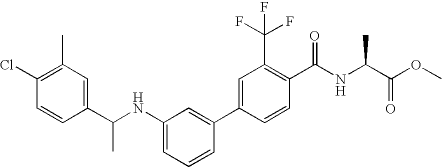

A solution of aniline INT18 (100 mg, 0.273 mmol), 1-(4-Chloro-3-methyl-phenyl)-ethanone (59.8 mg, 0.355 mmol) and decaborane (16.68 mg, 0.136 mmol) in 2 mL of MeOH was stirred overnight. Another 10 mg portion of decaborane was added and stirring was continued for 3 hours. The solvent was then evaporated and the crude was purified by chromatography on silica gel using cyclohexane and ethyl acetate (from 10% to 25%).

LC/MS Method 2: MS (ESI): 519 [M+H]⁺, rt=3.12 min. ¹H-NMR (DMSO-d₆): δ (ppm) 8.96 (d, 1H), 7.85 (d, 1H), 7.73 (s, 1H), 7.56 (d, 1H), 7.33 (s, 1H), 7.32 (d, 1H), 7.23 (d, 1H), 7.11 (t, 1H), 6.82 (br m, 2H), 6.53 (d, 1H), 6.39 (d, 1H), 4.53 (m, 1H), 4.45 (m, 1H), 3.67 (s, 3H), 2.29 (s, 3H), 1.41 (d, 3H), 1.34 (d, 3H).

### (5) (S)-2-({3′-[1-(4-Chloro-3-methyl-phenyl)-ethylamino]-3-trifluoromethyl-biphenyl-4-carbonyl}-amino)-propionic acid

A solution of ester INT19 (110 mg, 0.212 mmol) in 9 mL of THF was treated with a 1M LiOH solution (0.85 mL, 0.85 mmol) and stirred vigorously overnight. Then, 2M HCl (0.4 mL) was added and most of the THF was evaporated. The residue was diluted with water, the pH was adjusted to 4 with a few drops of 0.2M HCl upon which a white precipitate was formed. The mixture was extracted twice with ethyl acetate. The organic extracts were dried over sodium sulfate, filtered and evaporated to give the title compound Example EX62 as a white foam.

LC/MS Method 2: MS (ESI): 505 [M+H]⁺, rt=2.81 min. ¹H-NMR (DMSO-d₆): δ (ppm) 12.63 (v br s, 1H), 8.74 (br s, 1H), 7.85 (d, 1H), 7.72 (s, 1H), 7.56 (d, 1H), 7.38 (s, 1H), 7.32 (d, 1H), 7.23 (d, 1H), 7.11 (t, 1H), 6.81 (m, 2H), 6.53 (d, 1H), 6.4 (d, 1H), 4.53 (m, 1H), 4.33 (m, 1H), 2.29 (s, 3H), 1.41 (d, 3H), 1.33 (d, 3H).

### Example EX63

### (S)-2-({3′-[1-(4-Chloro-3-methyl-phenyl)-ethylamino]-3-trifluoromethyl-biphenyl-4-carbonyl}-methyl-amino)-propionic acid

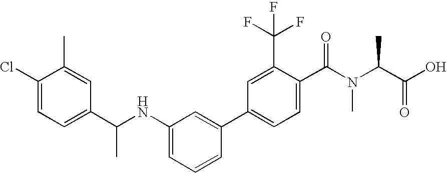

### (1) (S)-2-[(3′-Amino-3-trifluoromethyl-biphenyl-4-carbonyl)-methyl-amino]-propionic acid methyl ester, INT20

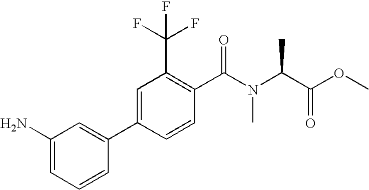

A solution of acid INT17 (160 mg, 0.569 mmol), N-methyl alanine methyl ester hydrochloride (131 mg, 0.853 mmol), DIPEA (0.298 ml, 1.707 mmol) and TBTU (237 mg, 0.74 mmol) in DMF (1 mL) was stirred at room temperature overnight. The DMF was removed under high vacuum. The residue was dissolved in ethyl acetate (25 mL) and washed with 1M sodium bicarbonate and brine. The organic layer was dried over sodium sulfate, filtered and evaporated. The crude was purified by chromatography on silica gel using cyclohexane and ethyl acetate (from 10% to 50%).

LC/MS Method 1: MS (ESI): 381.03 [M+H]⁺, rt=0.93 min.

UPLC: rt=1.39 min.

### (2) (S)-2-({3′-[1-(4-Chloro-3-methyl-phenyl)-ethylamino]-3-trifluoromethyl-biphenyl-4-carbonyl}-methyl-amino)-propionic acid methyl ester, INT21

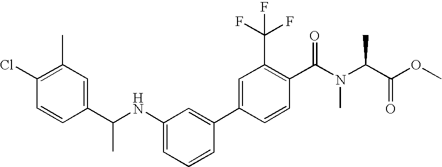

A solution of aniline INT20 (50 mg, 0.131 mmol), 1-(4-chloro-3-methylphenyl)ethanone (26.6 mg, 0.158 mmol) and decaborane (11.3 mg, 0.092 mmol) in 1 mL of MeOH was stirred over the weekend. The solvent was then evaporated and the crude was purified by chromatography on silica gel using cyclohexane and ethyl acetate (from 5% to 15%).

LC/MS Method 2: MS (ESD: 533 [M+H]⁺, rt=3.26 min.

¹H-NMR (DMSO-d₆): high temperature (120° C.) δ (ppm) 7.84 (d, 1H), 7.75 (s, 1H), 7.39 (d, 1H), 7.36 (s, 1H), 7.27 (d, 1H), 7.22 (d, 1H), 7.12 (t, 1H), 6.87 (s, 1H), 6.84 (d, 1H), 6.6 (d, 1H), 5.8 (d, 1H), 5.04 (v br m, 1H), 4.56 (m, 1H), 3.71 (s, 3H), 2.74 (br s, 3H), 2.32 (s, 3H), 1.45 (m, 6H).

### (3) (S)-2-({3′-[1-(4-Chloro-3-methyl-phenyl)-ethylamino]-3-trifluoromethyl-biphenyl-4-carbonyl}-methyl-amino)-propionic acid

A solution of ester INT21 (50 mg, 0.094 mmol) in 5 mL of THF was treated with a 1M LiOH solution (0.38 mL, 0.38 mmol) and stirred vigorously overnight. Then, 2M HCl (0.2 mL) was added and most of the THF was evaporated. The residue was diluted with water and the pH was adjusted to 4 with a few drops of 0.2M HCl upon which a white precipitate was formed. The mixture was extracted twice with ethyl acetate. The organic extracts were dried over sodium sulfate, filtered and evaporated to give the title compound Example EX63 as a white foam.

LC/MS Method 2: MS (ESI): 519 [M+H]⁺, rt=2.93 min. UPLC: rt=2.23 min.

¹H-NMR (DMSO-d₆): high temperature (120° C.) δ (ppm) 7.81 (br d, 1H), 7.74 (s, 1H), 7.4 (d, 1H), 7.36 (s, 1H), 7.28 (d, 1H), 7.22 (d, 1H), 7.11 (t, 1H), 6.87 (m, 1H), 6.85 (d, 1H), 6.58 (d, 1H), 5.8 (br m, 1H), 5.03 (v br m, 1H), 4.55 (br m, 1H), 2.32 (s, 3H), 1.48 (d, 3H), 1.38 (v br m, 3H). Signal of N—CH₃ group not visible due to overlap with water. Signal of carboxylic acid not observed.

### Example EX64

### (S)-2-({3′-[1-(4-Chloro-3,5-dimethyl-phenyl)-ethylamino]-3-trifluoromethyl-biphenyl-4-carbonyl}-amino)-propionic acid

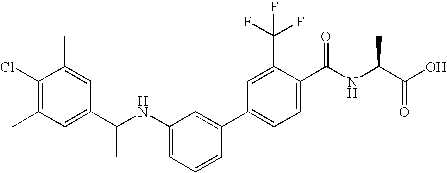

The title compound was prepared according to Scheme 1 following a procedure analogous to Example EX1 using intermediate INT17 in step 2 and 1-(4-chloro-3,5-dimethyl-phenyl)-ethanone in step 3.

LC/MS Method 2: MS (ESI): 519 [M+H]⁺, rt=2.95 min.

### Example EX65

### (S)-2-({3′-[1-(4-Chloro-3,5-dimethyl-phenyl)-ethylamino]-3-trifluoromethyl-biphenyl-4-carbonyl}-methyl-amino)-propionic acid

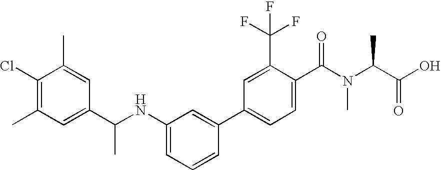

The title compound was prepared according to Scheme 1 following a procedure analogous to Example EX1 using intermediate INT17, N-methyl alanine methyl ester hydrochloride in step 2 and 1-(4-chloro-3,5-dimethyl-phenyl)-ethanone in step 3.

LC/MS Method 2: MS (ESI): 533 [M+H]⁺, rt=3.06 min.

### Example EX66

### (S)-2-({3′-[1-(4-Chloro-3-methyl-phenyl)-propylamino]-3-trifluoromethyl-biphenyl-4-carbonyl}-amino)-propionic acid

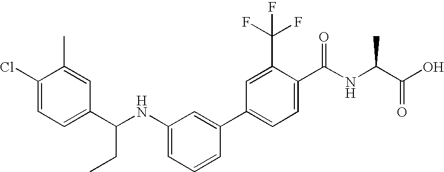

The title compound was prepared according to Scheme 1 following a procedure analogous to Example EX1 using intermediate INT17 in step 2 and INT8 in step 3.

LC/MS Method 2: MS (ESI): 519 [M+H]⁺, rt=3.14 min.

### Example EX67

### (S)-2-({3′-[1-(4-Chloro-3-methyl-phenyl)-propylamino]-3-trifluoromethyl-biphenyl-4-carbonyl}-methyl-amino)-propionic acid

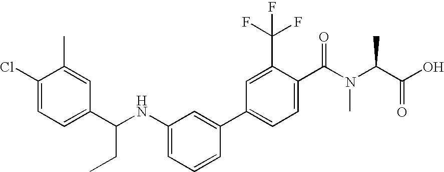

The title compound was prepared according to Scheme 1 following a procedure analogous to Example EX1 using intermediate INT17 and N-methyl alanine methyl ester hydrochloride in step 2 and INT8 in step 3.

LC/MS Method 2: MS (ESI): 533 [M+H]⁺, rt=3.29 min.

Alternatively, agents of the invention may be prepared by a reaction sequence involving reductive amination of an aldehyde or ketone with an aniline, Suzuki-type coupling with an appropriate boronic acid or ester, coupling with an appropriate amino ester, and saponification of the ester followed by an optional deprotection step as shown in Scheme 2 below:

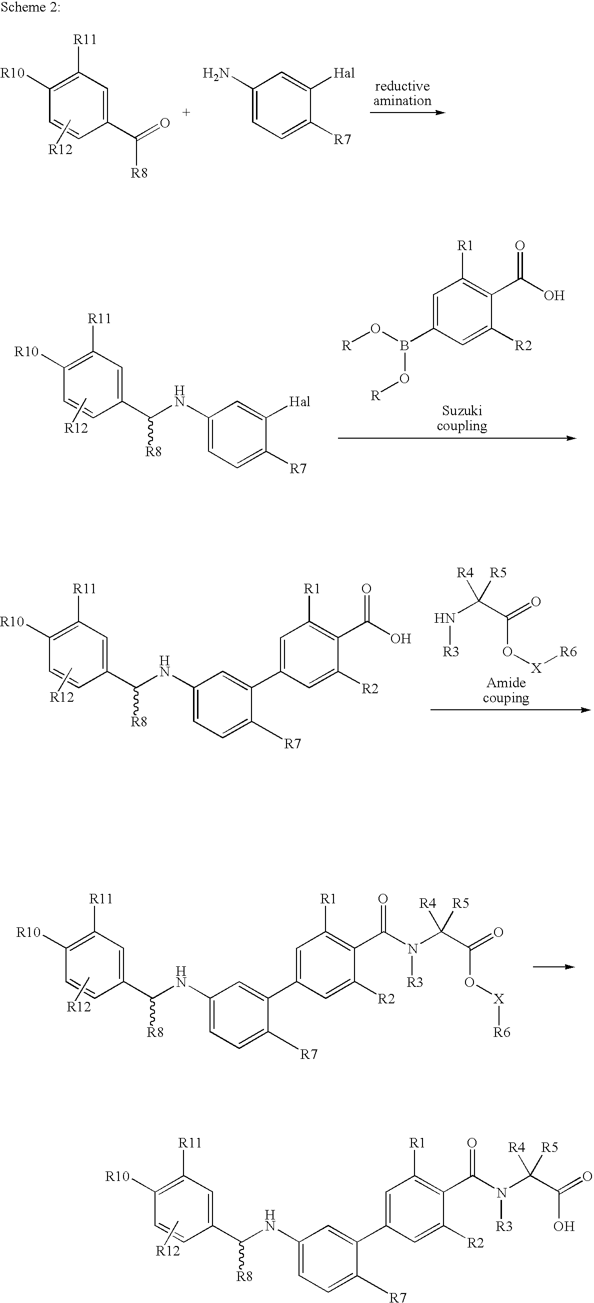

### Example EX68

### ({3′-[1-(4-Chloro-3-methyl-phenyl)-ethylamino]-3,5-dimethyl-biphenyl-4-carbonyl}-amino)-acetic acid

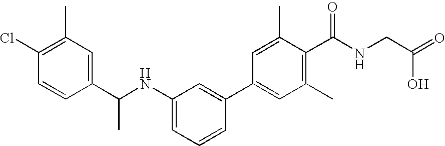

### (1) (3-Bromo-phenyl)-[1-(4-chloro-3-methyl-phenyl)-ethyl]-amine, INT22

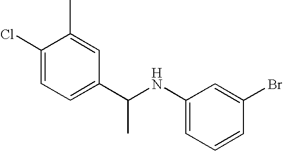

To a solution of ketone INT4 (10 g, 59.4 mmol) and 3-bromoaniline (9.58 g, 54.0 mmol) in MeOH (540 mL) was added decaborane and the resulting mixture was stirred at room temperature overnight. The mixture was concentrated and taken up in ether and cooled to 0° C. HCl (4M in dioxane, 30 mL) was added and the resulting white precipitate was collected by filtration and washed with ether. The white solid was suspended in DCM and after washing with saturated aqueous sodium bicarbonate, the organic layer was separated, dried over sodium sulfate, filtered and concentrated to give INT22 as a colorless oil.

LC/MS method 2: (ESI): 324-328 [M+H]⁺, rt=3.79 min. ¹H-NMR (DMSO-d₆): δ (ppm) 7.32-7.29 (m, 2H), 7.16 (dd, 1H), 6.89 (t, 1H), 6.62 (t, 1H), 6.56 (dd, 1H), 6.45 (d, 1H), 6.42 (dd, 1H), 4.45-4.38 (m, 1H), 2.86 (s, 2H), 1.36 (d, 3H).

### (2) 2,6-Dimethyl-4-(4,4,5,5-tetramethyl-[1,3,2]dioxaborolan-2-yl)-benzoic acid, INT23

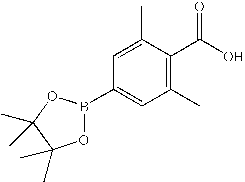

Argon was bubbled for 15 minutes through a mixture of bis(pinacoloto)diboron (1.55 g, 6.11 mmol), 4-bromo-2,5-dimethylbenzoic acid (1 g, 4.37 mmol) and potassium acetate (2.57 g, 26.2 mmol) in dioxane (44 mL). Then PdCl₂(dppf) (0.16 g, 0.218 mmol) was added and the resulting mixture was stirred at 80° C. overnight. After cooling down, the mixture was concentrated and partitioned between ether and 5% aqueous sodium bicarbonate. The organic layer was further extracted with 5% aqueous sodium bicarbonate and the combined aqueous layers were cooled to 0° C. and acidified to pH=2 with 2M HCl. The mixture was then extracted three times with EtOAc and the combined organic layers were washed with brine, dried over sodium sulfate, filtered and concentrated. The residue was triturated in cyclohexane and collected by filtration to give INT23 as a brown solid.

LC/MS method 2: MS (ESI): 294 [M+NH4]⁺, rt=2.62. ¹H-NMR (CDCl₃): δ (ppm) 13.16 (br s, 1H), 7.33 (s, 2H), 2.25 (s, 6H), 1.27 (s, 12H).

### (3) 3′-[1-(4-Chloro-3-methyl-phenyl)-ethylamino]-3,5-dimethyl-biphenyl-4-carboxylic acid, INT24

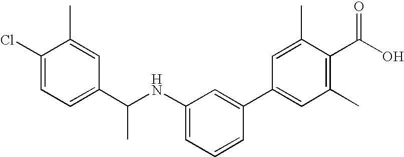

The intermediates INT22 (5.5 g, 16.94 mmol) and INT23 (6.08 g, 22.02 mmol) were dissolved in DME (170 mL) and sodium bicarbonate (9.96 g, 119 mmol) in water (100 mL) was added. Argon was bubbled through the solution for 10 minutes after which PdCl₂(PPh₃)₂ (0.595 g, 0.847 mmol) was added. The mixture was then refluxed under argon overnight. After cooling down, the DME was evaporated and the mixture diluted with water and acidified to pH=4-5 with 1M HCl. The mixture was then extracted three times with EtOAc and the combined organic layers were washed with brine, dried over sodium sulfate, filtered and concentrated. The residue was purified by chromatography on silica gel (cyclohexane/EtOAc) to give INT24 as a white solid.

LC/MS method 2: MS (ESI): 394 [M+H]⁺, rt=3.43. ¹H-NMR (DMSO-d₆) δ (ppm) 13.03 (br s, 1H), 7.37 (d, 1H), 7.30 (d, 1H), 7.21 (dd, 1H), 7.12 (s, 2H), 7.02 (t, 1H), 6.76-6.70 (m, 2H), 6.44 (dd, 3H), 6.24 (d, 1H), 4.52-4.45 (m, 1H), 2.29 (s, 9H), 1.40 (d, 3H).

### (4) ({3′-[1-(4-Chloro-3-methyl-phenyl)-ethylamino]-3,5-dimethyl-biphenyl-4-carbonyl}-amino)-acetic acid methyl ester, INT25

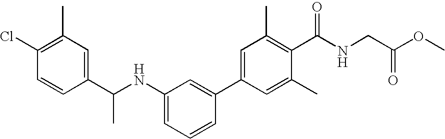

To a solution of the INT24 (100 mg, 0.254 mmol) and glycine methyl ester hydrochloride (47.8 mg, 0.381 mmol) in DMF (2.5 mL) was added DIPEA (98 mg, 0.762 mmol) followed by TBTU (98 mg, 0.305 mmol) and the resulting mixture was stirred at room temperature overnight. The DMF was evaporated under reduced pressure and the residue dissolved in EtOAc. The medium was washed with 5% aqueous sodium bicarbonate, brine/water (3 times) and brine, dried over sodium sulfate, filtered and concentrated. The residue was purified by chromatography on silica gel (cyclohexane/EtOAc) to give INT25 as a white solid.

LC/MS method 2: MS (ESI): 465 [M+H]⁺, rt=3.07. ¹H-NMR (DMSO-d₆): δ (ppm) 8.71 (t, 1H), 7.37 (d, 1H), 7.30 (d, 1H), 7.22 (dd, 1H), 7.09 (s, 2H), 6.71-6.60 (m, 1H), 6.45-6.42 (m, 2H), 6.24 (s, 1H), 4.51-4.44 (s, 1H), 3.98 (d, 2H), 3.66 (s, 3H), 2.29 (s, 3H), 2.27 (s, 6H), 1.40 (s, 3H).

### (5) ({3′-[1-(4-Chloro-3-methyl-phenyl)-ethylamino]-3,5-dimethyl-biphenyl-4-carbonyl}-amino)-acetic acid

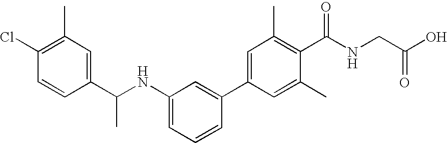

To a solution of INT25 (131 mg, 0.265 mmol) in THF/water (3 mL, 2:1) was added LiOH hydrate (22.2 mg, 0.529 mmol) and the resulting mixture was stirred at room temperature for 3 hours. The reaction mixture was diluted with EtOAc and water. 1M HCl (0.529 mL) was then added and the organic layer was separated, washed with brine, dried over sodium sulfate, filtered and concentrated. The residue was purified by chromatography on silica gel (cyclohexane/EtOAc) to give Example EX68 as a white solid.

LC/MS method 2: MS (ESI): 451 [M+H]⁺, rt=2.76. ¹H-NMR (DMSO-d₆): δ (ppm) 12.49 (br s, 1H), 8.58 (t, 1H), 7.37 (d, 1H), 7.30 (d, 1H), 7.22 (dd, 1H), 7.09 (s, 2H), 6.72-6.68 (m, 2H), 6.45-6.42 (m, 1H), 6.24 (s, 1H), 4.51-4.44 (s, 1H), 3.88 (d, 2H), 2.29 (s, 3H), 2.26 (s, 6H), 1.40 (s, 3H).

### Example EX69

### (S)-2-({3′-[1-(4-Chloro-3-methyl-phenyl)-ethylamino]-3,5-dimethyl-biphenyl-4-carbonyl}-amino)-3-methoxy-propionic acid

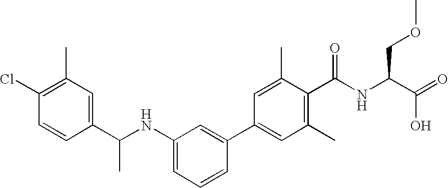

The title compound was prepared according to Scheme 2 following a procedure analogous to Example EX68 using (S)-2-amino-3-methoxy-propionic acid hydrochloride in step 3.

LC/MS Method 2: MS (ESI): 495 [M+H]⁺, rt=2.86 min.

### Example EX70

### (S)-6-Amino-2-({3′-[1-(4-chloro-3-methyl-phenyl)-ethylamino]-3,5-dimethyl-biphenyl-4-carbonyl}-amino)-hexanoic acid

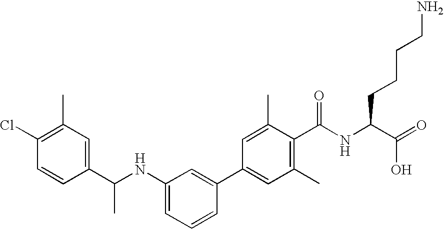

The title compound was prepared according to Scheme 2 following a procedure analogous to Example EX68 using N—BOC-lysine methyl ester hydrochloride in step 3 and with an additional HCl-induced BOC deprotection after step 4.

LC/MS Method 2: MS (ESI): 522 [M+H]⁺, rt=2.27 min.

### Example EX71

### 2-({3′-[(4-Chloro-3-methyl-phenyl)-ethylamino]-3,5-dimethyl-biphenyl-4-carbonyl}-amino)-2-methyl-propionic acid

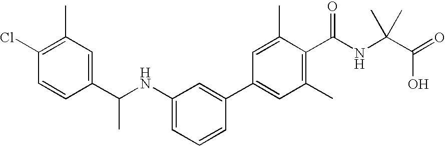

The title compound was prepared according to Scheme 2 following a procedure analogous to Example EX68 using methyl α-aminoisobutyrate hydrochloride in step 3.

LC/MS Method 2: MS (ESI): 479 [M+H]⁺, rt=2.94 min.

### Example EX72

### 1-({3′-[1-(4-Chloro-3-methyl-phenyl)-ethylamino]-3,5-dimethyl-biphenyl-4-carbonyl}-amino)-cyclobutanecarboxylic acid

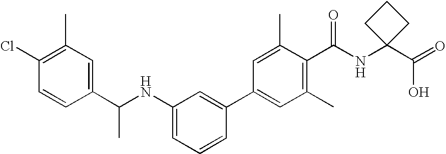

The title compound was prepared according to Scheme 2 following a procedure analogous to Example EX68 using ethyl 1-amino-1-cyclobutanecarboxylate hydrochloride in step 3.

LC/MS Method 2: MS (ESI): 491 [M+H]⁺, rt=2.96 min.

### Example EX73

### (S)-2-({3-[(R)-1-(4-Chloro-3-methyl-phenyl)-ethylamino]-3,5-dimethyl-biphenyl-4-carbonyl}-methyl-amino)-propionic acid

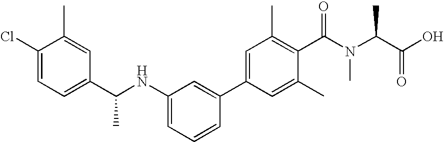

### (1) (3-Bromo-phenyl)-[(R)-1-(4-chloro-3-methyl-phenyl)-ethyl]-amine, INT26

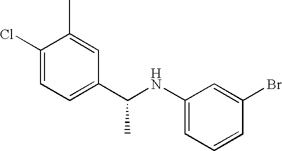

The title compound was obtained by preparative chiral separation of INT22 (Method B). Chiral HPLC method E: rt=7.4 min, n-heptane/EtOH/MeOH (60:30:10). Alternatively, the title compound can be obtained by the following procedure:

A mixture of INT4 (19.6 g, 116 mmol), 3-bromoaniline (20 g, 116 mmol), NaHCO₃ (48.8 g, 581 mmol) and 4 Å molecular sieves (100 g) in benzene was heated to reflux for 72 hours under an argon atmosphere. The mixture was filtered through celite and the celite pad was washed with dichloromethane. The filtrate was concentrated under reduced pressure. The remaining starting materials were removed by Kugelrohr distillation to leave the imine. This imine (11.45 g, 34.4 mmol) and (S)—N-(5-fluoro-2-hydroxybenzyl)-2-methylpropane-2-sulfinamide (1.69 g, 6.88 mmol, prepared according to: Pei, Dong; Wang, Zhouyu; Wei, Siyu; Zhang, Yu; Sun, Jian. Org. Lett. (2006), 8(25), 5913-5915) were dissolved in CH₂Cl₂ (400 mL) and the mixture was cooled to −20° C. Trichlorosilane (6.95 mL, 68.8 mmol) was added dropwise and the resulting mixture was stirred at −20° C. for 5 days. The mixture was quenched with saturated aqueous NaHCO₃ and extracted with EtOAc. The combined organic layers were washed with brine and dried over sodium sulfate, filtered and concentrated. The residue was purified by chromatography on silica gel (cyclohexane/EtOAc). The resulting product was further purified by formation of the HCl salt and washing it with Et₂O. The salt was then liberated to give INT26.

LC/MS method 1: (ESI): 326 [M+H]⁺, rt=1.71 min. ¹H-NMR (CDCl₃): δ (ppm) 7.27 (d, 1H), 7.19 (d, 1H), 7.09 (dd, 1H), 6.92 (t, 1H), 6.77-6.74 (t, 1H), 6.64 (s, 1H), 6.39-6.35 (m, 1H), 4.38 (q, 1H), 4.05 (s, 1H), 2.35 (s, 3H), 1.47 (d, 3H).

### (2) 3′-[(R)-1-(4-Chloro-3-methyl-phenyl)-ethylamino]-3,5-dimethyl-biphenyl-4-carboxylic acid hydrochloride, INT27

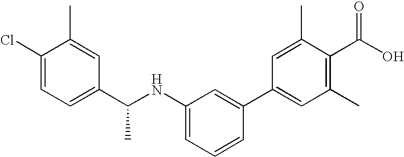

The intermediates INT26 (3.32 g, 10.23 mmol) and INT23 (3.67 g, 13.29 mmol) were dissolved in DME (100 mL) and sodium bicarbonate (6.01 g, 71.6 mmol) in water (70 mL) was added. Argon was bubbled through the solution for 10 minutes after which PdCl₂(PPh₃)₂ (0.359 g, 0.511 mmol) was added. The mixture was then refluxed under argon overnight. After cooling down, the DME was evaporated and the mixture diluted in water and acidified to pH=4-5 with 1M HCl. The mixture was then extracted three times with EtOAc and the combined organic layers were washed with brine, dried over sodium sulfate, filtered and concentrated. The residue was taken up in ether and 4M HCl in dioxane was added (10 mL) and the precipitate was collected by filtration and washed with ether to give INT27 which was used without further purification in the next step.

LC/MS method 1: MS (ESI): 394 [M+H]⁺, rt=1.53.

### (3) (S)-2-({3′-[(R)-1-(4-Chloro-3-methyl-phenyl)-ethylamino]-3,5-dimethyl-biphenyl-4-carbonyl}-methyl-amino)-propionic acid methyl ester, INT28

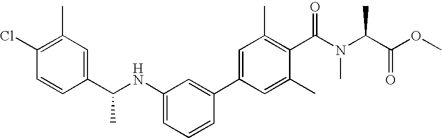

To a solution of the acid INT27 (500 mg, 1.16 mmol) and N-methylalanine methyl ester hydrochloride (357 mg, 2.32 mmol) in DMF (6 mL) was added DIPEA (1.01 mL, 5.81 mmol) followed by HATU (530 mg, 1.39 mmol) and the resulting mixture was stirred at room for 1 week. The DMF was evaporated under reduced pressure and the residue dissolved in EtOAc. The medium was washed with 5% aqueous sodium bicarbonate, brine/water (3 times) and brine, dried over sodium sulfate, filtered and concentrated. The residue was purified by chromatography on silica gel (cyclohexane/EtOAc) to give INT28 as a white solid.

LC/MS Method 2: Two rotamers were observed: MS (ESI): 493 [M+H]⁺, rt=3.17+3.23 min.

¹H-NMR (DMSO-d₆): major rotamer: δ (ppm) 7.39 (s, 1H), 7.32 (d, 1H), 7.24 (d, 1H), 7.15 (d, 2H), 7.04 (t, 1H), 6.76 (s, 1H), 6.74 (d, 1H), 6.46 (d, 1H), 6.26 (d, 1H), 4.98 (q, 1H), 4.53-4.48 (m, 1H), 3.68 (s, 3H), 2.69 (s, 3H), 2.29 (s, 3H), 2.20 (s, 3H), 2.18 (s, 3H), 1.43 (d, 3H), 1.41 (d, 3H).

### (4) (S)-2-({3′-[(R)-1-(4-Chloro-3-methyl-phenyl)-ethylamino]-3,5-dimethyl-biphenyl-4-carbonyl}-methyl-amino)-propionic acid

To a solution of INT28 (315 mg, 0.639 mmol) in THF/water was added LiOH hydrate (53.6 mg, 1.28 mmol) and the resulting mixture was stirred at room temperature for 3 hours. The reaction mixture was diluted with EtOAc and water. 1M HCl (1.28 mL) was then added and the organic layer was separated, washed with brine, dried over sodium sulfate, filtered and concentrated. The residue was purified by chromatography on silica gel (cyclohexane/EtOAc) to give Example EX73 as a white solid.

LC/MS Method 2: Two rotamers were observed: MS (ESI): 479 [M+H]⁺, rt=2.81+2.94 min.

¹H-NMR (DMSO-d₆): major rotamer: δ (ppm) 12.49 (br s, 1H), 7.39 (s, 1H), 7.33 (d, 1H), 7.24 (d, 1H), 7.14 (d, 2H), 7.04 (t, 1H), 6.76 (s, 1H), 6.74 (d, 1H), 6.46 (d, 1H), 6.26 (d, 1H), 5.03 (q, 1H), 4.54-4.48 (m, 1H), 2.67 (s, 3H), 2.29 (s, 3H), 2.20 (s, 3H), 2.17 (s, 3H), 1.41 (d, 3H), 1.40 (d, 3H).

### Example EX74

### (S)-2-({3′-[1-(4-Chloro-3-methyl-phenyl)-2,2,2-trifluoro-ethylamino]-3,5-dimethyl-biphenyl-4-carbonyl}-amino)-propionic acid

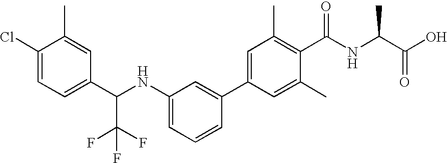

The title compound was prepared from INT30 (synthesis below) following the steps 3, 4 and 5 of the preparation of Example EX68 using alanine methyl ester hydrochloride in step 3.

LC/MS Method 2: MS (ESI): 517 [M+H]⁺, rt=2.89 min.

### (1) 1-(4-Chloro-3-methylphenyl)-2,2,2-trifluoroethanol, INT29

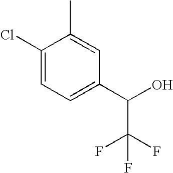

To a solution of 1-(4-chloro-3-methyl-phenyl)-2,2,2-trifluoro-ethanone (1 g, 4.49 mmol) in EtOH (10 mL) at 0° C. was added sodium borohydride (173 mg, 4.49 mmol) in portions and the resulting mixture was stirred at room temperature for 2 hours. The mixture was then cooled to 0° C. and 15 mL of 1N HCl were added. The EtOH was evaporated and the mixture was neutralized with saturated aqueous sodium bicarbonate and extracted with EtOAc. The organic layer was washed with brine, dried over sodium sulfate, filtered and concentrated to give INT29 as a white solid which was used without further purification.

MS (ESI): 223 [M−H]⁻. ¹H-NMR (CDCl₃): δ (ppm) 7.38 (d, 1H), 7.35 (s, 1H), 7.24 (d, 2H), 5.01-4.95 (m, 1H), 2.59 (d, 1H), 2.40 (s, 3H).

### (2) (3-Bromo-phenyl)-[1-(4-chloro-3-methyl-phenyl)-2,2,2-trifluoro-ethyl]-amine, INT30

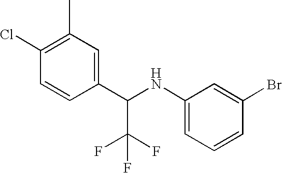

The alcohol INT29 (873 mg, 3.89 mmol) and 2,6-lutidine (500 mg, 4.67 mmol) were dissolved in DCM (3 mL) and cooled to 0° C. Trifluoromethanesulfonic anhydride (1.32 g, 4.67 mmol) was added dropwise and the mixture was stirred at room temperature for 3 hours. The resulting triflate and 3-bromoaniline (2.25 g, 12.84 mmol) were taken up in NMP (32.5 mL), and potassium carbonate (535 mg, 3.89 mmol) was added and the mixture was stirred at 65° C. overnight The mixture was then dissolved in EtOAc, washed with brine/water (1:1), 1M KHSO₄, brine, dried over sodium sulfate, filtered and concentrated. The residue was purified by chromatography on silica gel (cyclohexane/EtOAc) to give INT30 as a colorless oil.

LC/MS method 1: MS (ESI): 380 [M+H]⁺, rt=1.71 min. ¹H-NMR (CDCl₃): δ (ppm) 7.38 (d, 1H), 7.29 (s, 1H), 7.21 (d, 1H), 7.01 (t, 1H), 6.92-6.88 (m, 1H), 6.79 (t, 1H), 6.52 (dd, 1H), 4.85-4.77 (m, 1H), 4.35 (d, 1H), 2.39 (s, 3H).

### Example EX75

### (S)-2-({3′-[1-(4-Chloro-3-methyl-phenyl)-2,2-difluoro-ethylamino]-3,5-dimethyl-biphenyl-4-carbonyl}-amino)-propionic acid

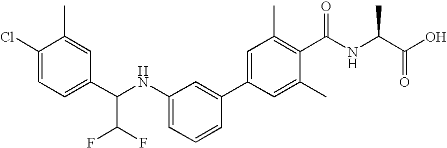

The title compound was prepared from INT31 (synthesis below) following the steps 3, 4 and 5 of the preparation of Example EX68 using alanine methyl ester hydrochloride in step 3.

LC/MS Method 2: MS (ESI): 501 [M+H]⁺, rt=2.68 min.

### [1-(4-Chloro-3-methyl-phenyl)-ethylidene]-(3-iodo-phenyl)-amine, INT31

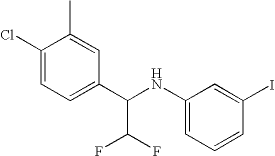

A mixture of 3-iodoaniline (10 g, 45.7 mmol), INT4 (7.70 g, 45.7 mmol), sodium bicarbonate (19.18 g, 228 mmol) and 4 Å molecular sieves (50 g) in benzene was heated to reflux for 4 days. The reaction mixture was filtered through Celite. The resulting Celite pad was thoroughly washed with DCM. The filtrate was concentrated in vacuo and the remaining starting materials were distilled off (130° C., 0.1 mbar) to leave the pure imine as a yellow oil. The imine (739 mg, 2 mmol) and sodium sulfate (200 mg, 1.41 mmol) were taken up in acetonitrile (20 mL). Selectfluor® (1.42 g, 4 mmol) was added and the mixture was stirred at 85° C. for 5 hours. After cooling to room temperature, MeOH (5 mL) was added followed by decaborane (244 mg, 2 mmol) and the mixture was stirred at room temperature overnight. The mixture was then concentrated, taken up in MeOH, filtered on Celite and concentrated. The residue was purified by chromatography on silica gel (cyclohexane/EtOAc) to give INT31 as a brown oil.

LC/MS method 1: MS (ESI): 408 [M+H]⁺, rt=1.67 min. ¹H-NMR (CDCl₃): δ (ppm) 7.35 (d, 1H), 7.25 (d, 1H), 7.15 (dd, 1H), 7.09-7.05 (m, 1H), 6.97 (t, 1H), 6.83 (t, 1H), 6.56 (dd, 1H), 5.94 (t, d, 1H), 4.64-4.55 (m, 1H), 4.37 (d, 1H), 2.38 (s, 3H).

Alternatively, agents of the invention may be prepared by a reaction sequence involving the coupling of a benzoic acid and an amino acid ester, Suzuki-type coupling with an appropriate boronic acid or ester, reductive amination with an appropriate ketone, and saponification of the ester followed by an optional deprotection step as shown in Scheme 3 below:

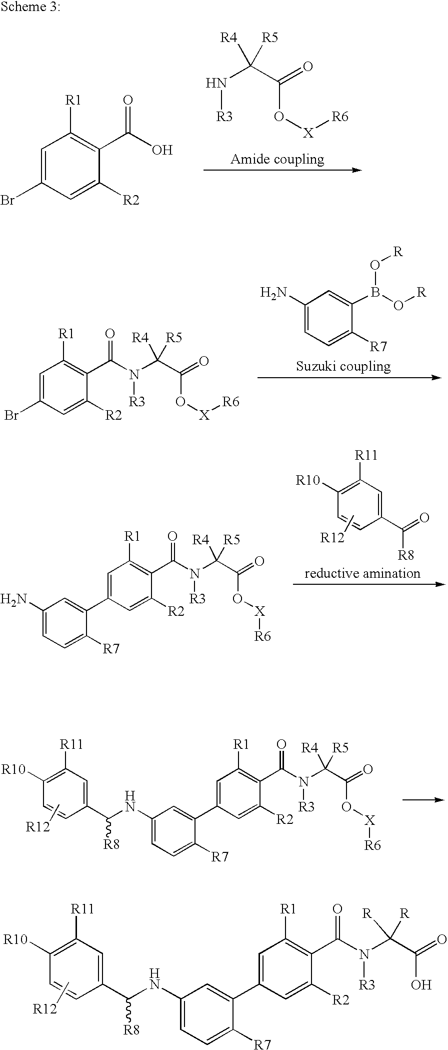

### Example EX76

### (S)-2-({3′-[1-(4-Chloro-3-methyl-phenyl)-ethylamino]-3,5-dimethyl-biphenyl-4-carbonyl}-methyl-amino)-propionic acid

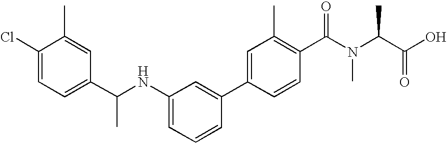

### (1) (S)-2-[(4-Bromo-2-methyl-benzoyl)-methyl-amino]propionic acid methyl ester, INT32

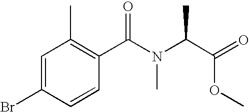

To a suspension of 4-bromo-2-methyl-benzoic acid (5.0 g, 23.25 mmol) in DCM (116 mL) a few drops of DMF and thionyl chloride (3.46 mL, 46.50 mmol) were added and the resulting mixture was refluxed for 1 hour. The mixture was then concentrated under reduced pressure and taken up in THF (116 mL). DIPEA (17.18 mL, 93.00 mmol) was then added, followed by N-methyl alanine methyl ester hydrochloride (4.29 g, 27.9 mmol) and the mixture was stirred at room temperature overnight. The mixture was diluted in EtOAc and washed with 1M HCl, saturated aqueous sodium bicarbonate, brine, dried over sodium sulfate, filtered and concentrated. The residue was purified by chromatography on silica gel (cyclohexane/EtOAc) to give INT32 as a white foam.

LC/MS method 2: MS (ESI): 316 [M+H]⁺, rt=2.56 min. ¹H-NMR (DMSO-d₆) major rotamer: δ (ppm) 7.53 (s, 1H), 7.44 (d, 1H), 7.10 (d, 1H), 4.94 (m, 2H), 3.67 (s, 3H), 2.68 (s, 3H), 2.21 (s, 3H), 1.42 (d, 3H).

### (2) (S)-2-[(3′-Amino-3-methyl-biphenyl-4-carbonyl)-methyl-amino]-propionic acid methyl ester, INT33

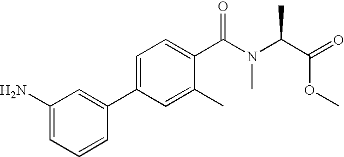

A flask was charged with INT32 (2 g, 6.37 mmol), (3-aminophenyl)-boronic acid (1.31 g, 9.55 mmol) and potassium phosphate (4.05 g, 19.1 mmol) and was flushed with Argon. Degassed THF (6 mL) was added followed by water (64 μL). A separate flask was charged with Pd(OAc)₂ (223 mg, 0.32 mmol) and 2-dicyclohexylphosphino-2′,6′-dimethoxybiphenyl (131 mg, 0.32 mmol) and flushed with Argon and degassed THF (3 mL) was added. The resulting suspension was stirred for 5 minutes and added to the previously prepared mixture and the resulting suspension was stirred at room temperature overnight. Ether (150 mL) was added and the mixture was filtered through Celite and concentrated. The residue was purified by chromatography on silica gel (cyclohexane/EtOAc) to give INT33 as a pale yellow solid.

LC/MS method 1: MS (ESI): 327 [M+H]⁺, rt=0.75 min. ¹H-NMR (CDCl₃) major rotamer: δ (ppm) 7.43-7.36 (m, 2H), 7.22 (t, 2H), 6.99-6.94 (m, 1H), 6.90-6.86 (m, 1H), 6.69 (dd, 1H), 5.44 (q, 1H), 3.78 (s, 3H), 2.81 (s, 3H), 2.38 (s, 3H), 1.53 (d, 3H).

### (3) (S)-2-({3′-[1-(4-Chloro-3-methyl-phenyl)-ethylamino]-3-methyl-biphenyl-4-carbonyl}-methyl-amino)-propionic acid methyl ester, INT34

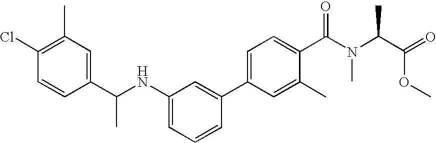

The title compound INT34 was prepared by reductive amination of INT33 with INT4 using a similar procedure as described for INT3.

LC/MS method 1: MS (ESI): 479 [M+H]⁺, rt=1.53 min. ¹H-NMR (CDCl₃) major rotamer: δ (ppm) 7.35-7.25 (m, 4H), 7.22-7.11 (m, 3H), 6.88-6.83 (m, 1H), 6.88-6.83 (m, 1H), 6.72-6.67 (m, 1H), 6.50-6.45 (m, 1H), 5.47 (q, 1H), 4.48 (q, 1H), 3.78 (s, 3H), 2.80 (s, 3H), 2.36 (s, 3H), 2.35 (s, 3H), 1.53 (d, 3H), 1.52 (d, 3H).

### (4) (S)-2-({3′-[1-(4-Chloro-3-methyl-phenyl)-ethylamino]-3-methyl-biphenyl-4-carbonyl}-methyl-amino)-propionic acid

The title compound was prepared from INT34 by saponification following an analogous procedure as described in step 4 of Example EX1.

LC/MS method 2: MS (ESI): 465 [M+H]⁺, rt=3.26 min. ¹H-NMR (DMSO-d₆) major rotamer: δ (ppm) 7.37 (s, 1H), 7.35-7.25 (m, 3H), 7.224 (d, 1H), 7.14 (d, 1H), 7.04 (t, 1H), 6.77 (s, 1H), 6.74 (d, 1H), 6.45 (d, 1H), 6.29-6.22 (m, 1H), 4.48 (q, 1H), 4.57-4.47 (m, 1H), 2.70 (s, 3H), 2.28 (s, 3H), 2.25 (s, 3H), 1.40 (d, 6H).

### Example EX77

### (S)-2-({3′-[1-(4-Chloro-phenyl)-ethylamino]-3-methyl-biphenyl-4-carbonyl}-methyl-amino)-propionic acid

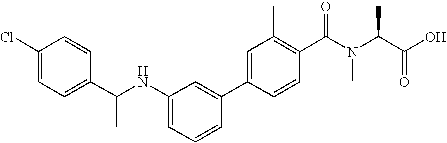

The title compound was prepared according to Scheme 3 following a procedure analogous to Example EX76 using 1-(4-chloro-phenyl)-ethanone in step 3.

LC/MS Method 2: MS (ESI): 451 [M+H]⁺, rt=3.08 min.

### Example EX78

### (S)-2-({3′-[1-(3,4-Dichloro-phenyl)-ethylamino]-3-methyl-biphenyl-4-carbonyl}-methyl-amino)-propionic acid

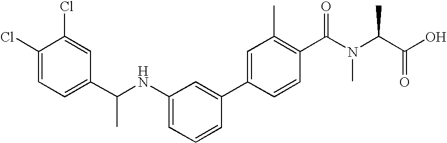

The title compound was prepared according to Scheme 3 following a procedure analogous to Example EX76 using 1-(3,4-dichloro-phenyl)-ethanone in step 3.

LC/MS Method 2: MS (ESI): 485 [M+H]⁺, rt=3.26 min.

### Example EX79

### 3-({3′-[1-(4-Chloro-3-methyl-phenyl)-ethylamino]-3,5-dimethyl-biphenyl-4-carbonyl}-amino)-azetidine-3-carboxylic acid

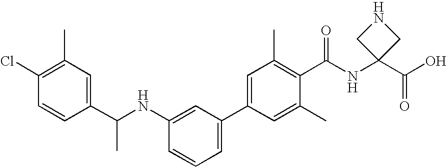

The title compound was prepared according to Scheme 3 following a procedure analogous to Example EX76 using 3-amino-azetidine-1,3-dicarboxylic acid 1-tert-butyl ester 3-ethyl ester in step 1. An additional TFA-induced BOC cleavage after step 4 gave EX79.

LC/MS Method 2: MS (ESI): 492 [M+H]⁺, rt=2.29 min.

### Example EX80

### (S)-2-[(3′-{[1-(4-Chloro-3-methyl-phenyl)-ethyl]-methyl-amino}-3,5-dimethyl-biphenyl-4-carbonyl)-amino]-propionic acid

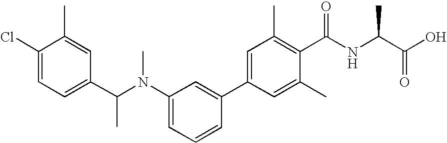

### (1) (S)-2-[(3′-{[1-(4-Chloro-3-methyl-phenyl)-ethyl]-methyl-amino}-3,5-dimethyl-biphenyl-4-carbonyl)-amino]-propionic acid methyl ester, INT35

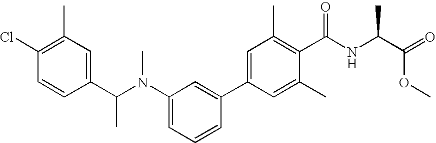

To a solution of INT2 (100 mg, 0.306 mmol) and INT4 (57 mg, 0.337 mmol) in MeOH (3 mL) was added decaborane (19.7 mg, 0.153 mmol) and the resulting mixture was stirred at room temperature overnight. Then, formaldehyde (30% in water, 91 μL) was added and the resulting mixture was further stirred at room temperature for 5 hours. The mixture was then concentrated under reduced pressure and the residue was purified by chromatography on silica gel (cyclohexane/EtOAc) to give INT35 as a white solid.

LC/MS method 2: MS (ESI): 493 [M+H]⁺, rt=3.89 min. ¹H-NMR (DMSO-d₆): δ (ppm) 8.72 (d, 1H), 7.34 (d, 1H), 7.29 (d, 1H), 7.24 (s, 2H), 7.20 (d, 1H), 7.12 (dd, 1H), 6.94 (s, 1H), 6.86 (d, 1H), 6.80 (dd, 1H), 5.18 (q, 1H), 4.48-4.41 (m, 1H), 3.66 (s, 3H), 2.72 (s, 3H), 2.31 (s, 2.28 (s, 6H), 1.49 (d, 3H), 1.34 (d, 3H).

### (2) (S)-2-[(3′-{[1-(4-Chloro-3-methyl-phenyl)-ethyl]-methyl-amino}-3,5-dimethyl-biphenyl-4-carbonyl)-amino]-propionic acid

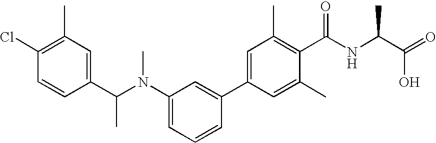

The title compound was prepared from INT35 by saponification following an analogous procedure as described in step 4 of Example EX1.

LC/MS method 2: MS (ESI): 479 [M+H]⁺, rt=3.53 min. ¹H-NMR (DMSO-d₆): δ (ppm) 12.45 9s, 1H), 8.55 (d, 1H), 7.34 (d, 1H), 7.29 (d, 1H), 7.22 (s, 2H), 7.21 (d, 1H), 7.12 (d, 1H), 6.93 (s, 1H), 6.86 (d, 1H), 6.80 (d, 1H), 5.17 (q, 1H), 4.40-4.35 (m, 1H), 2.72 (s, 3H), 2.31 (s, 3H), 2.27 (s, 6H), 1.49 (d, 3H), 1.32 (d, 3H).

### Example EX81

### 1-({3′-[(R)-1-(4-Chloro-3-methyl-phenyl)-ethylamino]-3,5-dimethyl-biphenyl-4-carbonyl}-methyl-amino)-cyclopropanecarboxylic acid

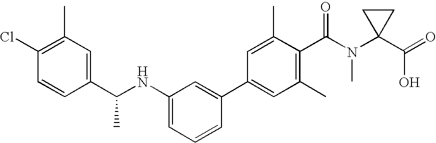

### (1) 1-(4-Bromo-2,6-dimethyl-benzoylamino)-cyclopropanecarboxylic acid ethyl ester, INT36

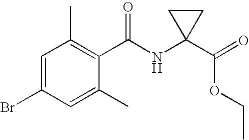

To a suspension of 4-bromo-2,6-dimethyl-benzoic acid (864 mg, 3.77 mmol) in DCM (19 mL) a few drops of DMF and thionyl chloride (0.561 mL, 7.44 mmol) were added and the resulting mixture was refluxed for 1 hour. The mixture was then concentrated under reduced pressure and taken up in THF (19 mL). DIPEA (3.49 mL, 18.87 mmol) was then added, followed by ethyl 1-amino-1-cyclopropanecarboxylate hydrochloride (1.25 g, 7.44 mmol) and the mixture was stirred at room temperature overnight. The mixture was diluted in EtOAc, washed with 1M HCl, saturated aqueous sodium bicarbonate and brine, dried over sodium sulfate, filtered and concentrated. The residue was purified by chromatography on silica gel (cyclohexane/EtOAc) to give INT36 as a white foam.

LC/MS method 2: MS (ESI): 340 [M+H]⁺, rt=2.19 min. ¹H-NMR (DMSO-d₆): δ (ppm) 8.94 (s, 1H), 7.27 (s, 211, 4.08 (q, 2H), 2.22 (s, 6H), 1.42 (q, 2H), 1.18 (t, 3H), 1.09 (q,

### (2) 1-[(4-Bromo-2,6-dimethyl-benzoyl)-methyl-amino]-cyclopropanecarboxylic acid ethyl ester, INT37

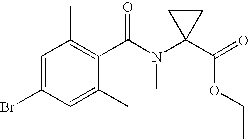

To a suspension of sodium hydride (60° AD in mineral oil, 0.194 g, 4.85 mmol) in DMF (5 mL) at 0° C. was added dropwise a solution of INT36 (1.5 g, 4.41 mmol) in DMF (17 mL) and the resulting mixture was stirred at this temperature for 1 hour. Then, methyl iodide (0.751 g, 5.29 mmol) was added and the mixture was stirred at room temperature for 18 hours. The mixture was then poured on ice and extracted with EtOAc. The organic layer was washed with brine, dried over sodium sulfate, filtered and concentrated. The residue was purified by chromatography on silica gel (cyclohexane/EtOAc) to give INT37 as a white foam.

LC/MS method 2: MS (ESI): 354 [M+H]⁺, rt=2.63 min. ¹H-NMR (DMSO-d₆): δ (ppm) 7.31 (s, 2H), 4.01 (q, 2H), 2.71 (s, 3H), 2.15 (s, 6H), 1.50 (m, 2H), 1.33 (m, 2H), 1.18 (t,

### (3) 1-{[2,6-Dimethyl-4-(4,4,5,5-tetramethyl-[1,3,2]dioxaborolan-2-yl)-benzoyl]-methyl-amino}-cyclopropanecarboxylic acid ethyl ester, INT38

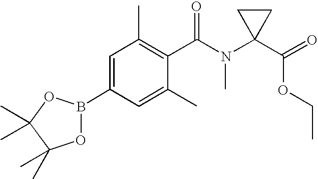

Argon was bubbled for 15 min through a mixture of bis(pinacoloto)diboron (326 mg, 1.28 mmol), INT37 (379 mg, 1.07 mmol) and potassium acetate (637 mg, 6.42 mmol) in dioxane (12 mL). Then PdCl₂(dppf) (43.7 mg, 0.054 mmol) was added and the resulting mixture was stirred at 80° C. overnight. After cooling down, the mixture was concentrated and partitioned between ether and 5% aqueous sodium bicarbonate. The organic layer was further extracted with 5% aqueous sodium bicarbonate and the combined aqueous layers were cooled to 0° C. and acidified to pH=2 with 2M HCl. The mixture was then extracted three times with EtOAc and the combined organic layers were washed with brine, dried over sodium sulfate, filtered and concentrated. The residue was purified by chromatography on silica gel (cyclohexane/EtOAc) to give INT38 as a beige solid.

MS (ESI): 402 [M+H]⁺. ¹H-NMR (DMSO-d₆): δ (ppm) 7.36 (s, 2H), 4.13 (q, 2H), 2.70 (s, 3H), 2.17 (s, 6H), 1.52 (m, 2H), 1.33 (m, 2H), 1.29 (s, 12H), 1.18 (t, 3H).

### (4) 1-({3′-[(R)-1-(4-Chloro-3-methyl-phenyl)-ethylamino]-3,5-dimethyl-biphenyl-4-carbonyl}-methyl-amino)-cyclopropanecarboxylic acid ethyl ester, INT39

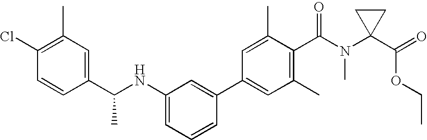

The intermediates INT38 (716 mg, 1.85 mmol) and INT26 (500 mg, 1.54 mmol) were dissolved in DME (90 mL) and sodium bicarbonate (906 mg, 10.8 mmol) in water (9 mL) was added. Argon was bubbled through the solution for 10 minutes after which PdCl₂(PPh₃)₂ (54.1 mg, 0.077 mmol) was added. The mixture was then refluxed under argon overnight. After cooling down, the DME was evaporated and the mixture diluted with water and extracted three times with EtOAc. The combined organic layers were washed with brine, dried over sodium sulfate, filtered and concentrated. The residue was purified by chromatography on silica gel (cyclohexane/EtOAc) to give INT39 as a yellow foam.

LC/MS method 2: MS (ESI): 519 [M+H]⁺, rt=3.63 min. ¹H-NMR (DMSO-d₆): δ (ppm) 7.39 (s, 1H), 7.32 (d, 1H), 7.23 (dd, 1H), 7.14 (s, 1H), 7.13 (s, 1H), 7.04 (t, 1H), 6.75 (s, 1H), 6.73 (d, 1H), 6.46 (d, 1H), 6.26 (d, 1H), 4.53-4.48 (m, 1H), 4.13 (q, 2H), 2.74 (s, 3H), 2.29 (s, 3H), 2.19 (s, 6H), 1.52 (m, 2H), 1.40 (d, 3H), 1.33 (m, 2H), 1.21 (t, 3H).

### (5) 1-({3′-[(R)-1-(4-Chloro-3-methyl-phenyl)-ethylamino]-3,5-dimethyl-biphenyl-4-carbonyl}-methyl-amino)-cyclopropanecarboxylic acid

To a solution of INT39 (62.7 mg, 0.121 mmol) in THF/water (3 mL) was added LiOH hydrate (61.0 mg, 1.45 mmol) and the resulting mixture was refluxed for 3 hours. The reaction mixture was cooled down and diluted with EtOAc and water. 1M HC (1.45 mL) was then added and the organic layer was separated, washed with brine, dried over sodium sulfate, filtered and concentrated. The residue was purified by chromatography on silica gel (cyclohexane/EtOAc) to give Example EX81 as a white solid.

LC/MS method 2: MS (ESI): [M+H]⁺=491, rt=3.31 min. ¹H-NMR (DMSO-d₆): δ (ppm) 12.65 (brs, 1H), 7.39 (s, 1H), 7.32 (d, 1H), 7.23 (dd, 1H), 7.13 (s, 1H), 7.12 (s, 1H), 7.04 (t, 1H), 6.75 (s, 1H), 6.73 (d, 1H), 6.45 (d, 1H), 6.25 (d, 1H), 4.54-4.47 (m, 1H), 2.73 (s, 3H), 2.29 (s, 3H), 2.18 (s, 6H), 1.41 (m, 2H), 1.39 (d, 3H), 1.27 (m, 2H).

Alternatively, agents of the invention may be prepared by a reaction sequence involving a Buchwald-type amination of an appropriately substituted aryl halide with an appropriate benzylic amine, Suzuki-type coupling with an appropriate boronic acid or ester, amide coupling of an amino acid ester, and saponification of the ester followed by an optional deprotection step, as shown in Scheme 4 below:

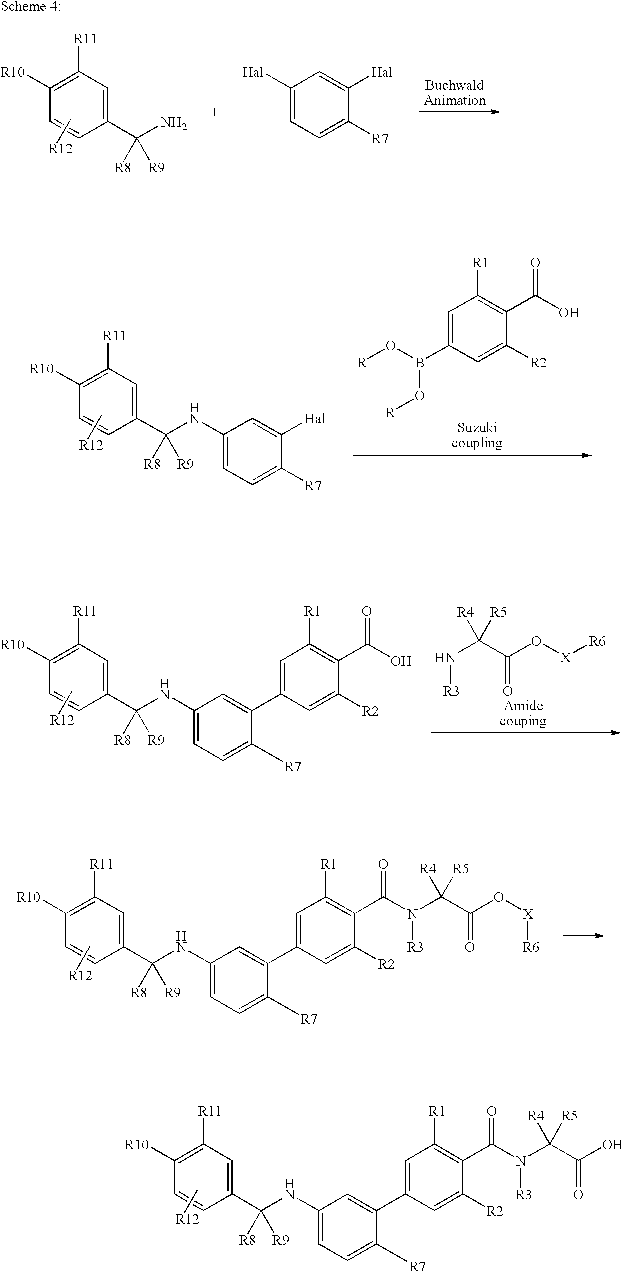

### Example EX82

### (S)-2-({3′-[1-(4-Chloro-3-methyl-phenyl)-1-methyl-ethylamino]-3,5-dimethyl-biphenyl-4-carbonyl}-amino)-propionic acid

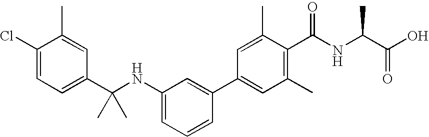

### (1) 2-(4-Chloro-3-methyl-phenyl)-propan-2-ol, INT40

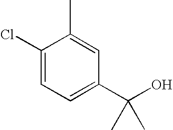

Methyl 4-chloro-3-methylbenzoate (12 g, 65 mmol) was dissolved in 250 mL of THF and cooled in an ice-bath. A solution of MeMgBr (3M in ether, 87 mL, 260 mmol) was added slowly, then the ice-bath was removed and the mixture was stirred at room temperature for 2 hours. To complete the reaction, the ether was distilled off and the mixture was heated under reflux for 14 hours. The reaction was then cooled and quenched with methanol and water, acidified with 2M HCl and extracted twice with ether. The organic layers were combined, dried over sodium sulfate, filtered and evaporated. The crude was purified by chromatography on silica gel using cyclohexane and ethyl acetate (from 5% to 50%).

¹H-NMR (CDCl₃): b (ppm) 7.34 (d, 1H), 7.28 (d, 1H), 7.21 (dd, 1H), 2.37 (s, 3H), 1.54 (s, 6H).

### (2) 4-(1-Azido-1-methyl-ethyl)-1-chloro-2-methyl-benzene, INT41

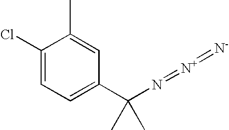

A flask was charged with sodium azide (4.22 g, 65 mmol) and chloroform (250 mL). The mixture was cooled in an ice-salt bath to −5° C. TFA (12.52 mL, 162 mmol) was added over 3 minutes, followed by a solution of the alcohol INT40 (6 g, 32.5 mmol) in 10 mL of chloroform while the temperature was kept below 0° C. The formed slurry was stirred for one hour, then allowed to warm up to room temperature over night. After that time, the thick slurry had turned into a cloudy solution. The mixture was treated with 25 mL of concentrated ammonia and 50 mL of water. The chloroform layer was separated and the aqueous layer was extracted once more with chloroform. The combined organic extracts were washed with water and brine, dried over sodium sulfate, filtered and evaporated. The crude was purified by chromatography on silica gel using cyclohexane and DCM (from 1% to 10%).

UPLC: rt=2.34 min. ¹H-NMR (CDCl₃): δ (ppm) 7.31 (d, 1H), 7.29 (s, 1H), 7.19 (dd, 1H), 2.39 (s, 3H), 1.60 (d, 6H).

### (3) 1-(4-Chloro-3-methyl-phenyl)-1-methyl-ethylamine, INT42

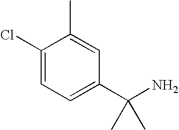

A solution of azide INT41 (4.58 g, 21.84 mmol) in 100 mL of methanol was hydrogenated at room temperature under atmospheric pressure for 6 hours in the presence of platinum dioxide hydrate (268 mg, 1.1 mmol). The reaction was monitored by UPLC. At the end of the reaction, the initially brown and finely dispersed catalyst was turning black and was clotting. The mixture was filtered through Celite and evaporated. The crude was used without further purification.

UPLC: rt=0.75 min. ¹H-NMR (CDCl₃): δ (ppm) 7.37 (d, 1H), 7.25 (m, 2H), 2.37 (s, 3H), 1.93 (br s, 2H), 1.47 (s, 6H).

### (4) [1-(4-Chloro-3-methyl-phenyl)-1-methyl-ethyl]-(3-iodo-phenyl)-amine, INT43

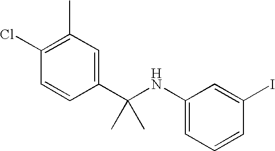

A suspension of amine INT42 (2.8 g, 15.24 mmol) and potassium tert-butoxide (5.99 g, 53.4 mmol) in DME (25 mL) was purged with argon for 5 minutes. Then, PEPPSI-IPr® (207 mg, 0.305 mmol) and 1,3-diiodobenzene (5.03 g, 15.24 mmol) were added and the flask was closed. The mixture was stirred at room temperature over the weekend. The crude reaction mixture was treated with 50 mL of ether and filtered. The residue was washed with more ether (30 mL) and the combined filtrates were evaporated. The crude was purified by chromatography on silica gel using cyclohexane and DCM (from 1% to 5%).

UPLC: rt=2.87 min. ¹H-NMR (DMSO-d₆): δ (ppm) 7.15-7.35 (m, 3H), 6.93 (m, 1H), 6.78 (m, 1H), 6.67 (t, 1H), 6.16 (dd, 1H), 4.02 (br s, 1H), 2.36 (s, 3H), 1.59 (s, 6H).

### (5) 3′-[1-(4-Chloro-3-methyl-phenyl)-1-methyl-ethylamino]-3,5-dimethyl-biphenyl-4-carboxylic acid, INT44

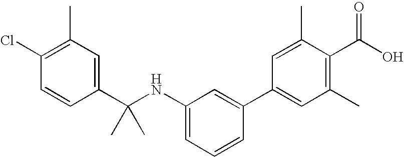

A suspension of intermediate INT43 (1 g, 2.59 mmol) and sodium bicarbonate (1.2 M solution, 15.1 mL, 18.15 mmol) in 40 mL of DME was purged with argon for 5 minutes. The flask was placed in a heat bath at 100° C., then Pd(PPh₃)₄ (30 mg, 0.026 mmol) was added, followed by a solution of intermediate INT23 (931 mg, 3.37 mmol) from step 2 of Example EX68 in 5 mL of DME over a period of about 15 minutes. After 2 hours, another portion (20 mg) of catalyst was added together with 3 mL of sodium bicarbonate solution and stirring was continued for 2 hours. The mixture was then allowed to cool to room temperature and was placed in a separatory funnel. The dark brown bottom layer was removed and the DME layer was evaporated. Water was added and the pH was adjusted to 4-5. It was then extracted with ethyl acetate, dried over sodium sulfate, filtered and evaporated. The crude was dissolved in DCM and treated with an excess of a 2M HCl solution in ether. Upon evaporation a precipitate was formed which was filtered-off and dried. This crude hydrochloride salt of the title compound was used without further purification.

LC/MS Method 1: MS (ESI): 408 [M+H]⁺, rt=1.45 min.

UPLC: rt=2.34 min.

### (6) (S)-2-({3′-[1-(4-Chloro-3-methyl-phenyl)-1-methyl-ethylamino]-3,5-dimethyl-biphenyl-4-carbonyl}-amino)-propionic acid methyl ester, INT45

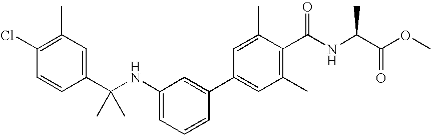

A solution of intermediate INT44 (376 mg, 0.783 mmol), alanine methyl ester hydrochloride (164 mg, 1.175 mmol), TBTU (302 mg, 0.94 mmol) and DIEA (0.479 mL, 2.74 mmol) in 5 mL of DMF was stirred at room temperature overnight. The DMF was evaporated and water was added. The pH was adjusted to about 4 and the aqueous layer was extracted twice with ethyl acetate, dried over sodium sulfate, filtered and evaporated. The crude was purified by chromatography on silica gel using cyclohexane and ethyl acetate (from 5% to 25%).

LC/MS Method 1: MS (ESI): 493.5 [M+H]⁺, rt=1.48 min. ¹H-NMR (CDCl₃): δ (ppm) 7.39 (s, 1H), 7.3 (s, 2H), 7.06 (t, 1H), 6.97 (s, 2H), 6.81 (d, 1H), 6.46 (m, 1H), 6.31 (dd, 1H), 6.23 (d, 1H), 4.85 (m, 1H), 4.11 (br s, 1H), 3.79 (s, 3H), 2.38 (s, 3H), 2.33 (s, 6H), 1.64 (s, 6H), 1.51 (d, 3H).

### (7) (S)-2-({3′-[1-(4-Chloro-3-methyl-phenyl)-1-methyl-ethylamino]-3,5-dimethyl-biphenyl-4-carbonyl}-amino)-propionic acid

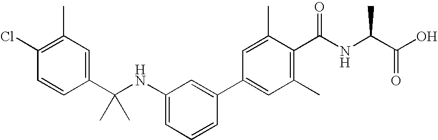

A solution of ester INT45 (265 mg, 0.537 mmol) in 20 mL of THF was treated with a 1M LiOH solution (2.15 mL, 2.15 mmol) and stirred vigorously overnight. Then, 2M HCl (1.1 mL) was added and most of the THF was evaporated. The residue was diluted with water, the pH was adjusted to 4 with a few drops of 0.2M HCl and it was extracted twice with ethyl acetate. The organic extracts were dried over sodium sulfate, filtered and evaporated to give the title compound Example EX82 as a white foam.

LC/MS Method 2: MS (ESI): 479 [M+H]⁺, rt=2.91 min. ¹H-NMR (DMSO-d₆): δ (ppm) 12.49 (br s, 1H), 8.59 (d, 1H), 7.46 (s, 1H), 7.37 (d, 1H), 7.3 (d, 1H), 6.98 (t, 1H), 6.90 (s, 2H), 6.68 (d, 1H), 6.44 (s, 1H), 6.33 (d, 1H), 6.06 (s, 1H), 4.37 (m, 1H), 2.33 (s, 3H), 2.22 (s, 6H), 1.56 (s, 6H), 1.32 (d, 3H).

### Example EX83

### (S)-2-({3′-[1-(4-Chloro-3-methyl-phenyl)-1-methyl-ethylamino]-3,5-dimethyl-biphenyl-4-carbonyl}-methyl-amino)-propionic acid

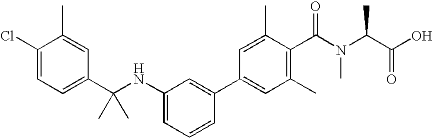

The title compound was prepared following a procedure analogous to Example EX82 using intermediate INT44 and N-methyl alanine methyl ester hydrochloride in step 6.

LC/MS Method 2: MS (ESI): 493 [M+H]⁺, rt=3.14 min.

### Example EX84

### 1-({3′-[1-(4-Chloro-3-methyl-phenyl)-1-methyl-ethylamino]-3,5-dimethyl-biphenyl-4-carbonyl}-amino)-cyclopropanecarboxylic acid

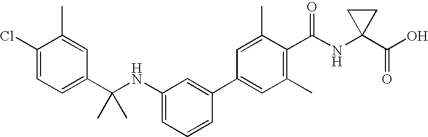

The title compound was prepared following a procedure analogous to Example EX82 using intermediate INT44 and 1-amino-cyclopropanecarboxylic acid ethyl ester in step 6.

LC/MS Method 2: MS (ESI): 491 [M+H]⁺, rt=3.02 min.

### Example EX85

### 1-({3′-[1-(4-Chloro-3-methyl-phenyl)-1-methyl-ethylamino]-3,5-dimethyl-biphenyl-4-carbonyl}-methyl-amino)-cyclopropanecarboxylic acid

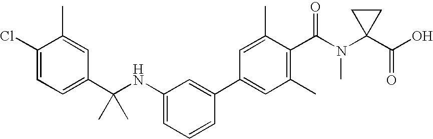

The title compound was prepared following a procedure analogous to Example EX81 using intermediate INT38 and intermediate INT43 (from step 4 of Example EX82) in step 4.

LC/MS Method 2: MS (ESI): 505 [M+H]⁺, rt=3.20 min.

### Example EX86

### (S)-2-({5′-[1-(4-Chloro-3-methyl-phenyl)-propylamino]-2′-fluoro-3-methyl-biphenyl-4-carbonyl}-methyl-amino)-propionic acid

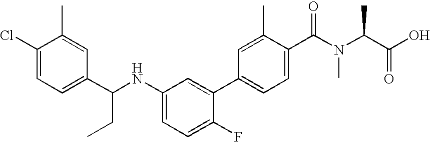

The title compound was prepared according to Scheme 1 following a procedure analogous to Example EX1 using 4-bromo-2-methyl-benzoic acid and (2-fluoro-5-amino-phenyl)-boronic acid in step 1, N-methyl alanine methyl ester hydrochloride in step 2 and INT8 in step 3.

LC/MS Method 2: MS (ESI): 497 [M+H]⁺, rt=3.05 min.

### Example EX87

### (S)-2-({3-Chloro-5′-[1-(4-chloro-3-methyl-phenyl)-ethylamino]-2′-fluoro-biphenyl-4-carbonyl}-methyl-amino)-propionic acid

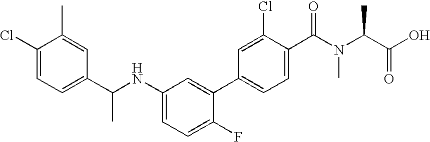

The title compound was prepared according to Scheme 1 following a procedure analogous to Example EX1 using 4-bromo-2-chloro-benzoic acid and (2-fluoro-5-amino-phenyl)-boronic acid in step 1, N-methyl alanine methyl ester hydrochloride in step 2 and INT4 in step 3.

LC/MS Method 2: MS (ESI): 503 [M+H]⁺, rt=2.97 min.

### Example EX88

### (S)-2-({3′-[1-(4-chloro-3-methyl-phenyl)-propylamino]-2′-fluoro-biphenyl-4-carbonyl}-methyl-amino)-propionic acid

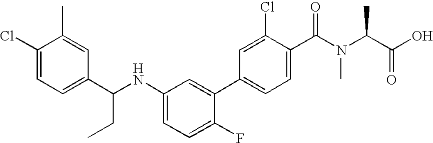

The title compound was prepared according to Scheme 1 following a procedure analogous to Example EX1 using 4-bromo-2-chloro-benzoic acid and (2-fluoro-5-amino-phenyl)-boronic acid in step 1, N-methyl alanine methyl ester hydrochloride in step 2 and INT8 in step 3.

LC/MS Method 2: MS (ESI): 517 [M+H]⁺, rt=3.12 min.

### Example EX89

### (S)-2-({5′-[1-(4-Chloro-3-methyl-phenyl)-propylamino]-2′-fluoro-3-trifluoromethyl-biphenyl-4-carbonyl}-methyl-amino)-propionic acid

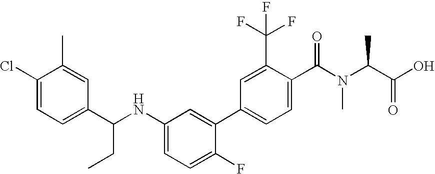

The title compound was prepared according to Scheme 1 following a procedure analogous to Example EX62 using (2-fluoro-5-amino-phenyl)-boronic acid in step 1, N-methyl alanine methyl ester hydrochloride in step 3 and INT8 in step 4.

UPLC: rt=2.39 min. MS (ESI): 551 [M+H]⁺.

### Example EX90

### (S)-2-({3′-[1-(4-Chloro-3-methyl-phenyl)-2-methyl-propylamino]-3,5-dimethyl-biphenyl-4-carbonyl}-amino)-propionic acid

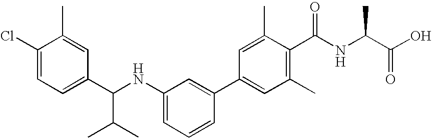

The title compound was prepared according to Scheme 1 following a procedure analogous to Example EX1 using 1-(4-chloro-3-methyl-phenyl)-2-methyl-propan-1-one in step 3.

LC/MS Method 2: MS (ESI): 493 [M+H]⁺, rt=3.14 min.

### Example EX91

### (S)-2-({3′-[1-(4-Chloro-3-methyl-phenyl)-2,2,2-trifluoro-ethylamino]-3-trifluoromethyl-biphenyl-4-carbonyl}-methyl-amino)-propionic acid

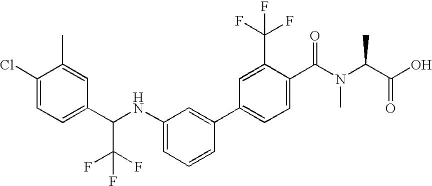

The title compound was prepared following a procedure analogous to steps 3, 4 and 5 of Example EX68 using intermediate INT30 and 4-(4,4,5,5-tetramethyl-[1,3,2]dioxaborolan-2-yl)-2-trifluoromethyl-benzoic acid methyl ester in step 3 (followed by LiOH-induced ester hydrolysis), and N-methyl alanine methyl ester hydrochloride in step 4.

LC/MS Method 2: MS (ESI): 573 [M+H]⁺, rt=3.06 min.

### Example EX92

### (S)-2-({3′-[1-(4-Chloro-3-methyl-phenyl)-2,2,2-trifluoro-ethylamino]-3-trifluoromethyl-biphenyl-4-carbonyl}-amino)-propionic acid

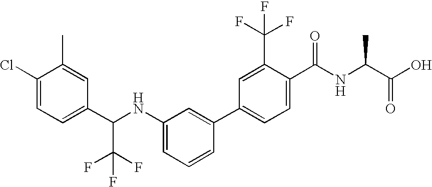

The title compound was prepared following a procedure analogous to steps 3, 4 and 5 of Example EX68 using intermediate INT30 and 4-(4,4,5,5-tetramethyl-[1,3,2]dioxaborolan-2-yl)-2-trifluoromethyl-benzoic acid methyl ester in step 3 (followed by LiOH-induced ester hydrolysis), and alanine methyl ester hydrochloride in step 4.

LC/MS Method 2: MS (ESI): 559 [M+H]⁺, rt=2.88 min.

### Example EX93

### 1-({3′-[1-(4-Chloro-3-methyl-phenyl)-2,2,2-trifluoro-ethylamino]-3-trifluoromethyl-biphenyl-4-carbonyl}-methyl-amino)-cyclopropanecarboxylic acid

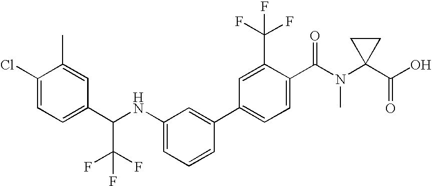

The title compound was prepared following a procedure analogous to steps 3, 4 and 5 of Example EX68 using intermediate INT30 and 4-(4,4,5,5-tetramethyl-[1,3,2]dioxaborolan-2-yl)-2-trifluoromethyl-benzoic acid methyl ester in step 3 (followed by LiOH-induced ester hydrolysis), and 1-methylamino-cyclopropanecarboxylic acid ethyl ester hydrochloride in step 4.

LC/MS Method 2: MS (ESI): 571 [M+H]⁺, rt=2.91 min.

### Example EX94

### (S)-2-({3′-[1-(4-chloro-3-methyl-phenyl)-2,2,2-trifluoro-ethylamino]-biphenyl-4-carbonyl}-amino)-propionic acid

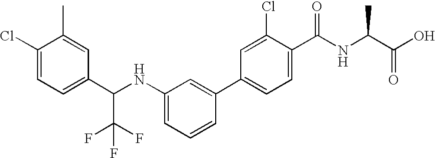

The title compound was prepared following a procedure analogous to steps 3, 4 and 5 of Example EX68 using intermediate INT30 and 2-chloro-4-(4,4,5,5-tetramethyl-[1,3,2]dioxaborolan-2-yl)-benzoic acid methyl ester in step 3 (followed by LiOH-induced ester hydrolysis), and alanine methyl ester hydrochloride in step 4.

LC/MS Method 2: MS (ESI): 525 [M+H]⁺, rt=3.33 min.

### Example EX95

### 1-({3-Chloro-3′-[1-(4-chloro-3-methyl-phenyl)-2,2,2-trifluoro-ethylamino]-biphenyl-4-carbonyl}-amino)-cyclopropanecarboxylic acid

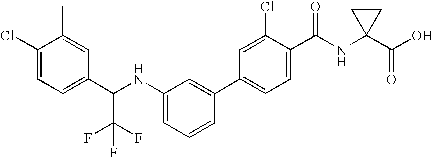

The title compound was prepared following a procedure analogous to steps 3, 4 and 5 of Example EX68 using intermediate INT30 and 2-chloro-4-(4,4,5,5-tetramethyl-[1,3,2]dioxaborolan-2-yl)-benzoic acid methyl ester in step 3 (followed by LiOH-induced ester hydrolysis), and 1-amino-cyclopropanecarboxylic acid ethyl ester hydrochloride in step 4.

LC/MS Method 2: MS (ESI): 537 [M+H]⁺, rt=3.35 min.

### Example EX96

### (S)-2-({3-Chloro-3′-[(S)-1-(4-chloro-3-methyl-phenyl)-2,2,2-trifluoro-ethylamino]-biphenyl-4-carbonyl}-methyl-amino)-propionic acid

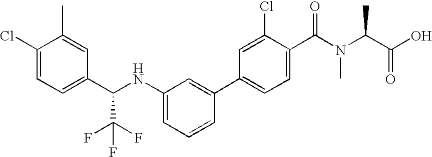

The title compound was prepared following a procedure analogous to steps 3, 4 and 5 of Example EX68 using intermediate INT30 and 2-chloro-4-(4,4,5,5-tetramethyl-[1,3,2]dioxaborolan-2-yl)-benzoic acid methyl ester in step 3 (followed by LiOH-induced ester hydrolysis), and N-methyl alanine methyl ester hydrochloride in step 4. The optically pure title product was obtained by preparative chiral separation (Method A) using n-heptane/iso-propanol (85:15)+0.1% TFA. Chiral HPLC method C: rt=17.6 min, n-heptane/iso-propanol (85:15)+0.1% TFA.

LC/MS Method 2: MS (ESI): 539 [M+H]⁺, rt=3.46 min.

### Example EX97

### (S)-2-({3-Chloro-3′-[1-(4-chloro-3-methyl-phenyl)-1-methyl-ethylamino]-biphenyl-4-carbonyl}-amino)-propionic acid

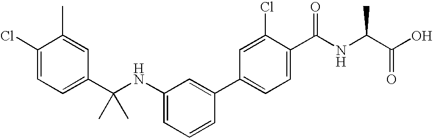

The title compound was prepared following a procedure analogous to steps 3, 4 and 5 of Example EX68 using intermediate INT43 and 2-chloro-4-(4,4,5,5-tetramethyl-[1,3,2]dioxaborolan-2-yl)-benzoic acid methyl ester in step 3 (followed by LiOH-induced ester hydrolysis), and alanine methyl ester hydrochloride in step 4.

LC/MS Method 2: MS (ESI): 485 [M+H]⁺, rt=2.89 min.

### Example EX98

### (S)-2-({3-Chloro-3′-[1-(4-chloro-3-methyl-phenyl)-1-methyl-ethylamino]-biphenyl-4-carbonyl}-methyl-amino)-propionic acid

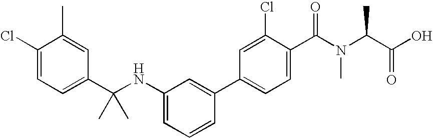

The title compound was prepared following a procedure analogous to steps 3, 4 and 5 of Example EX68 using intermediate INT43 and 2-chloro-4-(4,4,5,5-tetramethyl-[1,3,2]dioxaborolan-2-yl)-benzoic acid methyl ester in step 3 (followed by LiOH-induced ester hydrolysis), and N-methyl alanine methyl ester hydrochloride in step 4.

LC/MS Method 2: MS (ESI): 499 [M+H]⁺, rt=3.01 min.

### Example EX99

### 1-({3′-[(R)-1-(4-Chloro-3,5-dimethyl-phenyl)-ethylamino]-3,5-dimethyl-biphenyl-4-carbonyl}-amino)-cyclopropanecarboxylic acid

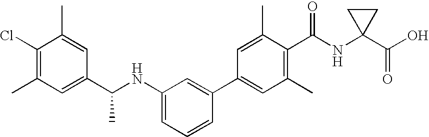

### (1) (3-Bromo-phenyl)-[1-(4-chloro-3,5-dimethyl-phenyl)-ethyl]-amine, INT46

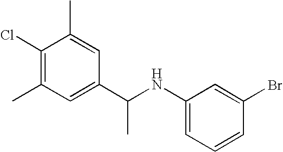

To a solution of 1-(4-chloro-3,5-dimethylphenyl)ethanone (420 mg, 2.3 mmol) and 3-bromaniline (0.238 ml, 2.185 mmol) in 20 ml of methanol was added decaborane (169 mg, 1.38 mmol) and the resulting mixture was stirred at room temperature overnight. The mixture was concentrated and purified by chromatography on silica gel using cyclohexane and ethylacetate (from 0% to 10%) to give the title compound as white solid.

### (2) (3-Bromo-phenyl)-[(R)-1-(4-chloro-3,5-dimethyl-phenyl)-ethyl]-amine, INT47

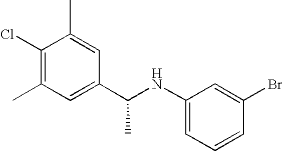

The title compound was obtained by preparative chiral separation of intermediate INT46 (Method B). Chiral HPLC method E: rt=6.64 min, n-heptane/EtOH/MeOH (80:10:10). MS (ESI): 336-338 [M−H]⁻, ¹H-NMR (CDCl₃): δ (ppm) 7.04 (s, 2H), 6.93 (t, 1H), 6.76 (d, 1H), 6.66 (s, 1H), 6.38 (d, 1H), 4.34 (q, 1H), 4.03 (br s, 1H), 2.35 (s, 6H), 1.46 (d, 3H).

### (3) 3′-[(R)-1-(4-Chloro-3,5-dimethyl-phenyl)-ethylamino]-3,5-dimethyl-biphenyl-4-carboxylic acid hydrochloride, INT48

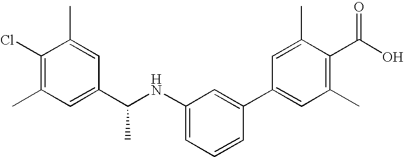

A solution of intermediate INT47 (210 mg, 0.620 mmol) and intermediate INT23 (171 mg, 0.620 mmol) in DME (10 ml) was treated with 10% sodium bicarbonate solution (3.65 ml, 4.34 mmol). The mixture was stirred under argon for 10 minutes at room temperature after which Pd(PPh₃)₄ (14.33 mg, 0.012 mmol) was added. The mixture was heated at 85° C. for 3 hours. It was then cooled to room temperature, filtered through Celite and most of the solvents were evaporated. The pH of the residue was adjusted to about 2 with 0.5N-HCl and the mixture was extracted twice with ethyl acetate. The organic extracts were dried over sodium sulfate, filtered and evaporated. The crude was dissolved in DCM and treated with an excess of ethereal HCl. The precipitate was quickly filtered off (slightly hydroscopic) and dried under high vacuum. The crude was used without further purification.

UPLC: rt=2.38 min. MS (ESI): 408 [M+H]⁺.

### (4) 1-({3′-[(R)-1-(4-Chloro-3,5-dimethyl-phenyl)-ethylamino]-3,5-dimethyl-biphenyl-4-carbonyl}-amino)-cyclopropanecarboxylic acid ethyl ester, INT49

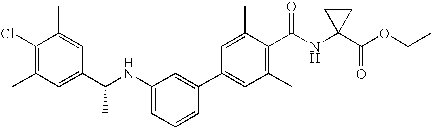

A solution of the hydrochloride INT48 (99 mg, 0.223 mmol) in 1 ml of DCM and one drop of DMF was treated with thionyl chloride (33 μl, 0.446 mmol). The solution was stirred at 50° C. for 30 minutes, then evaporated and dried under high vacuum for 10 minutes. The crude foam was then dissolved in 2 ml of THF and 1-amino-cyclopropyl-1-carboxylic acid ethyl ester hydrochloride (40.6 mg, 0.245 mmol) and DIEA (0.117 ml, 0.668 mmol) were added sequentially. This mixture was stirred at room temperature for 2 hours. The solvents were then evaporated and the crude was dissolved in 20 ml of ethyl acetate. The organic layer was washed with 0.2N-HCl, 10% sodium carbonate and brine, dried over sodium sulfate, filtered and evaporated. The crude was purified by chromatography on silica gel using cyclohexane and ethyl acetate (from 0% to 15%).

UPLC: rt=2.52 min. MS (ESI): 519 [M+H]⁺, ¹H-NMR (CDC)-d₃): δ (ppm) 7.13 (t, 1H), 7.09 (br s, 4H), 6.83 (d, 1H), 6.68 (s, 1H), 6.48 (d, 1H), 6.15 (s, 1H), 4.41 (m, 1H), 4.22 (q, 2H), 2.38 (s, 6H), 2.36 (s, 6H), 1.69 (m, 2H), 1.50 (m, 2H), 1.22-1.32 (m, 6H).

### (5) 1-({3′-[(R)-1-(4-Chloro-3,5-dimethyl-phenyl)-ethylamino]-3,5-dimethyl-biphenyl-4-carbonyl}-amino)-cyclopropanecarboxylic acid

The ester INT49 (50 mg, 0.096 mmol) was dissolved in 4 ml of THF and treated with 1N-LiOH (0.385 ml, 0.385 mmol). The mixture was stirred over night at 65° C. Most of the solvent was evaporated. The crude was treated with water (5 ml), acidified with 0.2M-HCl (3 ml) and extracted twice with ethyl acetate (10 ml). The organic extracts were combined, dried over sodium sulfate, filtered and evaporated to give the desired compound.

LC/MS Method 2: MS (ESI): 491 [M+H]⁺, rt=3.01 min. ¹H-NMR (DMSO-d₆): δ (ppm) 12.4 (v br s, 1H), 8.77 (br s, 1H), 7.21 (s, 2H), 7.08 (s, 2H), 7.03 (t, 1H), 6.7 (m, 2H), 6.44 (d, 1H), 6.22 (d, 1H), 4.43 (m, 1H), 2.29 (s, 6H), 2.25 (s, 6H), 1.38 (d, 3H), 1.02-1.28 (m, 4H).

### Example EX100

### (S)-2-({3′-[(R)-1-(4-Chloro-3,5-dimethyl-phenyl)-ethylamino]-3,5-dimethyl-biphenyl-4-carbonyl}-amino)-propionic acid

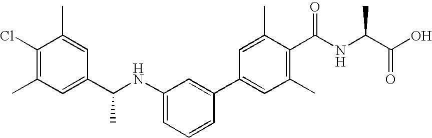

The title compound was prepared from intermediate INT48 following a procedure analogous to Example EX99 using alanine methyl ester hydrochloride in step 4.

LC/MS Method 2: MS (ESI): 491 [M+H]⁺, rt=3.01 min. ¹H-NMR (DMSO-d₆): δ (ppm) 12.46 (v br s, 1H), 8.59 (d, 1H), 7.23 (s, 2H), 7.11 (s, 2H), 7.05 (t, 1H), 6.74 (m, 2H), 6.47 (d, 1H), 6.21 (d, 1H), 4.42 (m, 2H), 2.30 (s, 3H), 2.27 (s, 6H), 1.41 (d, 3H), 1.33 (d, 3H).

### Example EX101

### (S)-2-({3′-[(R)-1-(4-Chloro-3,5-dimethyl-phenyl)-propylamino]-3,5-dimethyl-biphenyl-4-carbonyl}-amino)-propionic acid

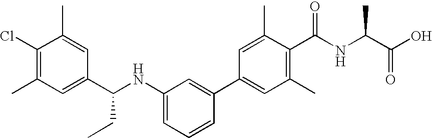

The title compound was prepared from intermediate INT52 (synthesis described below) following a procedure analogous to Example EX99 using alanine methyl ester hydrochloride in step 4.

LC/MS Method 2: MS (ESI): 491 [M+H]⁺, rt=3.01 min. ¹H-NMR (DMSO-d₆): δ (ppm) 12.46 (v br s, 1H), 8.59 (d, 1H), 7.23 (s, 2H), 7.11 (s, 2H), 7.05 (t, 1H), 6.74 (m, 2H), 6.47 (d, 1H), 6.21 (d, 1H), 4.42 (m, 2H), 2.30 (s, 3H), 2.27 (s, 6H), 1.41 (d, 3H), 1.33 (d, 3H).

LC/MS Method 2: MS (ESI): 493 [M+H]⁺, rt=3.14 min.

### (1) 1-(4-Chloro-3,5-dimethyl-phenyl)-propan-1-one, INT50

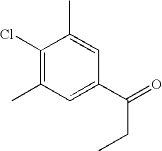

EtMgBr (3M in diethyl ether, 3.20 ml, 9.60 mmol) was slowly added to a solution of 4-chloro-3,5-dimethyl-benzonitrile (795 mg, 4.80 mmol) in 20 ml of benzene at room temperature. The mixture was then heated under reflux for 3 hours, cooled in an ice-bath and carefully treated with 6N-HCl (7.68 ml, 46.1 mmol). This mixture was heated again under reflux for 2 hours. It was then allowed to cool to room temperature and extracted with ethyl acetate. The organic layer was washed with brine, dried over sodium sulfate, filtered and evaporated to give the title compound as a beige powder.

MS (ESI): 196.9 [M+H]⁺, ¹H-NMR (CDCl₃): δ (ppm) 7.67 (s, 2H), 2.96 (q, 2H), 2.43 (s, 6H), 1.21 (t, 3H).

### (2) (3-Bromo-phenyl)-[1-(4-chloro-3,5-dimethyl-phenyl)-propyl]-amine, INT51

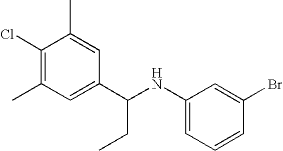

To a solution of ketone INT50 (910 mg, 4.63 mmol) and 3-bromaniline (0.504 ml, 4.63 mmol) in 25 ml of methanol was added decaborane (283 mg, 2.313 mmol) and the resulting mixture was stirred at room temperature overnight. The mixture was concentrated and purified by chromatography on silica gel using cyclohexane and ethylacetate (from 0% to 2%) to give the title compound as white solid.

### (3) (3-Bromo-phenyl)-[(R)-1-(4-chloro-3,5-dimethyl-phenyl)-propyl]-amine, INT52

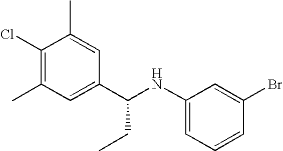

The title compound was obtained by preparative chiral separation of intermediate INT51 (Method B). Chiral HPLC method E: rt=6.28 min, n-heptane/EtOH/MeOH (80:10:10).

MS (ESI): 350-352 [M−H], ¹H-NMR (CDCl₃): δ (ppm) 7.0 (s, 2H), 6.91 (t, 1H), 6.74 (d, 1H), 6.66 (s, 1H), 6.38 (d, 1H), 4.07 (br m, 2H), 2.35 (s, 6H), 1.76 (m, 2H), 0.93 (t, 3H).

### Example EX102

### 1-({3-[(R)-1-(4-Chloro-3,5-dimethyl-phenyl)-propylamino]-3,5-dimethyl-biphenyl-4-carbonyl}-amino)-cyclopropanecarboxylic acid

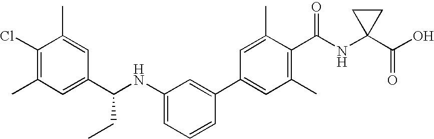

The title compound was prepared from intermediate INT52 following a procedure analogous to Example EX99.

LC/MS Method 2: MS (ESI): 505 [M+H]⁺, rt=3.17 min.

### Example EX103

### (S)-2-({3′-[1-(4-Chloro-3-methyl-phenyl)-2,2-difluoro-ethylamino]-3,5-dimethyl-biphenyl-4-carbonyl}-methyl-amino)-propionic acid

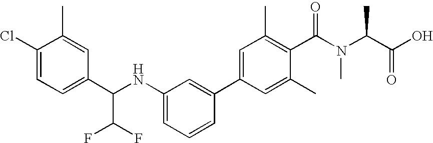

The title compound was prepared from INT31 following the steps 3, 4 and 5 of the preparation of Example EX68 using N-methylalanine methyl ester hydrochloride and HATU as coupling reagent in step 3.

LC/MS Method 2: Two rotamers were observed: MS (ESI): 515 [M+H]⁺, rt=2.84+2.97 min.

### Example EX104

### (S)-2-[(3′-{[(R)-1-(4-Chloro-3-methyl-phenyl)-propyl]-methyl-amino}-3,5-dimethyl-biphenyl-4-carbonyl)-amino]-propionic acid

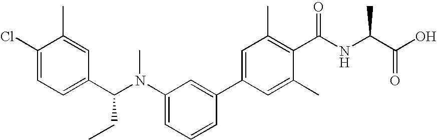

The title compound was prepared from INT54 (synthesis described below) following the steps 3, 4 and 5 of the preparation of Example EX99 using alanine methyl ester in step 4.

LC/MS Method 2: MS (ESI): 493 [M+H]⁺, rt=3.27 min.

### (1) (3-Bromo-phenyl)-[(R)-1-(4-chloro-3-methyl-phenyl)-propyl]-amine, INT53

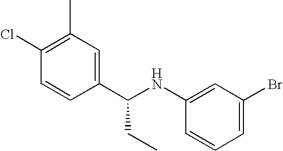

The title compound was prepared in a similar manner to INT26 using ketone INT8 and toluene as the solvent for imine formation.

¹H-NMR (CDCl₃): δ (ppm) 7.27 (d, 1H), 7.16 (d, 1H), 7.06 (dd, 1H), 6.91 (t, 1H), 6.76-6.73 (m, 1H), 6.65 (t, 1H), 6.39-6.35 (m, 1H), 4.15-4.05 (m, 2H), 2.35 (s, 3H), 1.85-1.70 (m, 2H), 0.93 (t, 3H). LC/MS Method 1: MS (ESI): 339-343 [M+H]⁺, rt=1.83 min.

### (2) (3-Bromo-phenyl)-[(R)-1-(4-chloro-3-methyl-phenyl)-propyl]-methyl-amine, INT54

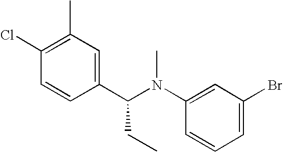

A solution of INT53 (8.47 g, 25.0 mmol) in methanol (125 ml) was cooled to 0° C. before addition under an argon atmosphere of formaldehyde (37% in water, 2.79 ml, 37.5 mmol) and decaborane (1.528 g, 12.50 mmol). The reaction mixture was stirred overnight. The solvent was removed under reduced pressure and the residue dissolved in 15 mL EtOAc. 10 mL 1N HCl was added and the mixture was vigorously stirred at rt for 30 min. Then it was cooled down to 0 C and 15 mL 1N NaOH was added and stirring was continued for another 30 min at rt. The organic layer was separated and the aqueous layer further extracted with EtOAc. The combined organic layers were washed with brine, dried over sodium sulfate, filtered and concentrated. The crude was purified by chromatography on silica gel using cyclohexane and ethyl acetate (99:1) to give INT54 as a colorless oil.

¹H-NMR (CDCl₃): δ (ppm) 7.26 (d, 1H), 7.09-7.03 (m, 2H), 6.99 (dd, 1H), 6.92 (t, 1H), 6.83-6.80 (m, 1H), 6.72 (dd, 1H) 4.76-4.72 (m, 2H), 2.67 (s, 3H), 2.34 (s, 3H), 2.11-1.87 (m, 2H), 0.96 (t, 3H). LC/MS Method 1: MS (ESI): 352-356 [M+H]⁺, rt=1.93 min.

### Example EX105

### (S)-2-[(3′-{[(R)-1-(4-Chloro-3-methyl-phenyl)-propyl]-methyl-amino}-3,5-dimethyl-biphenyl-4-carbonyl)-methyl-amino]-propionic acid

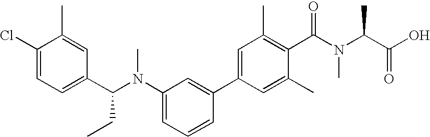

The title compound was prepared from INT54 following the steps 3, 4 and 5 of the preparation of Example EX99 using N-methylalanine methyl ester in step 4.

LC/MS Method 1: Two rotamers were observed: MS (ESI): 507 [M+H]⁺, rt=1.57+1.66 min.

### Example EX106

### (S)-2-({3′-[(S)-1-(4-Chloro-3-methyl-phenyl)-2,2,2-trifluoro-ethylamino]-3,5-dimethyl-biphenyl-4-carbonyl}-amino)-propionic acid

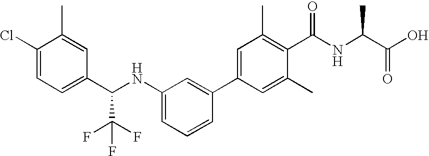

The title compound was prepared in a similar manner to Example EX74 with an additional chiral separation step before saponification of the ester.

LC/MS Method 2: MS (ESI): 519 [M+H]⁺, rt=2.85 min.

### Example EX107

### (S)-2-({3′-[(R)-1-(4-Chloro-3-methyl-phenyl)-propylamino]-3,5-dimethyl-biphenyl-4-carbonyl}-methyl-amino)-propionic acid

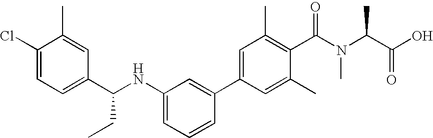

The title compound was prepared from INT53 following the steps 3, 4 and 5 of the preparation of Example EX99 using N-methylalanine methyl ester in step 4.

LC/MS Method 1: Two rotamers were observed: MS (ESI): 493 [M+H]⁺, rt=1.59+1.65 min.

### Example EX108

### 1-[(3′-{[(R)-1-(4-Chloro-3-methyl-phenyl)-propyl]-methyl-amino}-3,5-dimethyl-biphenyl-4-carbonyl)-amino]-cyclopropanecarboxylic acid

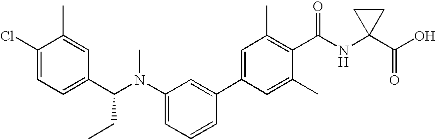

The title compound was prepared following the steps 4 and 5 of the preparation of Example EX81 using INT54 and INT55 (synthesis described below) in step 4.

LC/MS Method 1: MS (ESI): 505 [M+H]⁺, rt=1.55 min.

### (1) 1-[2,6-Dimethyl-4-(4,4,5,5-tetramethyl-[1,3,2]dioxaborolan-2-yl)-benzoylamino]-cyclopropanecarboxylic acid ethyl ester, INT55

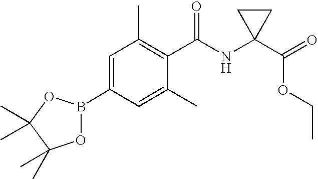

Argon was bubbled for 15 min through a mixture of bis(pinacoloto)diboron (5.43 g, 21.16 mmol), INT36 (6 g, 17.64 mmol) and potassium acetate (10.49 g, 106 mmol) in dioxane (202 mL). Then PdCl₂(dppf) (720 mg, 0.882 mmol) was added and the resulting mixture was stirred at 80° C. overnight. After cooling down, the mixture was concentrated and partitioned between ethyl acetate and 5% aqueous sodium bicarbonate. The organic layer was further washed with brine, dried over sodium sulfate, filtered and concentrated. The residue was purified by chromatography on silica gel (cyclohexane/EtOAc) to give INT55 as a white solid.

¹H-NMR (DMSO-d₆): δ (ppm) 8.90 (s, 1H), 7.32 (s, 2H), 4.10 (q, 2H), 2.23 (s, 6H), 1.42 (m, 2H), 1.27 (s, 12H), 1.19 (t, 3H), 1.09 (m, 2H).

### Example EX109

### 1-({3′-[(R)-1-(4-Chloro-3-methyl-phenyl)-propylamino]-3,5-dimethyl-biphenyl-4-carbonyl}-amino)-cyclopropanecarboxylic

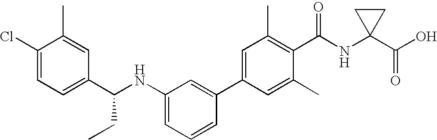

The title compound was prepared in a similar manner to Example EX27 with an additional chiral separation step before saponification of the ester.

LC/MS Method 2: MS (ESI): 491 [M+H]⁺, rt=2.95 min.

### Example EX110

### 1-[(3′-{[(R)-1-(4-Chloro-3-methyl-phenyl)-propyl]-methyl-amino}-3,5-dimethyl-biphenyl-4-carbonyl)-methyl-amino]-cyclopropanecarboxylic acid

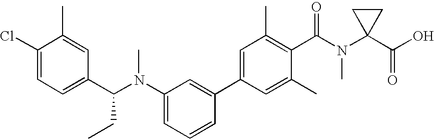

The title compound was prepared following the steps 4 and 5 of the preparation of Example EX81 using INT54 and INT38 in step 4.

LC/MS Method 1: MS (ESI): 519 [M+H]⁺, rt=1.66 min.

### Example EX111

### 1-({3′-[(R)-1-(4-Chloro-3-methyl-phenyl)-propylamino]-3,5-dimethyl-biphenyl-4-carbonyl}-methyl-amino)-cyclopropanecarboxylic acid

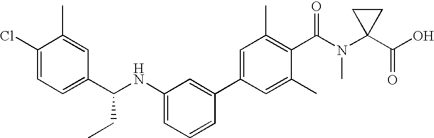

The title compound was prepared following the steps 4 and 5 of the preparation of Example EX81 using INT53 and INT38 in step 4.

LC/MS Method 1: MS (ESI): 519 [M+H]⁺, rt=1.64 min.

### Example EX112

### 1-({3′-[(S)-1-(4-Chloro-3-methyl-phenyl)-2,2,2-trifluoro-ethylamino]-3,5-dimethyl-biphenyl-4-carbonyl}-amino)-cyclopropanecarboxylic acid

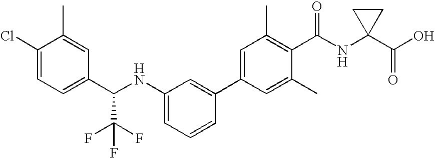

The title compound was prepared following the steps 4 and 5 of the preparation of Example EX81 using INT55 and INT38 in step 4 and with an additional chiral separation step before saponification of the ester.

LC/MS Method 2: MS (ESI): 531 [M+H]⁺, rt=2.96 min.

### Example EX113

### (S)-2-({3′-[(R)-1-(4-Chloro-3-methyl-phenyl)-propylamino]-3,5-dimethyl-biphenyl-4-carbonyl}-methyl-amino)-propionic acid

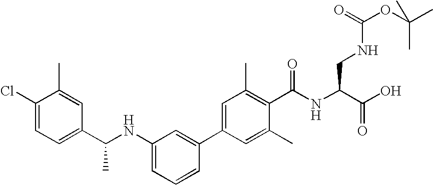

The title compound can be prepared in a similar manner to Example EX73 using (5)-methyl 2-amino-3-(tert-butoxycarbonylamino)propanoate in step 3.

LC/MS Method 2: MS (ESI): 580 [M+H]⁺, rt=3.11 min.

### Example EX114

### (S)-3-Amino-2-({3′-[(R)-1-(4-chloro-3-methyl-phenyl)-ethylamino]-3,5-dimethyl-biphenyl-4-carbonyl}-amino)-propionic acid

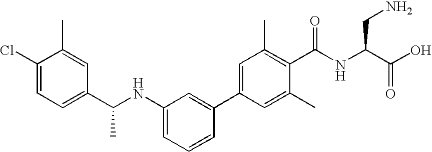

The title compound was prepared by HCl induced Boc deprotection of EX113. LC/MS Method 2: MS (ESI): 480 [M+H]⁺, rt=2.29 min.

### Example EX115

### 1-({5′-[(R)-1-(4-Chloro-3-methyl-phenyl)-propylamino]-2′-fluoro-3,5-dimethyl-biphenyl-4-carbonyl}-amino)-cyclopropanecarboxylic acid

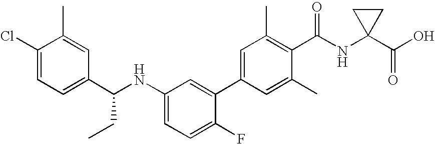

The title compound was prepared in a similar manner to EX25 with an additional chiral separation step before saponification of the ester.

LC/MS Method 2: MS (ESI): 509 [M+H]⁺, rt=3.54 min.

### Example EX116

### (S)-2-[(5′-{[(R)-1-(4-Chloro-3-methyl-phenyl)-propyl]-methyl-amino}-2′-fluoro-3,5-dimethyl-biphenyl-4-carbonyl)-amino]-propionic acid

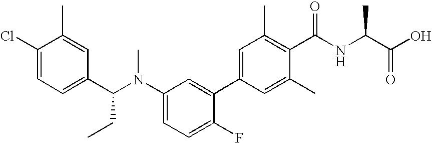

The title compound was prepared from INT57 (synthesis described below) following the steps 3, 4 and 5 of the preparation of Example EX99 using alanine methyl ester in step 4.

LC/MS Method 2: MS (ESI): 511 [M+H]⁺, rt=3.30 min.

### (1) (3-Bromo-4-fluoro-phenyl)-[(R)-1-(4-chloro-3-methyl-phenyl)-propyl]-amine, INT56

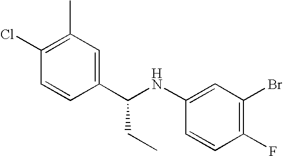

The title compound was prepared in a similar manner to INT26 using ketone INT8, 3-bromo-4-fluoroaniline and toluene as the solvent for the imine formation.

¹H-NMR (DMSO-d₆): δ (ppm) 7.32 (d, 1H), 7.31 (s, 1H), 7.19-7.14 (m, 1H), 6.98 (t, 6.76-6.72 (m, 1H), 6.49-6.45 (m, 1H), 4.17 (t, 1H), 2.28 (s, 3H), 1.77-1.69 (m, 1H), 1.66-1.58 (m, 1H), 0.86 (t, 3H). LC/MS Method 2: MS (ESI): 356-360 [M+H]⁺, rt=3.54 min.

### (2) (3-Bromo-4-fluoro-phenyl)-[(R)-1-(4-chloro-3-methyl-phenyl)-propyl]-methyl-amine, INT57

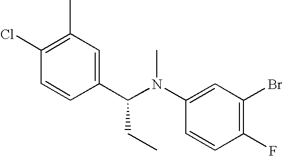

A solution of INT56 (1.4 g, 3.93 mmol) in methanol (20 ml) was cooled to 0° C. before addition under an argon atmosphere of formaldehyde (37% in water, 0.438 ml, 5.89 mmol) and decaborane (240 mg, 1.96 mmol). The reaction mixture was stirred overnight. The solvent was removed under reduced pressure and the residue dissolved in 15 mL EtOAc. 10 mL 1N HCl was added and the mixture was vigorously stirred at it for 30 min. Then it was cooled down to 0 C and 15 mL 1N NaOH was added and stirring was continued for another 30 min at rt. The organic layer was separated and the aqueous layer further extracted with EtOAc. The combined organic layers were washed with brine, dried over sodium sulfate, filtered and concentrated. The crude was purified by chromatography on silica gel using cyclohexane and ethyl acetate (99:1).

¹H-NMR (DMSO-d₆): δ (ppm) 7.32 (d, 1H), 7.23 (s, 1H), 7.14 (t, 1H), 7.06 (dd, 1H), 699-6.97 (m, 1H), 6.86-6.82 (m, 1H), 4.83-4.80 (m, 1H), 2.65 (s, 1H), 2.29 (s, 3H), 2.00-1.86 (m, 0.86 (t, 3H). LC/MS Method 2: MS (ESI): 370-374 [M+H]⁺, rt=3.89 min.

### Example EX117

### (S)-2-({5′-[(R)-1-(4-Chloro-3-methyl-phenyl)-propylamino]-2′-fluoro-3,5-dimethyl-biphenyl-4-carbonyl}-methyl-amino)-propionic acid

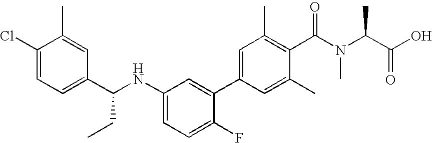

The title compound was prepared from INT56 following the steps 3, 4 and 5 of the preparation of Example EX99 using N-methylalanine methyl ester in step 4.

LC/MS Method 2: Two rotamers were observed: MS (ESI): 511 [M+H]⁺, rt=3.05+3.18 min.

### Example EX118

### (S)-2-[(5′-{[(R)-1-(4-Chloro-3-methyl-phenyl)-propyl]-methyl-amino}-2′-fluoro-3,5-dimethyl-biphenyl-4-carbonyl)-methyl-amino]-propionic acid

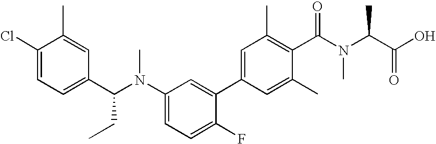

The title compound was prepared from INT57 following the steps 3, 4 and 5 of the preparation of Example EX99 using N-methylalanine methyl ester in step 4.

LC/MS Method 2: Two rotamers were observed: MS (ESI): 525 [M+H]⁺, rt=3.33+3.49 min.

The compounds of formula (I) in free form or in pharmaceutically acceptable salt form, exhibit valuable pharmacological properties, e.g. as S1P1 receptor antagonists, e.g. as indicated in in vitro and in vivo tests and are therefore indicated for therapy.

A. In vitro

The compounds of formula (I) have typically binding affinity to human SIP receptors as determined in following assay:

### Human S1P1 Receptor Calcium FLIPR Antagonist Assay

### (HeLa Gα16 S1P1)

The assay measures intracellular changes of Ca²⁺ mediated by the synthetic probing agonist 3-{[2-(2-Trifluoromethyl-biphenyl-4-yl)-benzo[b]thiophen-5-ylmethyl]amino}-propionic acid (GNF-AC-1) in the HeLa-S1P1/Gα16 cell clone 1: HeLa (human cervix carcinoma, ATCC CCL2) cells stably expressing N-terminally myc-tagged human S1P1 receptors (GenBank™ accession No. NM 001400; UNIPROT P21453) and promiscuous Gal 6 protein (GenBank™accession number M63904, Swissprot P30679) are cultured at 37° C., 5% CO₂, and 95 relative humidity. The cells are plated in 384 well black plates (10′000 cells per well). After 24 hours the cells are loaded with Fluo-4-AM (1.6 μM in HBSS and 2.5 mM probenicid) for 1 hour at 37° C. After washing, the cells are transferred to the FLIPR. The test compounds are added at different concentrations 0100 μM) in HBSS in the presence of 0.1% BSA and changes in fluorescence are recorded (indication of agonism). The probing agonist is added 20-30 minutes afterwards to the wells at a concentration giving 80% of the maximal activity (EC₈₀). After each addition, time points are collected as follows: 20 time points (2 seconds) before the addition of the agonist (Fmin) and 60 time points (1 or 2 seconds) after the addition of the probing agonist. This allows the determination of the maximal fluorescence (Fmax). The ratio (Fmax-Fmin)/Fmin is plotted against the log of the concentration of the test compounds and the IC₅₀ (relative antagonism) is calculated using the XLfit-4 software. Compounds with an inhibition <20% are usually considered “inactive”. A concentration-response curve of the probing agonist is determined on each plate in parallel. The IC₅₀ values of compounds of formula (I) in the above described Human SIP Receptor Calcium FLIPR Antagonist Assay are displayed in Table 1.

TABLE 1

| Example No. | IC₅₀ [nM] |
|---|---|
| 1 | 2 |
| 2 | 5 |
| 3 | 2 |
| 4 | 1 |
| 5 | 2 |
| 6 | 2 |
| 7 | 2 |
| 8 | 2 |
| 9 | 3 |
| 10 | 2 |
| 11 | 4 |
| 12 | 3 |
| 13 | 5 |
| 14 | 4 |
| 15 | 2 |
| 16 | 2 |
| 17 | 5 |
| 18 | 4 |
| 19 | 2 |
| 20 | 8 |
| 21 | 2 |
| 22 | 2 |
| 23 | 1 |
| 24 | 1 |
| 25 | 2 |
| 26 | 1 |
| 27 | 2 |
| 28 | 3 |
| 29 | 2 |
| 30 | 2 |
| 33 | 1 |
| 34 | 3 |
| 35 | 8 |
| 36 | 2 |
| 37 | 2 |
| 40 | 2 |
| 42 | 3 |
| 43 | 3 |
| 45 | 5 |
| 46 | 2 |
| 47 | 2 |
| 49 | 3 |
| 50 | 3 |
| 51 | 6 |
| 52 | 1 |
| 53 | 5 |
| 55 | 9 |
| 56 | 1 |
| 57 | 6 |
| 58 | 2 |
| 59 | 3 |
| 60 | 1 |
| 61 | 1 |
| 62 | 1 |
| 63 | 2 |
| 64 | 1 |
| 65 | 6 |
| 66 | 3 |
| 67 | 3 |
| 68 | 1 |
| 69 | 2 |
| 70 | 2 |
| 72 | 2 |
| 73 | 4 |
| 74 | 8 |
| 75 | 6 |
| 76 | 3 |
| 78 | 7 |
| 79 | 2 |
| 80 | 4 |
| 81 | 1 |
| 82 | 3 |
| 83 | 2 |
| 84 | 2 |
| 85 | 11 |
| 86 | 6 |
| 87 | 4 |
| 88 | 5 |
| 89 | 13 |
| 90 | 10 |
| 91 | 4 |
| 92 | 4 |
| 93 | 4 |
| 94 | 14 |
| 95 | 14 |
| 96 | 2 |
| 97 | 1 |
| 98 | 4 |
| 99 | 1 |
| 100 | 1 |
| 101 | 1 |
| 102 | 1 |
| 103 | 3 |
| 104 | 1 |
| 105 | 10 |
| 106 | 2 |
| 107 | 1 |
| 108 | 8 |
| 109 | 2 |
| 110 | 35 |
| 111 | 8 |
| 112 | 2 |
| 113 | 2 |
| 114 | 1 |
| 115 | 2 |
| 116 | 2 |
| 117 | 2 |
| 118 | 4 |

B. In vivo

The compounds of formula (I) typically induce the depletion of blood lymphocyte as may be determined in the assay described below. Moreover, compounds of formula (I) are typically efficacious in the said assay also when administered via the per oral route of administration.

### Measurement of Circulating Lymphocytes:

The test compounds (or salts thereof) are dissolved in a vehicle such as water, saline, PEG (polyethylene glycol) 200, or PBS (phosphate buffered saline). Rats (Lewis strain, male, 6-12 weeks old) are administered up to 100 mg/kg of the test compounds in 2 ml/kg vehicle via per oral or subcutaneous application. The vehicle or a reference salt and FTY720 (0.3 mg/kg) are included as negative and positive controls, respectively.

Blood is collected from the sublingual vein before and 2, 8 and 24 hours or 14, 18 and 24 hours after the test compound administration under short isoflurane anesthesia. Whole blood samples are subjected to hematology analysis. Peripheral lymphocyte counts are determined using an automated analyzer. The Haemathology System uses a combination of light scatter, cytochemical staining and nuclear density on two independent channels to measure the total and differential white cell counts. Two to four rats are used to assess the lymphocyte depletion activity of each compound screened. The data are presented as mean±SEM.

As an example, Table 2 shows the effect on lymphocyte counts 14 hours after oral administration of 30 mg/kg of some compounds of formula (I) to male Lewis rats as compared to a group of animals treated with vehicle only.

TABLE 2

| | Residual Lymphocyte Counts |
|---|---|
| Example No. | 14 hours after 30 mg/kg p.o. dosing |
| 3 | 15.5% ± 1.3 |
| 6 | 15.9% ± 1.2 |
| 7 | 14.8% ± 0.8 |
| 8 | 16.7% ± 0.6 |
| 20 | 18.7% ± 1.9 |
| 22 | 18.7% ± 0.2 |
| 23 | 17.4% ± 0.9 |
| 24 | 16.8% ± 1.9 |
| 25 | 15% ± 0.5 |
| 26 | 12.9% ± 0.8 |
| 73 | 16.6% ± 0.5 |
| 74 | 15.5% ± 1.1 |
| 75 | 19.4% ± 0.2 |
| 81 | 15.4% ± 0.0 |
| 82 | 10.8% ± 1.2 |
| 83 | 15.2% ± 1.1 |
| 84 | 18.6% ± 2.4 |
| 85 | 14.7% ± 1.3 |
| 99 | 15.5% ± 3.1 |
| 100 | 20.9% ± 0.1 |
| 103 | 13.9% ± 2.3 |
| 105 | 20.9% ± 4.0 |
| 106 | 10.9% ± 1.9 |
| 108 | 19.6% ± 2.3 |
| 109 | 19.1% ± 0.7 |
| 112 | 18.3% ± 1.1 |
| 115 | 15.8% ± 2.7 |
| 117 | 15.0% ± 3.0 |

The compounds of formula (I) are, therefore, useful in the treatment and/or prevention of diseases or disorders mediated by lymphocytes interactions, e.g. in transplantation, such as acute or chronic rejection of cell, tissue or organ allo- or xenografts or delayed graft function, graft versus host disease, autoimmune diseases, e.g. rheumatoid arthritis, systemic lupus erythematosus, hashimoto's thyroidis, multiple sclerosis, myasthenia gravis, neuropathic pain, Behcet's disease, Wegener's granulamatosis, ankylosing spondylitis, polymyositis, CIDP (Chronic Idiopathic Demyelinating Polyneuropathy), diabetes type I or II and the disorders associated therewith, vasculitis, pernicious anemia, Sjoegren syndrome, uveitis, psoriasis, Graves opthalmopathy, alopecia greata and others, allergic diseases, e.g. allergic asthma, atopic dermatitis, allergic rhinitis/conjunctivitis, allergic contact dermatitis, inflammatory diseases optionally with underlying aberrant reactions, e.g. inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, ischemia/reperfusion injury, e.g. myocardial infarction, stroke, gut ischemia, renal failure or hemorrhage shock, traumatic shock, cancer, e.g. breast cancer, T cell lymphomas or T cell leukemias, infectious diseases, e.g. toxic shock (e.g. superantigen induced), septic shock, adult respiratory distress syndrome or viral infections, e.g. AIDS, viral hepatitis, e.g. hepatitis B or C, chronic bacterial infection, or neurodegenerative diseases, e.g. Alzheimer disease, amyotrophic lateral sclerosis or senile dementia. Examples of cell, tissue or solid organ transplants include e.g. pancreatic islets, stem cells, bone marrow, corneal tissue, neuronal tissue, heart, lung, combined heart-lung, kidney, liver, bowel, pancreas, trachea or oesophagus. Furthermore, the compounds of formula (I) are useful in the treatment and/or prevention of diseases or disorders associated with deregulated angiogenesis for example diseases caused by ocular neovascularisation, especially retinopathies (diabetic retinopathy, age-related macular degeneration); psoriasis; haemangioblastomas, such as “strawberry-marks” (=haemangioma); various inflammatory diseases, such as arthritis, especially rheumatoid arthritis, arterial atherosclerosis and atherosclerosis occurring after transplants, endometriosis or chronic asthma; and, especially, tumor diseases (solid tumors, but also leukemias and other liquid tumors).

The present invention further provides:
1.1 A method for preventing or treating acute or chronic transplant rejection in a subject in need of such treatment, which method comprises administering to said subject an effective amount of a compound of formula (I) or a pharmaceutically acceptable salt thereof;
1.2 A method for preventing or treating autoimmune diseases, such as rheumatoid arthritis, systemic lupus erythematosus, inflammatory bowel disease, psoriasis, or multiple sclerosis in a subject in need of such treatment, which method comprises administering to said subject an effective amount of a compound of formula (I) or a pharmaceutically acceptable salt thereof;
1.3 A method for preventing or treating multiple sclerosis in a subject in need of such treatment, which method comprises administering to said subject an effective amount of a compound of formula (I) or a pharmaceutically acceptable salt thereof;
2. A compound of formula (I), in free form or in salt form, in particular in a pharmaceutically acceptable salt form for use as a pharmaceutical, e.g. in any of the methods as indicated under 1.1, 1.2 or 1.3 above.
3. A pharmaceutical composition, e.g. for use in any of the methods as in 1.1, 1.2 or 1.3 above comprising a compound of formula (I) in free form or pharmaceutically acceptable salt form in association with a pharmaceutically acceptable diluent or carrier therefor.
4. A compound of formula (I) or a salt thereof, in particular a pharmaceutically acceptable salt thereof for use in the preparation of a pharmaceutical composition for use in any of the method as in 1.1, 1.2 or 1.3 above.
5. A method as defined above comprising co-administration, e.g. concomitantly or in sequence, of a therapeutically effective non-toxic amount of a compound of formula (I) and at least a second drug substance, e.g. an immunosuppressant, immunomodulatory, anti-inflammatory or chemotherapeutic drug, e.g. as indicated below.
6. A pharmaceutical combination, e.g. a kit, comprising a) a first agent which is a compound of formula (I) as disclosed herein, in free form or in pharmaceutically acceptable salt form, and b) at least one co-agent, e.g. an immunosuppressant, immunomodulatory, anti-inflammatory, chemotherapeutic or anti-infectious agent. The kit may comprise instructions for its administration.

The present invention also pertains to each individual compound disclosed herein above, including novel intermediates. It further pertains to a method and/or a use as disclosed in the above sections 1.1, 1.2 and/or 1.3 in which method and/or use each individual compound, including any novel intermediate, may be used.

For the above uses the required dosage will of course vary depending on the mode of administration, the particular condition to be treated and the effect desired.

In general, satisfactory results are indicated to be obtained systemically at daily dosages of from about 0.03 to 5.0 mg/kg per body weight. An indicated daily dosage in the larger mammal, e.g. humans, is in the range from about 0.5 mg to about 500 mg, conveniently administered, for example, in divided doses up to four times a day or in retard form. Suitable unit dosage forms for oral administration comprise from ca. 0.1 to 50 mg active ingredient.

The compounds of formula (I) may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula (I) in free form or in pharmaceutically acceptable salt form in association with at least one pharmaceutical acceptable carrier or diluent may be manufactured in conventional manner by mixing with a pharmaceutically acceptable carrier or diluent.

The compounds of formula (I) may be administered in free form or in pharmaceutically acceptable salt form e.g. as indicated above. Such salts may be prepared in conventional manner and exhibit the same order of activity as the free compounds. A preferred route of administration for these compounds is parenterally, using a salt, for example a N-methyl-D-glucamine salt or D-glucamine salt.

The compounds of formula (I) in free form or in pharmaceutically acceptable salt form, exhibit still further valuable pharmacological properties such as for example an improved pharmacokinetic profile as being typically assessable by an ADME-study (ADME=absorption, distribution, metabolism and elimination).

The compounds of formula (I) may be administered as the sole active ingredient or in conjunction with, e.g. as an adjuvant to, other drugs e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g a malignant cell anti-proliferative agent. For example, the compounds of formula (I) may be used in combination with a calcineurin inhibitor, e.g. cyclosporin A or FK 506; a mTOR inhibitor, e.g. rapamycin, 40-O-(2-hydroxyethyl)-rapamycin, CCI779, ABT578, AP23573, AP23464, AP23675, AP23841, TAFA-93, biolimus-7 or biolimus-9; an ascomycin having immunosuppressive properties, e.g. ABT-281, ASM981, etc.; corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; sphingosine-1-phosphate receptor modulators such as FTY720 (fingolimod), or compounds disclosed in WO 2005/000833; immunosuppressive monoclonal antibodies, e.g., monoclonal antibodies to leukocyte receptors, e.g., MHC, CD2, CD3, CD4, CD7, CD8, CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; or an anti-infectious agent.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula (I) and a co-agent, are both administered to a patient simultaneously in the form of a single entity or dosage. The term “non-fixed combination” means that the active ingredients, e.g. a compound of formula (I) and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

1. A compound of formula (I) or a salt thereof; 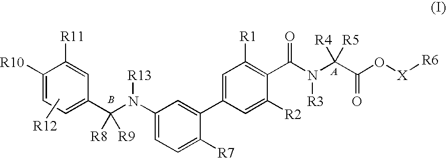
wherein
R1 is C₁-C₆ alkyl, halo, halo C₁-C₆ alkyl;
R2 is H, C₁-C₆ alkyl or halo;
R3 is H, or C₁-C₆ alkyl;
R4 is C₁-C₆ alkyl optionally substituted by halogen, hydroxyl, C₁-C₆ alkoxy or NR′R″, wherein
R′ and R″ are each independently selected from H, acyl and C₁-C₆ alkyl;
X is a bond or is C₁-C₆ alkylene optionally interrupted by 1-2 O-atoms;
R5 is H or C₁-C₆ alkyl; or
R4 and R5 together with the carbon atom to which they are attached form a 3-6 membered carbocyclic ring which is optionally interrupted by NR15;
R6 is H; C₁-C₆ alkyl optionally interrupted by 1-2 O-atoms; or C₁-C₆ alkyl substituted by NR16R17;
R7 is H or halo;
R8 is C₁-C₆ alkyl, optionally substituted by halo;
R9 is H, or C₁-C₆ alkyl, optionally substituted by halo;
R10 is C₁-C₆ alkoxy, OH, halo, cyano, or C₁-C₆ alkyl optionally substituted by halo;
R11 is C₁-C₆ alkoxy, OH, halo, cyano, or C₁-C₆ alkyl optionally substituted by halo;
R12 is H, C₁-C₆ alkoxy, OH, halo, cyano, or C₁-C₆ alkyl optionally substituted by halo;
R13 is H or C₁-C₆ alkyl; and
R15, R16 and R17 are independently selected from H, acyl and C₁-C₆ alkyl.

2. A compound of according to claim 1, which is a compound of formula (Ib) or a salt thereof, 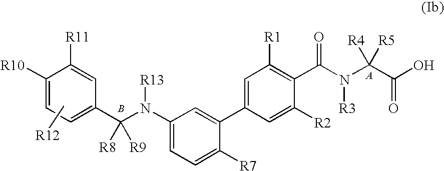
wherein
R1 is C₁-C₆ alkyl;
R2 is H or C₁-C₆ alkyl;
R3 is H;
R4 is C₁-C₆ alkyl optionally substituted by hydroxy,
R5 is H or C₁-C₆ alkyl; or
R4 and R5 together with the carbon atom to which they are attached form a 3-6 membered carbocyclic ring;
R7 is H or halo;
R8 is C₁-C₆ alkyl, optionally substituted by halo;
R9 is H, or C₁-C₆ alkyl, optionally substituted by halo;
R10 is halo, or C₁-C₆ alkyl optionally substituted by halo;
R11 is C₁-C₆ alkyl optionally substituted by halo;
R12 is H, C₁-C₆ alkoxy, OH, halo, cyano, or C₁-C₆ alkyl optionally substituted by halo; and
R13 is H.

3. A compound of claim 1 or 2, wherein R1 and R2 are both methyl.

4. A compound of claim 1 or 2, wherein R11 is methyl.

5. A compound of claim 1 or 2, wherein R10 is halo, in particular chloro.

6. A compound in accordance to any one of the preceding claims, wherein R8 is selected from halo C₁-C₆ alkyl, and C₁-C₆ alkyl and wherein R9 is H.

7. A compound in accordance to any one of the preceding claims, wherein R4 is C₁-C₆ alkyl and R5 is hydrogen, or wherein R4 and R5 together with the carbon atom to which they are attached form a 3-5 membered carbocyclic ring.

8. A compound in accordance to claims 1, 3-7, wherein X is a bond and R6 is H.

9. A compound in accordance to any one of the preceding claims, wherein R13 is hydrogen.

10. A process for the manufacture of a compound in accordance to any one of the preceding claims, comprising:
a) For compounds of formula (I) wherein R9 and R13 are H, the step of reductive amination between an aniline of formula (II) and a ketone of formula (III) using standard reducing agents, e.g. decaborane, sodium cyanoborohydride or sodium triacetoxyborohydride, followed by an optional deprotection step: 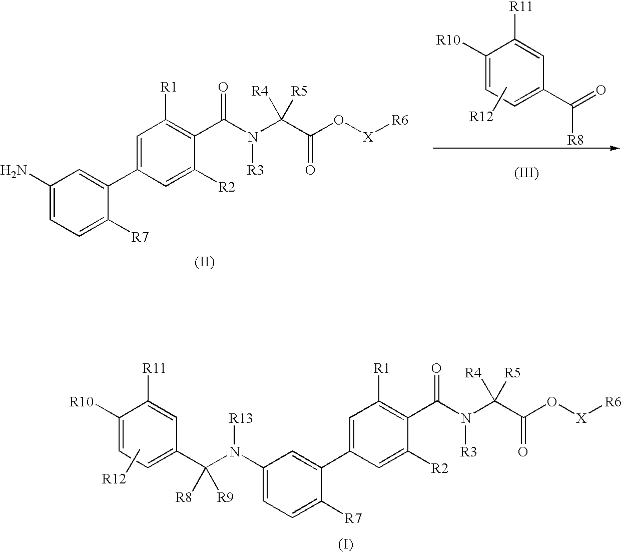
b) For compounds of formula (I) wherein R9 is H, the step of in situ double reductive amination between an aniline of formula (II) and a ketone of formula (III) followed by an aldehyde of formula (IV), wherein R′″ is H or C₁-C₅ alkyl, using standard reducing agents, e.g. decaborane, sodium cyanoborohydride or sodium triacetoxyborohydride, followed by an optional deprotection step: 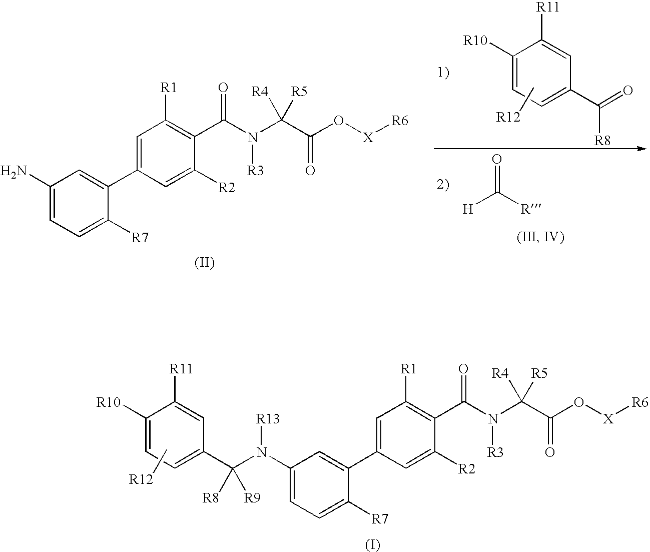
c) For compounds of formula (I) the step of coupling a carboxylic acid of formula (V) with an optionally protected amine of formula (VI) or a salt thereof using standard coupling reagents, e.g. TBTU or HATU, and a base, e.g. Hünig's base or triethyl amine, followed by an optional deprotection step: 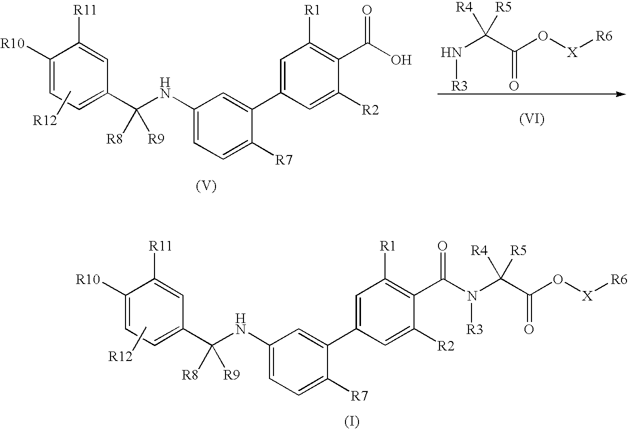
d) For compounds of formula (I) the step of palladium-catalyzed Suzuki coupling of a boronic acid derivative of formula (VIII) with a halide of formula (VII) or a salt thereof using standard palladium catalysts, e.g. Pd(PPh₃)₄ or PdCl₂(PPh₃)₂ or Pd(OAc)₂ with 2-dicyclohexylphosphino-2′,6′-dimethoxybiphenyl, and a base, e.g. sodium bicarbonate or potassium phosphate, followed by an optional deprotection step: 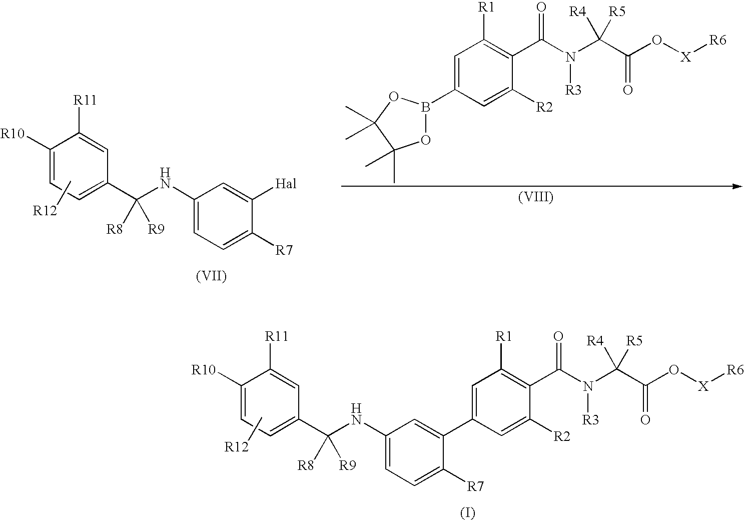
wherein the variables in the above disclosed formulae are as defined in the main claim.

11. A method for preventing or treating diseases or disorders which are mediated by lymphocytes interactions, in a subject in need of such treatment or prevention, which method comprises administering to said subject an effective amount of a compound of formula (I) or a pharmaceutically acceptable salt thereof.

12. Use of a compound of formula (I) according to claim 1 in the preparation of a medicament for the treatment and/or prevention of diseases or disorders mediated by lymphocytes interactions.

13. A compound of formula (I) according to claim 1 for the treatment and/or prevention of diseases or disorders mediated by lymphocytes interactions

14. The method, use or the compound of the preceding claims, wherein said diseases or disorders mediated by lymphocytes interactions pertain to transplantation, such as acute or chronic rejection of cell, tissue or organ allo- or xenografts or delayed graft function, graft versus host disease, autoimmune diseases, e.g. rheumatoid arthritis, systemic lupus erythematosus, hashimoto's thyroidis, multiple sclerosis, myasthenia gravis, neuropathic pain, Behcet's disease, Wegener's granulamatosis, ankylosing spondylitis, polymyositis, CIDP (Chronic Idiopathic Demyelinating Polyneuropathy), diabetes type I or II and the disorders associated therewith, vasculitis, pernicious anemia, Sjoegren syndrome, uveitis, psoriasis, Graves opthalmopathy, alopecia greata and others, allergic diseases, e.g. allergic asthma, atopic dermatitis, allergic rhinitis/conjunctivitis, allergic contact dermatitis, inflammatory diseases optionally with underlying aberrant reactions, e.g. inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, ischemia/reperfusion injury, e.g. myocardial infarction, stroke, gut ischemia, renal failure or hemorrhage shock, traumatic shock, cancer, e.g. breast cancer, T cell lymphomas or T cell leukemias, infectious diseases, e.g. toxic shock (e.g. superantigen induced), septic shock, adult respiratory distress syndrome or viral infections, e.g. AIDS, viral hepatitis, e.g. hepatitis B or C, chronic bacterial infection, or neurodegenerative diseases, e.g. Alzheimer disease, amyotrophic lateral sclerosis, or senile dementia, wherein examples of cell, tissue or solid organ transplants include e.g. pancreatic islets, stem cells, bone marrow, corneal tissue, neuronal tissue, heart, lung, combined heart-lung, kidney, liver, bowel, pancreas, trachea or oesophagus, deregulated angiogenesis e.g. diseases caused by ocular neovascularisation, especially retinopathies (diabetic retinopathy, age-related macular degeneration); psoriasis; haemangioblastomas, such as “strawberry-marks” (=haemangioma); various inflammatory diseases, such as arthritis, especially rheumatoid arthritis, arterial atherosclerosis and atherosclerosis occurring after transplants, endometriosis or chronic asthma; and, especially, tumor diseases (solid tumors, but also leukemias and other liquid tumors).

15. The method, use or the compound of the preceding claims, wherein said disease or said disorder is selected from an autoimmune disease, such as rheumatoid arthritis, systemic lupus erythematosus, inflammatory bowel disease, psoriasis, or multiple sclerosis.

16. A combination, e.g. a pharmaceutical combination or a kit, comprising a) a first agent which is a compound of formula (I) as disclosed in claim 1, in free form or in salt form, in particular in pharmaceutically acceptable salt form, and b) at least one co-agent, e.g. an immunosuppressant, immunomodulatory, anti-inflammatory, chemotherapeutic or anti-infectious agent.

17. A pharmaceutical composition, in particular for use in any of the methods of the preceding claims, comprising a compound of formula (I) of claim 1 in free form or pharmaceutically acceptable salt form in association with a pharmaceutically acceptable diluent or carrier therefore.

18. A method as provided in the preceding claims, e.g. claim 10, 14 or 15, comprising co-administration, e.g. concomitantly or in sequence, of a therapeutically effective amount of a compound of formula (I) in accordance to claim 1 and at least a second drug substance, e.g. an immunosuppressant, immunomodulatory, anti-inflammatory or chemotherapeutic drug, e.g. as disclosed in the description.
